SEQUENCE LISTING (142 sequences)

SEQ ID NO: 1 - DNA, 20 bp, synthetic construct
  source 1..20: mol_type = other DNA
ggtaacccgttgaaccccat

SEQ ID NO: 2 - DNA, 20 bp, synthetic construct
  source 1..20: mol_type = other DNA
caacgcaagcttatgacccg

SEQ ID NO: 3 - DNA, 21 bp, synthetic construct
  source 1..21: mol_type = other DNA
aaaaccgcagacatccttcag

SEQ ID NO: 4 - DNA, 21 bp, synthetic construct
  source 1..21: mol_type = other DNA
cataccgaaactcgttcaccc

SEQ ID NO: 5 - DNA, 19 bp, synthetic construct
  source 1..19: mol_type = other DNA
cccgtcatgcagtcgcttt

SEQ ID NO: 6 - DNA, 21 bp, synthetic construct
  source 1..21: mol_type = other DNA
cactgggtcgaacaaattgcc

SEQ ID NO: 7 - DNA, 21 bp, synthetic construct
  source 1..21: mol_type = other DNA
gatgtgggagtttggagacag

SEQ ID NO: 8 - DNA, 21 bp, synthetic construct
  source 1..21: mol_type = other DNA
gctgaatttgtccatagggct

SEQ ID NO: 9 - DNA, 22 bp, synthetic construct
  source 1..22: mol_type = other DNA
ctttcctactctgtacccgagg

SEQ ID NO: 10 - DNA, 21 bp, synthetic construct
  source 1..21: mol_type = other DNA
cggggcattccattgataagg

SEQ ID NO: 11 - DNA, 21 bp, synthetic construct
  source 1..21: mol_type = other DNA
taacttcctcactcgaagcca

SEQ ID NO: 12 - DNA, 22 bp, synthetic construct
  source 1..22: mol_type = other DNA
agttccatgacccatctctgtc

SEQ ID NO: 13 - DNA, 20 bp, synthetic construct
  source 1..20: mol_type = other DNA
atggcgctcttcacgaaatc

SEQ ID NO: 14 - DNA, 21 bp, synthetic construct
  source 1..21: mol_type = other DNA
actggtcgaaggggtcatcaa

SEQ ID NO: 15 - DNA, 22 bp, synthetic construct
  source 1..22: mol_type = other DNA
acttatcgctaactccctgact

SEQ ID NO: 16 - DNA, 19 bp, synthetic construct
  source 1..19: mol_type = other DNA
acaccagcatctccgacga

SEQ ID NO: 17 - DNA, 21 bp, synthetic construct
  source 1..21: mol_type = other DNA
agttgctctacctcgggatct

SEQ ID NO: 18 - DNA, 21 bp, synthetic construct
  source 1..21: mol_type = other DNA
atgccacatcaaagcgaaagc

SEQ ID NO: 19 - DNA, 22 bp, synthetic construct
  source 1..22: mol_type = other DNA
gctacttggagggattagtgcg

SEQ ID NO: 20 - DNA, 23 bp, synthetic construct
  source 1..23: mol_type = other DNA
gcggaactctactaccatcttct

SEQ ID NO: 21 - DNA, 21 bp, synthetic construct
  source 1..21: mol_type = other DNA
ctacccaaaatccgcgatgag

SEQ ID NO: 22 - DNA, 21 bp, synthetic construct
  source 1..21: mol_type = other DNA
ccatgtcaccttccaatcgaa

SEQ ID NO: 23 - DNA, 19 bp, synthetic construct
  source 1..19: mol_type = other DNA
atgcggggaatcgtgaacg

SEQ ID NO: 24 - DNA, 21 bp, synthetic construct
  source 1..21: mol_type = other DNA
aggctgggataggtagttccg

SEQ ID NO: 25 - DNA, 22 bp, synthetic construct
  source 1..22: mol_type = other DNA
ggcaaagaccggattgaaatct

SEQ ID NO: 26 - DNA, 23 bp, synthetic construct
  source 1..23: mol_type = other DNA
gtcgaaatcgaagagttaaggca

SEQ ID NO: 27 - DNA, 19 bp, synthetic construct
  source 1..19: mol_type = other DNA
ctggtggcgggtgctttag

SEQ ID NO: 28 - DNA, 21 bp, synthetic construct
  source 1..21: mol_type = other DNA
gctacgtctgggattcgatct

SEQ ID NO: 29 - DNA, 21 bp, synthetic construct
  source 1..21: mol_type = other DNA
ggatgctgctgtcccaactaa

SEQ ID NO: 30 - DNA, 21 bp, synthetic construct
  source 1..21: mol_type = other DNA
tctcttgcttgtcctcggaac

SEQ ID NO: 31 - DNA, 22 bp, synthetic construct
  source 1..22: mol_type = other DNA
ctcccaagtcacacaagaactg

SEQ ID NO: 32 - DNA, 23 bp, synthetic construct
  source 1..23: mol_type = other DNA
ccagctcctttttgtaagccttt

SEQ ID NO: 33 - DNA, 20 bp, synthetic construct
  source 1..20: mol_type = other DNA
gtccagaatgggaagtggga

SEQ ID NO: 34 - DNA, 20 bp, synthetic construct
  source 1..20: mol_type = other DNA
cactgcatgtctctttggcg

SEQ ID NO: 35 - DNA, 21 bp, synthetic construct
  source 1..21: mol_type = other DNA
ttctggtgcttgtctcactga

SEQ ID NO: 36 - DNA, 22 bp, synthetic construct
  source 1..22: mol_type = other DNA
cagtatgttcggcttcccattc

SEQ ID NO: 37 - DNA, 20 bp, synthetic construct
  source 1..20: mol_type = other DNA
caggacgtgagagtcgatgg

SEQ ID NO: 38 - DNA, 20 bp, synthetic construct
  source 1..20: mol_type = other DNA
ctctgcctatgctgccttca

SEQ ID NO: 39 - DNA, 21 bp, synthetic construct
  source 1..21: mol_type = other DNA
tggtgtcccacatctacatca

SEQ ID NO: 40 - DNA, 22 bp, synthetic construct
  source 1..22: mol_type = other DNA
cagcgtccatattctgtttcca

SEQ ID NO: 41 - DNA, 21 bp, synthetic construct
  source 1..21: mol_type = other DNA
ctggaagcctggtatgaggat

SEQ ID NO: 42 - DNA, 22 bp, synthetic construct
  source 1..22: mol_type = other DNA
cagggtcaagagtagtgaaggt

SEQ ID NO: 43 - DNA, 23 bp, synthetic construct
  source 1..23: mol_type = other DNA
ggatgtgcattctatggtgtacc

SEQ ID NO: 44 - DNA, 23 bp, synthetic construct
  source 1..23: mol_type = other DNA
tttcgggattgcttatctcagac

SEQ ID NO: 45 - DNA, 22 bp, synthetic construct
  source 1..22: mol_type = other DNA
gctggtcttcggatgaaaatga

SEQ ID NO: 46 - DNA, 23 bp, synthetic construct
  source 1..23: mol_type = other DNA
ccacaaaggtgatattggaaccc

SEQ ID NO: 47 - DNA, 22 bp, synthetic construct
  source 1..22: mol_type = other DNA
ggacacatggcctagaataacg

SEQ ID NO: 48 - DNA, 21 bp, synthetic construct
  source 1..21: mol_type = other DNA
agacacacccttagagaacgg

SEQ ID NO: 49 - DNA, 21 bp, synthetic construct
  source 1..21: mol_type = other DNA
acttggggaccacctattcct

SEQ ID NO: 50 - DNA, 19 bp, synthetic construct
  source 1..19: mol_type = other DNA
atcgccaatcagacgctcc

SEQ ID NO: 51 - DNA, 22 bp, synthetic construct
  source 1..22: mol_type = other DNA
gatttacgcacgatgacacagt

SEQ ID NO: 52 - DNA, 22 bp, synthetic construct
  source 1..22: mol_type = other DNA
acctgcaaagacattagacagc

SEQ ID NO: 53 - DNA, 21 bp, synthetic construct
  source 1..21: mol_type = other DNA
tactcagcagaccttgaacct

SEQ ID NO: 54 - DNA, 21 bp, synthetic construct
  source 1..21: mol_type = other DNA
cagtcttggcagcaagttgac

SEQ ID NO: 55 - DNA, 21 bp, synthetic construct
  source 1..21: mol_type = other DNA
ggagagggaatgatgttgcct

SEQ ID NO: 56 - DNA, 21 bp, synthetic construct
  source 1..21: mol_type = other DNA
tcctgcttcgtgttacacaca

SEQ ID NO: 57 - DNA, 22 bp, synthetic construct
  source 1..22: mol_type = other DNA
ctggtgtgtgacgttcccatta

SEQ ID NO: 58 - DNA, 18 bp, synthetic construct
  source 1..18: mol_type = other DNA
ccgacagcacgaggcttt

SEQ ID NO: 59 - DNA, 20 bp, synthetic construct
  source 1..20: mol_type = other DNA
ggccttccctacttcacaag

SEQ ID NO: 60 - DNA, 20 bp, synthetic construct
  source 1..20: mol_type = other DNA
atttccacgatttcccagag

SEQ ID NO: 61 - DNA, 21 bp, synthetic construct
  source 1..21: mol_type = other DNA
tcttttcctcggagcatgaca

SEQ ID NO: 62 - DNA, 23 bp, synthetic construct
  source 1..23: mol_type = other DNA
gacctctctactcacttctccag

SEQ ID NO: 63 - DNA, 20 bp, synthetic construct
  source 1..20: mol_type = other DNA
tctggaccctgcattctgag

SEQ ID NO: 64 - DNA, 23 bp, synthetic construct
  source 1..23: mol_type = other DNA
cactagggaatccccagtaagag

SEQ ID NO: 65 - DNA, 21 bp, synthetic construct
  source 1..21: mol_type = other DNA
tgttcgttttcaccattggga

SEQ ID NO: 66 - DNA, 21 bp, synthetic construct
  source 1..21: mol_type = other DNA
cgcatgattatctcggtcaca

SEQ ID NO: 67 - DNA, 21 bp, synthetic construct
  source 1..21: mol_type = other DNA
gggagtttggctccagagttt

SEQ ID NO: 68 - DNA, 21 bp, synthetic construct
  source 1..21: mol_type = other DNA
tgtgtcttcaggggtccttag

SEQ ID NO: 69 - DNA, 21 bp, synthetic construct
  source 1..21: mol_type = other DNA
ctgattctgcaacggagttgt

SEQ ID NO: 70 - DNA, 19 bp, synthetic construct
  source 1..19: mol_type = other DNA
gacgaagctctgtcggctc

SEQ ID NO: 71 - DNA, 21 bp, synthetic construct
  source 1..21: mol_type = other DNA
atgtcttcccccacaagttct

SEQ ID NO: 72 - DNA, 20 bp, synthetic construct
  source 1..20: mol_type = other DNA
gaccacgatgtaggcagagc

SEQ ID NO: 73 - DNA, 21 bp, synthetic construct
  source 1..21: mol_type = other DNA
aggcgaactccacagaatgtt

SEQ ID NO: 74 - DNA, 22 bp, synthetic construct
  source 1..22: mol_type = other DNA
acaaaagaggtactgtccgtct

SEQ ID NO: 75 - DNA, 21 bp, synthetic construct
  source 1..21: mol_type = other DNA
ccaagtgtggctcattaggca

SEQ ID NO: 76 - DNA, 23 bp, synthetic construct
  source 1..23: mol_type = other DNA
ccaatcttcggactggactctgt

SEQ ID NO: 77 - DNA, 23 bp, synthetic construct
  source 1..23: mol_type = other DNA
agggtttagttttgagttgacgg

SEQ ID NO: 78 - DNA, 21 bp, synthetic construct
  source 1..21: mol_type = other DNA
ccccgcttttgtcatattccg

SEQ ID NO: 79 - DNA, 18 bp, synthetic construct
  source 1..18: mol_type = other DNA
ccagcctactcattggga

SEQ ID NO: 80 - DNA, 19 bp, synthetic construct
  source 1..19: mol_type = other DNA
gggcctgctgttcacagtt

SEQ ID NO: 81 - DNA, 19 bp, synthetic construct
  source 1..19: mol_type = other DNA
gcgtcggagacagtgtttg

SEQ ID NO: 82 - DNA, 23 bp, synthetic construct
  source 1..23: mol_type = other DNA
cgtccatctaaagtgctgatgat

SEQ ID NO: 83 - DNA, 21 bp, synthetic construct
  source 1..21: mol_type = other DNA
ctgtgtgcaatgcctatgaga

SEQ ID NO: 84 - DNA, 21 bp, synthetic construct
  source 1..21: mol_type = other DNA
ctggagggtattgaagagccg

SEQ ID NO: 85 - DNA, 21 bp, synthetic construct
  source 1..21: mol_type = other DNA
gaccttgtgtcctccgcttat

SEQ ID NO: 86 - DNA, 19 bp, synthetic construct
  source 1..19: mol_type = other DNA
caaccgcaatttgtggctc

SEQ ID NO: 87 - DNA, 23 bp, synthetic construct
  source 1..23: mol_type = other DNA
atgtctagcaatggtacagatgc

SEQ ID NO: 88 - DNA, 22 bp, synthetic construct
  source 1..22: mol_type = other DNA
cgtggaactgataagaggcagg

SEQ ID NO: 89 - DNA, 21 bp, synthetic construct
  source 1..21: mol_type = other DNA
gtgtccaccaactcacagatg

SEQ ID NO: 90 - DNA, 21 bp, synthetic construct
  source 1..21: mol_type = other DNA
ggaccattccttttgcagcat

SEQ ID NO: 91 - DNA, 21 bp, synthetic construct
  source 1..21: mol_type = other DNA
tgtccagtgcttacaactcgg

SEQ ID NO: 92 - DNA, 20 bp, synthetic construct
  source 1..20: mol_type = other DNA
cagggcacaggtagtcacac

SEQ ID NO: 93 - DNA, 21 bp, synthetic construct
  source 1..21: mol_type = other DNA
tccgtggctgtctactaaaga

SEQ ID NO: 94 - DNA, 23 bp, synthetic construct
  source 1..23: mol_type = other DNA
tgggatatgatgacgttctctcc

SEQ ID NO: 95 - DNA, 20 bp, synthetic construct
  source 1..20: mol_type = other DNA
agagccccatctgtcctctc

SEQ ID NO: 96 - DNA, 22 bp, synthetic construct
  source 1..22: mol_type = other DNA
actggtagtctgcaaaaccaaa

SEQ ID NO: 97 - DNA, 21 bp, synthetic construct
  source 1..21: mol_type = other DNA
ggaagaccactcgcattcctt

SEQ ID NO: 98 - DNA, 22 bp, synthetic construct
  source 1..22: mol_type = other DNA
gtaatcagcaaccattgggtca

SEQ ID NO: 99 - DNA, 20 bp, synthetic construct
  source 1..20: mol_type = other DNA
ccctgtgcgtcagagactac

SEQ ID NO: 100 - DNA, 20 bp, synthetic construct
  source 1..20: mol_type = other DNA
caagctgttcgtgatgagcc

SEQ ID NO: 101 - DNA, 21 bp, synthetic construct
  source 1..21: mol_type = other DNA
ttcgacgatgacgtggaagag

SEQ ID NO: 102 - DNA, 21 bp, synthetic construct
  source 1..21: mol_type = other DNA
ggtactgctgcctgtcaatgg

SEQ ID NO: 103 - DNA, 19 bp, synthetic construct
  source 1..19: mol_type = other DNA
tgacccggctattccgtga

SEQ ID NO: 104 - DNA, 21 bp, synthetic construct
  source 1..21: mol_type = other DNA
ctgggctgagcaatacagttc

SEQ ID NO: 105 - DNA, 19 bp, synthetic construct
  source 1..19: mol_type = other DNA
gcagcaacgggaccattct

SEQ ID NO: 106 - DNA, 23 bp, synthetic construct
  source 1..23: mol_type = other DNA
ccccatgactaagtccttcaact

SEQ ID NO: 107 - DNA, 22 bp, synthetic construct
  source 1..22: mol_type = other DNA
aagtatcagcggtgcagaattt

SEQ ID NO: 108 - DNA, 23 bp, synthetic construct
  source 1..23: mol_type = other DNA
ccttgacaggatctatcattcgc

SEQ ID NO: 109 - DNA, 19 bp, synthetic construct
  source 1..19: mol_type = other DNA
ctcccgtggcttctagtgc

SEQ ID NO: 110 - DNA, 22 bp, synthetic construct
  source 1..22: mol_type = other DNA
gccttagtttggacaggatctg

SEQ ID NO: 111 - DNA, 22 bp, synthetic construct
  source 1..22: mol_type = other DNA
gcctttcagggaattaagctcc

SEQ ID NO: 112 - DNA, 22 bp, synthetic construct
  source 1..22: mol_type = other DNA
agatcaaccgatggacgtgtaa

SEQ ID NO: 113 - DNA, 20 bp, synthetic construct
  source 1..20: mol_type = other DNA
tcttccggcagtacgtgatg

SEQ ID NO: 114 - DNA, 21 bp, synthetic construct
  source 1..21: mol_type = other DNA
cggcgcagactatgaacatga

SEQ ID NO: 115 - DNA, 25 bp, synthetic construct
  source 1..25: mol_type = other DNA
gaccctcacactcagatcatcttct

SEQ ID NO: 116 - DNA, 20 bp, synthetic construct
  source 1..20: mol_type = other DNA
cctccacttggtggtttgct

SEQ ID NO: 117 - DNA, 19 bp, synthetic construct
  source 1..19: mol_type = other DNA
gggagtcctctcggaaagg

SEQ ID NO: 118 - DNA, 22 bp, synthetic construct
  source 1..22: mol_type = other DNA
ccggatagctggtgtacttgta

SEQ ID NO: 119 - DNA, 22 bp, synthetic construct
  source 1..22: mol_type = other DNA
ctccaggaatcaagagacctcc

SEQ ID NO: 120 - DNA, 19 bp, synthetic construct
  source 1..19: mol_type = other DNA
ccgggtccagtgaggatct

SEQ ID NO: 121 - DNA, 21 bp, synthetic construct
  source 1..21: mol_type = other DNA
ctgacccccaatcctgggtat

SEQ ID NO: 122 - DNA, 19 bp, synthetic construct
  source 1..19: mol_type = other DNA
ggcccggaacttgtcacat

SEQ ID NO: 123 - DNA, 19 bp, synthetic construct
  source 1..19: mol_type = other DNA
gggagtgccggaaatgacc

SEQ ID NO: 124 - DNA, 21 bp, synthetic construct
  source 1..21: mol_type = other DNA
tgaagtgttcagacgtggctt

SEQ ID NO: 125 - DNA, 21 bp, synthetic construct
  source 1..21: mol_type = other DNA
agtcaactgattgtggaacgc

SEQ ID NO: 126 - DNA, 21 bp, synthetic construct
  source 1..21: mol_type = other DNA
tctggaagggtattcgctgtc

SEQ ID NO: 127 - DNA, 25 bp, synthetic construct
  source 1..25: mol_type = other DNA
tctgacagagactgttgctcaactt

SEQ ID NO: 128 - DNA, 22 bp, synthetic construct
  source 1..22: mol_type = other DNA
agctggggcattattggatgaa

SEQ ID NO: 129 - DNA, 20 bp, synthetic construct
  source 1..20: mol_type = other DNA
aagcgctttaaatggagccc

SEQ ID NO: 130 - DNA, 20 bp, synthetic construct
  source 1..20: mol_type = other DNA
cttctgcgcctgacagagag

SEQ ID NO: 131 - DNA, 20 bp, synthetic construct
  source 1..20: mol_type = other DNA
tatttgtgcccggtaggcag

SEQ ID NO: 132 - DNA, 20 bp, synthetic construct
  source 1..20: mol_type = other DNA
ttcacgcttgtgttccacct

SEQ ID NO: 133 - DNA, 20 bp, synthetic construct
  source 1..20: mol_type = other DNA
agtgttagggcacacggaag

SEQ ID NO: 134 - DNA, 20 bp, synthetic construct
  source 1..20: mol_type = other DNA
cccacgcaaacaataccacc

SEQ ID NO: 135 - DNA, 20 bp, synthetic construct
  source 1..20: mol_type = other DNA
tgagggtgacctaacaacgc

SEQ ID NO: 136 - DNA, 20 bp, synthetic construct
  source 1..20: mol_type = other DNA
cctccctgaacagaggttcg

SEQ ID NO: 137 - DNA, 22 bp, synthetic construct
  source 1..22: mol_type = other DNA
tcaatgccaccttaatgctgtt

SEQ ID NO: 138 - DNA, 19 bp, synthetic construct
  source 1..19: mol_type = other DNA
cctggaatgcctacctggc

SEQ ID NO: 139 - DNA, 20 bp, synthetic construct
  source 1..20: mol_type = other DNA
tcatggcaggccatagttct

SEQ ID NO: 140 - DNA, 20 bp, synthetic construct
  source 1..20: mol_type = other DNA
gcgggctccaagagtacatt

SEQ ID NO: 141 - DNA, 20 bp, synthetic construct
  source 1..20: mol_type = other DNA
aagcgctttaaatggagccc

SEQ ID NO: 142 - DNA, 20 bp, synthetic construct
  source 1..20: mol_type = other DNA
cttctgcgcctgacagagag

(19) United States
(12) Patent Application Publication
(10) Pub. No.: US 2026/0256960 A1
(43) Pub. Date: Sep. 3, 2026

(54) TARGETING LYSOSOMAL LIPID IN ALZHEIMER'S DISEASE

(71) Applicant: The University of North Carolina at Chapel Hill, Chapel Hill, NC (US)

(72) Inventors: Leon G. Coleman, JR., Chapel Hill, NC (US); Alexandra Barnett, Charlotte, NC (US); Zibo Li, Chapel Hill, NC (US); Saradhi Kalari, Chapel Hill, NC (US); Xinrui Ma, Carrboro, NC (US)

(21) Appl. No.: 19/164,778

(22) PCT Filed: Mar. 22, 2024
(86) PCT No.: PCT/US2024/021194

§ 371 Date: Sep. 12, 2025

Related U.S. Application Data
(60) Provisional application No. 63/491,573, filed on Mar. 22, 2023.

(51) Int. Cl. A61K 51/04; A61B 5/00; C07D 417/12

(52) U.S. Cl. CPC A61K 51/0497; A61B 5/4064; A61B 5/4088; A61K 51/0453; A61K 51/0455; C07D 417/12

(57) ABSTRACT

Novel lysosomal acid lipase (LAL) positron emission tomography ligands are provided. Also disclosed herein are methods of assessing the risk of developing Alzheimer's disease (AD) or Alzheimer's Disease Related Dementias (ADRD) and methods of diagnosing AD/ADRD in a subject comprising measuring levels of LAL and optionally LAL accumulation in the subject. Methods of treatment comprising administering LAL are also provided.

### STATEMENT OF PRIORITY

This application claims the benefit, under 35 U.S.C. § 119(e), of U.S. Provisional Application No. 63/491,573, filed Mar. 22, 2023, the entire contents of which are incorporated by reference herein in their entirety.

### GOVERNMENT SUPPORT

This invention was made with government support under Grant No. 1R01AA028924-01 awarded by National Institutes of Health (NIH). The government has certain rights in the invention.

### SEQUENCE LISTING

A Sequence Listing in XML text format, submitted under 37 C.F.R. § 1.821-1.834, entitled 5470-946WO_ST26.xml, 126,109 bytes in size, generated on Mar. 20, 2024 and filed electronically, is provided in lieu of a paper copy. This Sequence Listing is hereby incorporated herein by reference into the specification for its disclosures.

### FIELD OF THE INVENTION

This invention relates to methods of using lysosomal acid lipase in subjects having or at risk or developing Alzheimer's Disease and Alzheimer's Disease Related Dementias. In particular, compositions and methods to treat Alzheimer's Disease and Alzheimer's Disease Related Dementias are also provided.

### BACKGROUND OF THE INVENTION

Alzheimer's disease (AD) is the most common form of dementia with over 6 million Americans over the age of 65 living with AD. AD has a traditionally defined pathological progression—the accumulation of pathological amyloid (Aβ) and tau species that emerges with aging. Over 90% of AD cases are considered sporadic, and though there is often no clear etiology for the disease, several key modifiable lifestyle behaviors have been identified. These include, but are not limited to, heavy smoking or alcohol use, diabetes, hypertension, and obesity during midlife 1-5. Though these risk factors have diverse biological effects, they converge upon a common pathological progression. This suggests various environmental insults cause shared neuronal and/or glial dysfunction that becomes insurmountable with aging. Several rodent models reproduce Aβ and tau pathology, typically through expression of rare human familial AD transgenes. The 3×Tg-AD triple transgenic mouse model (APPSwe, tauP301, Psen1 tm1Mpm) features a progressive age-related, caudal-rostral, increase in pathologic Aβ and tau species (Oddo et al., 2003a; Oddo et al., 2003b). Similar to findings in human AD, pathology is seen first in entorhinal and hippocampal regions, followed later by cortex (Belfiore et al., 2019). Though the 3×Tg-AD model features continuous expression of mutated genes found in familial AD, it shares a requisite feature seen in sporadic AD—the emergence of pathology at later ages. This suggests that protective or maintenance cellular processes are progressively lost, resulting in the unmasking of AD pathology with aging. Therefore, modifiable risk factors associated with sporadic AD that accelerate disease progression can be used in this model to identify such underlying age-related vulnerabilities. This approach was used to uncover underlying cellular and molecular drivers of AD pathology that may be relevant to a large population of individuals with sporadic/late-onset AD.

Though the precise involvement of Aβ plaques to AD symptomatology is currently a subject of debate, intraneuronal Aβ₁₋₄₂ accumulation in humans and rodents is certainly related to neurotoxicity and cognitive deficits. Intraneuronal Aβ₁₋₄₂ is a cleaved form of Aβ that is found with early AD-pathology. In both wild-type (WT) and disease states, intraneuronal Aβ₁₋₄₂ is trafficked primarily to the endosomal/multivesicular body (MVB) compartment, originating either from internalization and β-secretase (BACE)-mediated cleavage of membrane amyloid precursor protein (APP) or endocytosis of extracellular Aβ₁₋₄₂ aggregates. Degradation of endosomal contents involves lysosomal incorporation either through fusion with autophagosomes or endolysosome formation. Autophagic and lysosomal dysfunction have been proposed in AD, with studies suggesting reduced acidification of lysosomes promotes intracellular Aβ₁₋₄₂ accumulation. However, to Applicant's knowledge, specific molecular and/or cell underpinnings of lysosomal and/or autophagic dysfunction relevant to broad populations of individuals with sporadic AD are unknown.

Human studies suggest a role for aberrant lipid metabolism in AD, though the cellular underpinnings have yet to be resolved. For instance, the ε4 polymorphism of APOE, a key regulator of lipid transport, uptake, and distribution across cell types, is one of the strongest genetic risk factors for AD. Further, polymorphisms in the lipid efflux proteins such as ABCA1, ABCA2, and ABCA7 are associated with increased risk for AD. In most parenchymal cells such as hepatocytes and adipocytes intracellular lipid metabolism involves coordinated actions of lipolysis and lysosomal digestion of lipid droplets. Lipophagy is a specialized form of autophagy that involves degradation of cytosolic neutral lipid droplets in the lysosome. The lysosomal acid lipase (LAL) facilitates lipid breakdown during lipophagy with subsequent release of fatty acids (FAs) into the cytosol. Neurons typically rely on glucose for energy production, thus the contribution of FAs produced de novo to neuronal activity is unclear, given greater oxygen requirement for and the toxic byproducts produced by β-oxidation. Nonetheless, efficient lipophagy is required to prevent the lysosomal accumulation of lipids which may cause lysosomal damage. For instance, in lysosomal storage disorders such as Niemann-Pick type C disease, lysosomal lipid accumulation causes lysosomal and autophagic dysfunction, ultimately resulting in neuronal death. To date, a role for neuronal lipophagy dysfunction has not been implicated in AD.

There is a need in the art for additional methods of assessing patients at risk of developing AD and Alzheimer's Disease Related Dementias (ADRD) and treatment of AD and ADRD.

### SUMMARY OF THE INVENTION

The present invention is based on the identification of lysosomal acid lipase (LAL) insufficiency and accumulation of lipid in neuronal lysosomes as causative in AD and ADRD pathogenesis. The invention further relates to LAL ligands useful in the methods of the invention.

Thus, in one aspect, a LAL positron emission tomography (PET) ligand is provided according to:

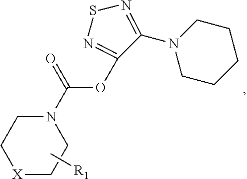

wherein X is C, CH, CH₂, CR₂, S, N, or O, wherein R₁ can be substituted at one or more positions on the piperidyl ring and each R₁ is independently a C₁-C₅ alkyl, C₁-C₅ cyano, C₁-C₅ amide group, a linker (e.g., a PEG linker or an arene-containing linker), or a halogen, and comprises a radioisotope, and wherein R₂ comprises a radiolabel. In an aspect, a composition comprising an LAL PET ligand of the invention is provided.

An aspect of the invention relates to a method of assessing the risk of developing AD or ADRD in a subject. The method can comprise the steps of: (a) measuring the amount and/or activity of lysosomal acid lipase (LAL) in the subject; (b) comparing the amount and/or activity of LAL measured in (a) with a threshold amount and/or activity of LAL; and (c) identifying an increased risk of developing AD or ADRD based on the amount and/or activity of LAL measured in (a) being lower than the threshold amount and/or activity of LAL. The method can further comprise a step of administering a therapy to treat the subject based on the diagnosis or identification of the risk of developing AD/ADRD.

Another aspect of the invention is a method of diagnosing AD or ADRD in a subject. The method can comprise the steps of: (a) measuring the amount and/or activity of LAL in the subject; (b) comparing the amount and/or activity of LAL measured in (a) with a threshold amount and/or activity of LAL; and (c) diagnosing AD or ADRD based on the amount and/or activity of LAL measured in (a) being lower than the threshold amount and/or activity of LAL. The method can further comprise a step of administering a therapy to treat the subject based on the diagnosis or identification of the risk of developing AD/ADRD.

Another aspect of the invention is a method of treating a subject diagnosed with or at risk of developing AD/ADRD, the method comprising: (a) performing the method described herein to determine whether the subject has AD/ADRD or has an increased probability of developing AD/ADRD; and (b) administering a therapy to the subject based on the results of (a).

An additional aspect of the invention is a method of treating AD/ADRD in a subject in need thereof, the method comprising administering LAL to the subject, thereby treating AD/ADRD.

In a further aspect, a method for slowing the progression of AD/ADRD in a subject in need thereof is provided, comprising administering LAL to the subject, thereby slowing the progression of AD/ADRD. LAL can be provided as an enzyme therapy, or as a polynucleotide encoding LAL as described herein.

In another aspect, methods of diagnosing AD/ADRD in a subject are provided, comprising the steps of (a) measuring the amount of LAL in the subject; (b) comparing the amount of LAL in the subject with the amount of LAL in a sample population of healthy subjects and subjects with AD/ADRD; (c) performing one or more standard AD/ADRD diagnostic tests on the subject; and (d) diagnosing whether the subject has AD/ADRD based on the results of (b) and (c).

A further aspect relates to methods for monitoring the response to LAL therapy in a subject, comprising measuring the amount and/or activity of LAL one or more times in a subject undergoing LAL therapy to determine changes in the amount and/or activity of LAL over time. In some embodiments, the subject is being treated for AD, ADRD, or LAL deficiency (LAL-D) disorder.

Another aspect relates to the use of LAL to treat AD/ADRD in a subject in need thereof.

An additional aspect relates to the use of LAL in the preparation of a medicament to treat AD/ADRD.

These and other aspects of the invention are set forth in more detail in the description of the invention below.

### BRIEF DESCRIPTION OF THE DRAWINGS

FIGS. 1A-1CC. Midlife alcohol and obesity increase LOAD pathology. (1A, 1B) Ethanol increased intraneuronal Aβ₁₋₄₂ in ENT 57%. N=15 control, 16 ethanol. (1C, 1D) Ethanol increased Aβ₁₋₄₂ in FCX in both sexes. *p<0.05, **p<0.01 Sidak's post-test. Scale: 100 μm (1E, 1F) Ethanol increased Aβ plaques in the female subiculum by 91% (dorsal) and 127% (ventral). Scale bar: 200 μm. Ethanol increased p-tau181 in (1G) CA1 and (1H) FCX. N=18 control, 19 ethanol. (I, J) 40% increase in p-tau181 in FCX. Scale bar: 50 μm. (1K, 1L) Ethanol increased TUNEL+neurodegeneration in ENT by 3-fold. N=6/group. (1M, 1N) 5 weeks of ethanol increased total Aβ in ENT of WT 10-mo mice. (1O, 1P) Obesity increased ENT Aβ₁₋₄₂ by 47%. N=7/group. (1Q, 1R) Obesity increased intraneuronal Aβ₁₋₄₂ in FCX 2-fold in males and 40% in females. (1S, 1T) 2.4-fold increase in Aβ plaques in the dorsal of obese females. Scale bar: 200 μm. **p<0.01, Sidak's multiple comparison test. (1U, 1V) 41% increase in p-tau181 in CA1 in obese mice. N=6 Control, 7 obese. p=0.05. (1W) Braak staging scores were slightly increased in AUD. *p<0.05, paired t-test. (1X, 1Y) Human AUD vm-PFC had a ~50% increase in p-tau214 and a ~30% increase in p-tau181 with no change in total tau. **p<0.01, paired t-test, N=16/group. (1Z, 1AA) Human AUD hippocampus showed a 27% increase in p-tau214 and a 20% increase in p-tau181 by Western blot. **p<0.01. (1BB, 1CC) Aβ₁₋₄₂ was increased in AUD vm-PFC by 31%. N=17/group. t-test. *p<0.5 **p<0.01 ***p<0.001. Males-open circles, Females-closed circles.

FIGS. 2A-2T. Ethanol and obesity disrupt autophagic flux and lysosomal function in 3×Tg-AD mice. Ethanol (2A) increased P62 by 20%, (2B) reduced Beclin-1 by 14%, (2C) increased LC3 by 20%, and (2D) increased LAMP1 by 26% in 3×Tg-AD cortex. N=15 control, 18 ethanol. *p<0.05, **p<0.01, ****p<0.0001, t-tests. Males-open circles, females-closed circles. Obesity (2E) increased P62 by 33% (2F) reduced Beclin 21%, and (2G) reduced LC3 by 12.5% in 3×Tg-AD cortex. (2H) No change in LAMP1 was seen in obese 3×Tg-AD cortex. N=6 control, 7 diet-induced obesity. **p<0.01, ***p<0.001, t-tests. (2I) LAL (LIPA) gene expression was reduced by both EtOH and obesity. (2J) Reductions in LAL protein caused by ethanol (19%) and obesity (10%). Both ethanol (2K) and obesity (2L) reduced TFEB protein in 3×Tg-AD cortex (ethanol-20%, obesity-15.4%). *p<0.05, t-test. (2M) Reduced expression of lysosome acidifying v-ATPases genes by both ethanol and obesity. (2N) Lysosomal acidification in HEBSC slice cultures was reduced by ethanol (100 mM, 4 days) by (2O) 50% as measured by LysoTracker red. (2P-2S) Altered autophagy and reduced LAL in post-human AUD vm-PFC/BA25 in two cohorts. (2P) LC3-II was reduced by ~20% in AUD vm-PFC and (2Q) was correlated negatively with lifetime alcohol use and AUDIT score. (2R) In human cohort A, a reduction in LAL was found in AUD by Western blot that approach statistical significance. p=0.055. (2S, 2T) IHC on subjects from cohort B found a 50% reduction in LAL in human AUD vm-PFC. **p<0.01, paired t-test.

FIGS. 3A-3R. NLL levels predict intraneuronal Aβ and are enhanced by proinflammatory microglia. (3A) Triple immunofluorescence (IF) for cytosolic lipid (LipidSpot, green), LAMP1 (red), and Aβ (blue). Normal chow and water gavage groups were combined when no differences were present. Scale bar: 25 μm. (3B) Cytosolic lipid was increased by obesity (FCX: 80%, ENT: 120%) and EtOH (FCX: 38%, ENT: 80%). Open circles: males, filled circles: females. (3C) Increased lipid within Aβ+ neurons in FCX (obesity: 46%, ethanol: 75%) and ENT (obese: 2.8-fold, ethanol: 2.2-fold). (3D) Increased lysosomal lipid in Aβ+ neurons in FCX (obese: 79%, ethanol: 75%) and ENT (obese: 3.8-fold, ethanol 3.3-fold). (3E) Increased lysosomes in Aβ+ neurons with ethanol (2-fold FCX and ENT) and obesity (ENT: 76%). (3F) Increased intraneuronal Aβ in FCX (obesity: 56%, ethanol: 52%) and ENT (obesity: 2.1-fold, ethanol: 59%). Extra-lysosomal cytoplasmic levels of Aβ were increased in (3G) FCX in (obese: 61%, ethanol: 1.8-fold) and (3H) ENT (obese: 2.2-fold, ethanol: 1.9-fold). (3I) Reduction of lysosomal:extra-lysosomal cytoplasmic (lyso:cyto) Aβ ratios. (3J) High magnification image showing increased NLL in Aβ+ neurons. Scale: 10 μm. Strong positive correlations of NLL with intraneuronal Aβ in (3K) FCX and (3L) ENT with obesity and ethanol. (3M, 3N) Ethanol-induced increases in cytosolic lipid in HEBSCs was prevented by microglial inhibition (hM4di). (3O, 3P) Chemogenetic inhibition of proinflammatory microglia (CX3CR1Cre^{ERT2}.hM4di) prevented ethanol-induced (3P) microglial activation, (3Q) increases in total cytosolic lipid and (3R) NLL accumulation in the ENT. *p<0.05, **p<0.01, ***p<0.001, ****p<0.0001, Dunnett's post-test. #p<0.05, ##p<0.01, ###p<0.001, ####p<0.0001, Sidak's post-test.

FIGS. 4A-4S. Loss of neuronal LAL and accumulation of neuronal lysosomal lipid with aging in wild-type mice. (4A) Representative images of cytosolic lipid (LipidSpot), lysosomes (LAMP1), and neurons (MAP2) at 3, 12, and 20 months of age in frontal cortex (FCX). Higher magnification images of (4B) Neuronal lipid, (4C) lysosomal lipid, and (4D) neuronal lysosomal lipid at 20 months. Profound aging-related increases in FCX (4E) neuronal lipid, (4F) lysosomal lipid, and (4G) NLL, as well as ENT (4H) neuronal lipid, (4I) lysosomal lipid, (4J) and NLL from 3 to 20 months. 2-way ANOVAs, *p<0.05, **p<0.01, ***p<0.001, ****p<0.0001, Dunnett's post-test. Increased expression of (4K) PLIN3, 1-way ANOVA F_{2,48}=4.85, p<0.02, and (4L) PLIN4, F_{2,49}=4.85, p<0.02 and (4M) Srepb2 expression increases with age. F_{2,42}=8.45, p<0.001. *p<0.05, **p<0.01, ***p<0.001, Sidak's post-test. (4N-4O) Images of total and neuronal (within MAP2/green) LAL (red) with a 30% loss of neuronal LAL in ENT with age. (4P-4Q) In WT mice, a 30% increase in total Aβ (6E10) was found in ENT from 3 to 20 months. (4R) Total ENT Aβ was positively correlated with neuronal lysosomal lipid in WT ENT (4S) Negative correlation between Aβ and neuronal LAL. Open circles: males, filled circles: females.

FIGS. 5A-5DD. Loss of neuronal LAL drives Aβ accumulation. (5A, 5B) Loss of neuronal LAL precedes Aβ accumulation in AD mouse FCX and ENT. (5C, 5D) 3×Tg-AD regions where FCX had the highest levels of LAL and the lowest levels of Aβ, ENT had intermediate levels of both, and subiculum (SUB) had Aβ plaques and low levels of LAL. (5E-5H) Both ethanol and obesity caused a loss of total and neuronal LAL. (5I, 5J) Strong negative correlations were seen between Aβ₁₋₄₂ and LAL all subjects and regions with both ethanol and obesity. (5K-5R) Ex vivo studies using hippocampal-entorhinal brain slice cultures (HEBSC) from adult 3×Tg-AD mice found a causal relationship between LAL and Aβ. LAL inhibitor LAListat (LALi) caused a dose-dependent increase in (5K, 5L) lipid and (5M, 5N) intraneuronal Aβ in ENT region of HEBSC. Recombinant LAL blocked ethanol-induced increases in (5O, 5P) lipid and (5Q, 5R) intraneuronal Aβ. **p<0.01, ***p<0.0001, one-way ANOVA, Tukey's multiple comparisons test. (5S, 5T) Neuronal expression of shRNAs against LAL in 3×Tg-AD reduced neuronal LAL in ENT and SUB. Neuronal LAL knock-down (5U, 5V) increased intraneuronal Aβ and impaired (5W) learning, (5X) reversal learning, and (5Y) cognitive flexibility in the Morris water maze (MWM). Neuronal overexpression of LAL in 3×Tg-AD prior to ethanol treatment (5Z, 5AA) reduced NLL, (5BB, 5CC) lowered Aβ₁₋₄₂ in ENT and SUB, and (5DD) improved cognitive flexibility deficits.

FIGS. 6A-6N. Loss of LAL in humans with LOAD and across normal aging with polymerase pausing. (6A) LAL IHC finding a profound loss of LAL in human LOAD. In healthy control (HC) subjects, LAL was seen in cells with neuronal morphology, which was reduced in LOAD. (6B) 47% loss of LAL in ENT, (6C) 52% loss in CA1, (6D) trend toward a 31% reduction in subiculum, a (6E) 27% loss in the dentate gyrus, and a (6F) 56% loss on the vm-PFC. paired t-test ****p<0.0001, **p<0.01. Open circles-males, filled circles-females. Strong negative correlations between LAL and Aβ₁₋₄₂ staining were seen across all subjects in (6G) CA1 (R=−0.55, ****p<0.0001 and (6H) ENT (R=−0.4, *p=0.05). In healthy control subjects, LAL declined with age in (6I) CA1 and (6J) ENT. (6K) RT-PCR found a reduction in expression of the LAL gene LIPA. (6L) FOXO1 CHIP found increased LIPA promoter occupancy in LOAD. (6M) LIPA gene arrangement with arrows denoting sites assessed for Poll II localization. (6N) Active Poll II (pRpb1) CHIP found increased Poll II association with the LIPA promoter region and early exon 3 in LOAD with reduced localization at later exons 7 and 8. 2-way ANOVA LOAD main effect F_{1,68}=5.062, p=0.028. **p<0.01, Sidak's post-test.

FIGS. 7A-7DD. Neither chronic ethanol nor obesity had a significant impact on transgene expression, Aβ or Tau processing machinery. (7A) Ethanol had no effect on expression of the human APP (hAPP) transgene in cortex of 3×Tg-AD mice. (7B) Ethanol increased hAPP in male 3×Tg-AD hippocampus by 38%, with no change in females. Ethanol x sex, F_{1,32}=10.6, p=0.03. (7C) No effect of ethanol on hMAPT in cortex or hippocampus. (7D) Ethanol increased intrinsic cell death genes in 3×Tg-AD cortex and hippocampus. (7E) Ethanol had no impact on APP in WT mice. (7F) Ethanol had no effect on PSEN1 in 3×Tg-AD mice. (7G) Ethanol had no effect on BACE1 levels in 3×Tg-AD cortex. (7H, 7I) Ethanol reduced the inactive pSer9 isoform by 15.6% and increased active p-Tyr216 by 17% without significantly total GSK3β in 3×Tg-AD mice. No increases in cortical levels of (7J) CDK5, nor (7K) p-PKA were found. (7L) No effect of ethanol on PP2A in 3×Tg-AD cortex. p=0.28. (7M) Weights of 3×Tg-AD mice receiving Western diet across midlife. (7N) A main effect of sex on hAPP F_{1,9}=34.2, p=0.0002, with no effect of obesity. (7O) No change in hAPP expression in 3×Tg-AD hippocampus. (7P) No significant differences in PSEN1 or (7Q) BACE1 in obese mice. (7R) No change in hMAPT with obesity. (7S) No changes in p-PKA or (7T) CDK5 in hippocampus. (7U, 7V) Total and active GSK3β were unchanged in obese hippocampus, with a ~50% reduction of inactive p-Ser9 GSK3B. Obesity did not change cortical (7W, 7X) GSK3β (7Y) CDK5 or (7Z) p-PKA. N=7/group. (7AA) Expression of APP, MAPT, GSK3, and CDK5 were unchanged in human AUD hippocampus. (7BB-7CC) Increased GSK3β and GSK3a in human AUD vm-PFC. AUD Cohorts 1 and 2, N=17 age-matched healthy control and AUD subjects. (7DD) No change in BACE1 protein in human AUD vm-PFC. AUD Cohort A, N=9 age-matched healthy control and AUD subjects. *p<0.05, **p<0.01 paired t-tests.

FIGS. 8A-8Q. Midlife alcohol and obesity alter autophagy and LAL in rodents and humans. (8A) Ethanol increased p-mTOR (Ser2448) by ~2-fold in 3×Tg-AD cortex. (8B) Schematic summarizing reduced autophagic flux in 3×Tg-AD mice by ethanol. (8C) Schematic illustrating disruption of autophagic flux caused by midlife obesity. (8D) Trend toward reduced p-mTOR (Ser2448) in obese 3×-Tg-AD cortex. (8E-8H) Autophagy markers in WT cortex of 10 -mo mice treated with ethanol for 5 weeks. (8E) Ethanol increased P62 protein levels in females by 33%, *p=0.018, and did not alter levels in males. (8F) Main effect of ethanol on Beclin-1 protein. F_{1,16}=11.00, **p=0.004. There was a significant decrease in Beclin-1 protein in males (12% reduction, **p=0.008) and a trend toward a reduction in females (13%, p=0.08, t-test). (8G) Ethanol reduced LC3A/B by 12%, **p=0.0026, and (8H) increased LAMP1 levels in females (~33%, **p=0.0035). Obesity had no effect on (8I) P62 levels, (8J) Beclin, (8K) LC3, or (8L) LAMP1. (8M) Ethanol caused a loss of TFEB in female WT cortex. (8N) Reduction of lysosomal v-ATPase gene expression in the cortex of WT female mice treated with ethanol. *p<0.05, **p<0.01, ***p<0.001. (8O) Co-immunofluorescent labeling of TFEB and neuronal marker NeuN showing robust localization of TFEB in neurons. LAL was reduced in AUD (Cohort B) compared to age-matched controls as measured by IHC in (8P, 8Q) ENT-39%, CA1-27%, and subiculum-54% with no change was seen in the dentate. *p<0.05, **p<0.01 Paired t-tests.

FIGS. 9A-90. Lipid metabolic changes with ethanol or obesity in 3×Tg-AD and WT mice. (9A) Representative image of microglial lipid levels in 3×Tg-AD mice after chronic ethanol. Red: Iba-1, yellow-LipidSpot stain. (9B) Quantification of lipid within Iba-1+ found an ~2-fold increase in microglial lipid after ethanol treatment. (9C) The percentage of total lipid localized to microglia was slightly increased after ethanol. *p<0.05. (9D) Representative image of lysosomal (LAMP1) lipid staining in WT mice treated with water or ethanol. Scale bar: 25 μm. 9(E) Quantification found ethanol increased total lipid in WT mice. (9F) Ethanol increased the level of lysosomal lipid by ~2-fold. **p<0.01. (9G) A strong correlation was found between the level of lipid and Aβ in ENT of WT mice. R=0.9. (9H) Co-IF for p-tau 181 and LAMP1 in 3×Tg-AD CA1 after ethanol. Scale bar: 25 μm. (9I) Primarily cytosolic p-tau-181 was increased by ethanol, with lysosomal p-tau 181 was increased after ethanol. (9J) The p-tau 181 lyso:cyto ratio was not significantly reduced by ethanol. (9K) Neither ethanol nor obesity caused an upregulation in ER stress related genes. (9L) Gene expression of lipid droplet associated perilipins (PLINs), CGI-58, and lipid efflux transporters showed alterations in PLIN expression and reductions in ABCG1 and ABCA1 by both ethanol and obesity. Expression of major regulators of lipid metabolism and cytosolic lipases showed no robust pattern shared by both males and females after (9M) ethanol or (9N) diet-induced obesity in 3×Tg-AD mice. (9O) Ethanol had no robust effect on expression of key lipid metabolism genes or cytosolic lipases in WT mice. *p<0.05, **p<0.01, ***p<0.001, ****p<0.0001.

FIGS. 10A-10J. Age related changes in neuronal lipids and metabolic enzymes. Total cytosolic lipid increased dramatically in (10A) FCX. F_{2,29}=84.71, p<0.001 and (10B) ENT. No changes in (10C) total or (10D) neuronal lysosomes were found in FCX or ENT with age. (10E) total LAL in ENT declined ~30% from 3 to 20 months. In FCX, a reduction in (10F) total LAL was found, with (10G) sex-specific reductions in neuronal LAL. (10H) Across all subjects a negative correlation was found between neuronal lysosomal lipid and neuronal LAL. R=−0.36, *p<0.05. Expression of several lipid-regulatory genes in WT (10I) male and (10J) female cortex with age.

FIGS. 11A-11N. Modulation of lipid accumulation by TFEB, GLP/GIP, and LAL. (11A, 11B) The TFEB agonist genistein had no effect on ethanol-induction of lipid in 3×Tg-AD HEBSC. (11C, 11D) GLP/GIP agonist DA4-JC had minimal effect on lipid induced by ethanol in 3×Tg-AD HEBSC. PHP.eB.syn.shLAL viruses increased (11E, 11F) total cytosolic lipid and (11G, 11H) neuronal lipid in ENT and SUB of 3×Tg-AD mice. (11I) A strong positive correlation was found between intraneuronal Aβ and cytosolic lipid levels was found within Aβ-containing neurons. (11J) PHP.eB.syn. WPRE.LAL viruses increased LAL gene expression in cortex and hippocampus. (11K, 11L) Neuronal LAL overexpression prior to ethanol treatment reduced cytosolic lipid levels in ENT and SUB of 3×Tg-AD mice. (11M, 11N) Neuronal LAL overexpression had no effect on learning or reversal in the Morris water maze (MWM).

FIGS. 12A-12K. Human LOAD pathology. Healthy control (HC) and tissue from subjects with LOAD were assessed for AD amyloid pathology. Aβ₁₋₄₂ staining was increased in the (12A, 12B) ENT, (12C, 12D) CA regions of hippocampus, (12E, 12F) SUB, and (12G, 12H) dentate gyrus. (12I) FOXO1 protein levels were unchanged in LOAD by Western blot. (12J) A strong positive correlation was found between LIPA gene expression and Rpb2 promoter occupancy in the hippocampus of HC subjects but not in (12K) individuals with LOAD.

FIG. 13. Pathological Progression of AD with preclinical period for monitoring and prevention. A preclinical period prior to symptoms includes progressive Aβ and tau pathology. Adapted from Long and Holtzman Cell 2018.

FIG. 14. Schema for synthesis of ¹⁸F-LAL-x PET ligands

FIGS. 15A-15B. Generation of brain penetrant ¹⁸F-LAL-x. (FIG. 15A) labeling of ¹⁸F-LAL-x1 (FIG. 15B) in vivo study finding brain penetrance of ¹⁸F-LAL-x1 in WT adult mice.

FIG. 16. ¹⁸F-LAL-x retains its functional activity in brain slice culture.

FIG. 17. ¹⁸F-LAL-x measures loss of LAL in AD mouse brain compared to control (10 months of age).

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4^{th} Ed. (Cold Spring Harbor, NY, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### General Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together.

As used herein, the term “alkyl”, used either alone or in compound words such as “haloalkyl” includes straight-chain or branched alkyl, such as methyl, ethyl, n-propyl, i-propyl, or the different butyl, pentyl or hexyl isomers, etc. For example, alkyl refers to a straight or branched chain hydrocarbon containing from 1 to 5 carbon atoms, which can be referred to as a C1-C5 alkyl.

“Cyano” as used herein refers to a —CN group.

“Amide” as used herein alone or as part of another group refers to a —C(O)NRaRb radical, where Ra and Rb are any suitable substituent such as alkyl, cycloalkyl, alkenyl, alkynyl, alkoxy or aryl.

All chiral, diastereomeric, racemic, and geometric isomeric forms of a structure are intended, unless specific stereochemistry or isomeric form is specifically indicated. All processes used to prepare compounds and intermediates made therein are encompassed by the present disclosure. All tautomers of shown or described compounds are also encompassed by the present disclosure.

When any variable (e.g., Ri) occurs more than one time in any constituent or formula for a compound, its definition at each occurrence is independent of its definition at every other occurrence. Thus, for example, if a group is shown to be substituted with one or more Ri moieties, then Ri at each occurrence is selected independently from the Markush group recited for Ri. Also, combinations of substituents and/or variables are permissible, but only if such combinations result in stable compounds within a designated atom's normal valency.

As used herein, a “radioisotope” refers to a chemical element with unstable nuclei that releases excess energy by emitting radiation, for example, in the form of alpha, beta, and gamma rays, as it breaks down. A radioisotope may be synthetic or natural. Example radioisotopes include, but are not limited to, fluorine-18, carbon-11, nitrogen-13, gallium-68, and oxygen-15.

The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “tropism” as used herein refers to preferential but not necessarily exclusive entry of the vector (e.g., virus vector) into certain cell or tissue type(s) and/or preferential but not necessarily exclusive interaction with the cell surface that facilitates entry into certain cell or tissue types, optionally and preferably followed by expression (e.g., transcription and, optionally, translation) of sequences carried by the vector contents (e.g., viral genome) in the cell, e.g., for a recombinant virus, expression of the heterologous nucleotide sequence(s).

The term “tropism profile” refers to the pattern of transduction of one or more target cells, tissues and/or organs. Representative examples of chimeric AAV capsids have a tropism profile characterized by efficient transduction of cells of the central nervous system (CNS) with only low transduction of peripheral organs (see e.g., U.S. Pat. No. 9,636,370 McCown et al., and US patent publication 2017/0360960 Gray et al). Vectors (e.g., virus vectors, e.g., AAV capsids) expressing specific tropism profiles may be referred to as “tropic” for their tropism profile, e.g., neuro-tropic, liver-tropic, etc.

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide,” “nucleic acid,” or “nucleotide sequence” may be of RNA, DNA or DNA-RNA hybrid sequences (including both naturally occurring and non-naturally occurring nucleotides) but is preferably either a single or double stranded DNA sequence.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” (or grammatical equivalents) a cell, it is meant that the cell is at least partially separated from at least some of the other components in the starting material. e.g. biological sample.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease and encompass any type of prophylactic treatment to reduce the incidence of the condition or delay the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “heterologous nucleotide sequence” or “heterologous nucleic acid,” with respect to a virus or other vector, is a sequence or nucleic acid, respectively, that is not naturally occurring in the virus or other vector. Generally, the heterologous nucleic acid or nucleotide sequence comprises an open reading frame that encodes a polypeptide and/or a nontranslated RNA.

A “vector” refers to a compound used as a vehicle to carry foreign genetic material into another cell, where it can be replicated and/or expressed. A vector containing foreign or heterologous nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to as transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “viral vector” and “delivery vector” (and similar terms) in a specific embodiment generally refers to a virus particle that functions as a nucleic acid delivery vehicle, and which comprises the viral nucleic acid (i.e., the vector genome) packaged within the virion. Viral vectors according to the present invention may include chimeric AAV capsids according to the invention and can package an AAV or rAAV genome or any other nucleic acid including viral nucleic acids. Alternatively, in some contexts, the terms “viral vector” and “delivery vector” (and similar terms) may be used to refer to the vector genome (e.g., vDNA) in the absence of the virion and/or to a viral capsid that acts as a transporter to deliver molecules tethered to the capsid or packaged within the capsid.

The term “template” or “substrate” is used herein to refer to a polynucleotide sequence that may be replicated to produce the viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, the term “amino acid” encompasses any naturally occurring amino acids, modified forms thereof, and synthetic amino acids, including non-naturally occurring amino acids. Alternatively, the amino acid can be a modified amino acid residue or can be an amino acid that is modified by post-translation modification (e.g., acetylation, amidation, formylation, hydroxylation, methylation, phosphorylation or sulfonation). The non-naturally occurring amino acid can be an “unnatural” amino acid as described by Wang et al., (2006) Annu. Rev. Biophys. Biomol. Struct. 35:225-49.

A “functional fragment” of a polypeptide or protein, as used herein, means a portion of a larger polypeptide that substantially retains at least one biological activity normally associated with that polypeptide (e.g., wild-type protein or fragment thereof). In particular embodiments, the “functional” polypeptide or “functional fragment” substantially retains all of the activities possessed by the unmodified polypeptide (e.g., wild-type protein or fragment thereof). By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as binding activity can be measured using assays that are well known in the art and as described herein.

The term “fragment,” as applied to a peptide, will be understood to mean an amino acid sequence of reduced length relative to a reference peptide (e.g., wild-type protein) or amino acid sequence and comprising, consisting essentially of, and/or consisting of an amino acid sequence of contiguous amino acids identical to the reference peptide or amino acid sequence. Such a peptide fragment according to the invention may be, where appropriate, included in a larger polypeptide of which it is a constituent. In some embodiments, such fragments can comprise, consist essentially of, and/or consist of peptides having a length of at least about 5, 10, 15, 20, 25, 30, 35, 46. 50, 55, 60, 65, 70, 75, 80, 85, 90, 95 or 100 or more consecutive amino acids of a peptide or amino acid sequence according to the invention.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

As used herein, the term “derivative” is used to refer to a polypeptide which differs from a naturally occurring protein or a functional fragment by minor modifications to the naturally occurring polypeptide, but which substantially retains the biological activity of the naturally occurring protein. Minor modifications include, without limitation, changes in one or a few amino acid side chains, changes to one or a few amino acids (including deletions, insertions, and/or substitutions) (e.g., less than about 20, 15, 10, 9, 8, 7, 6, 5, 4, 3, or 2 changes), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol.

The term “substantially retains,” as used herein, refers to a fragment, derivative, or other variant of a polypeptide that retains at least about 50% of the activity of the naturally occurring polypeptide (e.g., binding to recognition sequence), e.g., about 60%, 70%, 80%, 90% or more.

The invention relates to the discovery that dysfunctional lipophagy promotes early Alzheimer's Disease (AD) pathology, and novel lysosomal acid lipase (LAL) positron emission tomography (PET) ligands. AD involves the accumulation of intraneuronal amyloid early in the disease course; by studying two common modifiable risk factors for AD, heavy alcohol use and obesity during midlife, the experiments show increased intraneuronal Aβ₁₋₄₂ in cortex and hippocampus without altering Aβ processing enzymes or gene expression, and reduced levels of LAL with subsequent lipid accumulation in neuronal lysosomes. As described herein, neuronal lysosomal lipid correlated positively with intraneuronal Aβ₁₋₄₂ in AD mice and increased greatly with age in wild-type mice, while neuronal LAL declined. Loss of LAL promoted intraneuronal Aβ₁₋₄₂ ex-vivo, while recombinant LAL prevented amyloid accumulation. In human AD brain, LAL was lost across brain regions and declined with age in healthy subjects. Together, this work implicates LAL loss early in AD progression and presents enhancing LAL activity as a preventative strategy for AD.

In an embodiment, a LAL PET ligand according to:

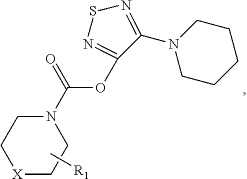

is provided, wherein X is C, CH, CH₂, CR₂, S, N, or O, wherein R₁ can be substituted at one or more positions on the piperidyl ring and each R₁ is independently a C₁-C₅ alkyl, C₁-C₅ cyano, C₁-C₅ amide group, a linker (e.g., a PEG linker or an arene-containing linker), or a halogen, and comprises a radioisotope, and wherein R₂ comprises a radiolabel. R₁ groups may be further substituted, including with ether, alcohol and/or carboxyl groups along the C₁-C₅ chain. Example arene-containing linkers include, but are not limited to, bis(phenylethynyl) arene linkers. See, Ferger et al., Chem. Eur. J. 2021, 27, 5142; Tumir et al., Molecules 2023, 28 (11)). A PEG linker can comprise —(CH2CH2O)x—, wherein x is an integer of 1, 5, 10, 25, or 50 and may be chemically functionalized, for example, with amine, azide, amine, TCO, alkyne, TCO, NHS ester, and maleimide. In some embodiments, the radioisotope is selected from fluorine-18, carbon-11, nitrogen-13 and oxygen-15. In some embodiments, the radiolabel can be fluorine-18, carbon-11, nitrogen-13 and oxygen-15, carbon-14, hydrogen-3 (tritium), or sulfur-35. The halogen can be F, Cl, Br, I, At, or Ts, and may optionally be a stable or radioactive isotope, for example, bromine-81, bromine-79, chlorine-37, chlorine-36, chlorine-35, iodine-127, iodine-129, fluorine-19, fluorine-23, astatine-215, astatine-217, astatine-218, astatine-219.

In some embodiments, the LAL PET ligand is according to

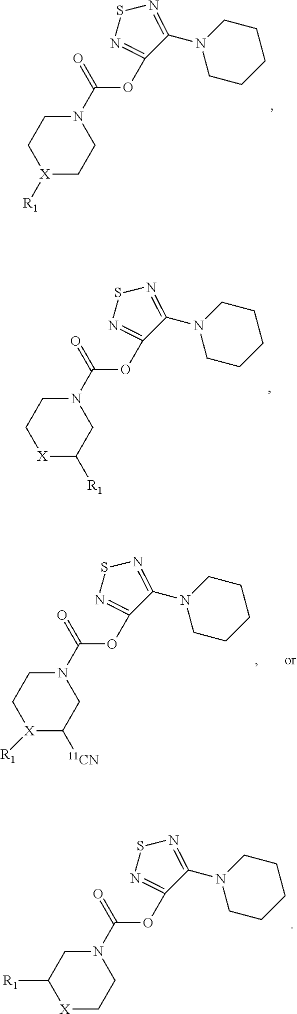

In some embodiments, the LAL PET ligand is selected from

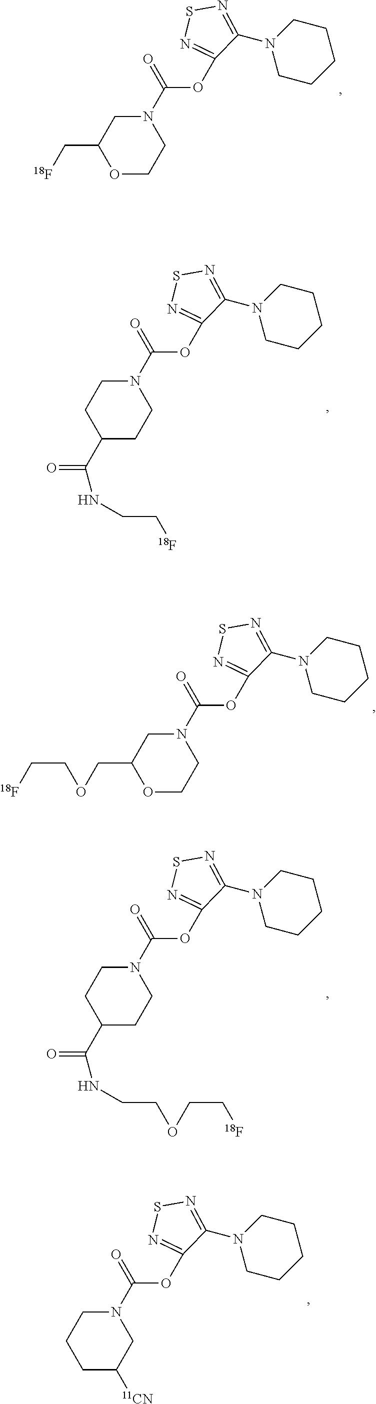

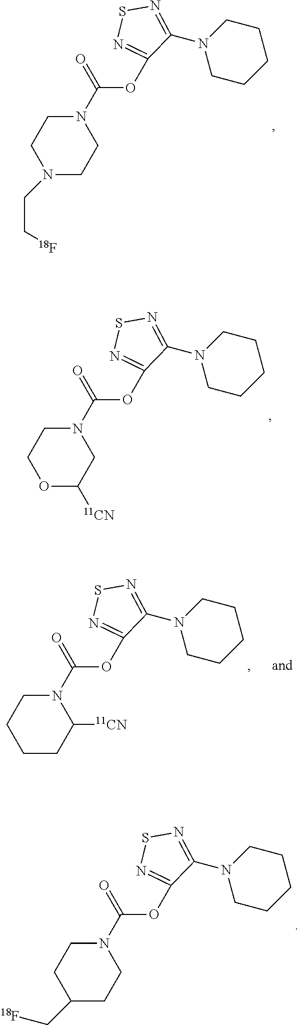

An aspect of the invention relates to a composition comprising the LAL PET ligand and another component, e.g., a suitable carrier. In an embodiment, the LAL PET ligand is provided in an effective amount in the composition. It is understood that the compositions of this invention can comprise, consist essentially of, or consist of any of the LAL PET ligands described herein in any combination and in any ratio relative to one another. Furthermore, by “two or more” is meant 2, 3, 4, 5, 6, 7, 8, 9, 10, etc., up to a total number of LAL PET ligands of this invention. A composition comprising a LAL PET ligand of the invention can be provided in a pharmaceutically acceptable carrier, wherein the pharmaceutical composition is administered in an effective amount, optionally with a pharmaceutically acceptable carrier.

In some embodiments, the LAL PET ligands can be used in the methods of one or more aspects of the present invention. The LAL PET ligands can be useful in measuring the amount and/or activity of lysosomal lipid accumulation or LAL in the brain of the subject, in a target cell, or in a sample from the subject. The LAL PET ligands can be useful in measuring the activity of lysosomal lipid accumulation or LAL in the brain of the subject, in a target cell, or in a sample from the subject. In some embodiment, the amount and activity of lysosomal lipoid accumulation or LAL is measured. In some embodiments, the LAL PET ligands can be used in methods of assessing the risk of developing AD or ADRD. The LAL PET ligands also find use in a method of diagnosing AD or ADRD in a subject.

In an embodiment, a method of assessing the risk of developing AD or ADRD in a subject is provided. The method can comprise the steps of: (a) measuring the amount and/or activity of lysosomal acid lipase (LAL) in the subject; (b) comparing the amount and/or activity of LAL measured in (a) with a threshold amount and/or activity of LAL; and (c) identifying an increased risk of developing AD based on the amount and/or activity of LAL measured in (a) being lower than the threshold amount and/or activity of LAL. The method can further comprise a step of administering a therapy to treat the subject based on the diagnosis or identification of the risk of developing AD/ADRD.

In some embodiments, the method of assessing the risk of developing AD or ADRD in a subject further comprises (d) measuring the amount and/or activity of lysosomal lipid accumulation in the brain of the subject, and (e) comparing the amount and/or activity of the lipid accumulation measured in (d) with a threshold amount and/or activity of lipid accumulation, thereby diagnosing or identifying an increased risk of developing AD/ADRD based on the amount and/or activity of the lipid accumulation measured in (d) that is higher than the threshold amount and/or activity of the lipid accumulation.

In some embodiments, the method of diagnosing AD or ADRD in a subject further comprises (d) measuring the amount and/or activity of lysosomal lipid accumulation in the brain of the subject, and (e) comparing the amount and/or activity of the lipid accumulation measured in (d) with a threshold amount and/or activity of lipid accumulation, thereby diagnosing or identifying an increased risk of developing AD/ADRD based on the amount and/or activity of the lipid accumulation measured in (d) that is higher than the threshold amount and/or activity of the lipid accumulation. The threshold amount and/or activity of the lipid accumulation measured in (d) can be determined by analysis of data collected from sample populations of healthy subjects and subjects with AD/ADRD. The threshold amount and/or activity of the lipid accumulation measured in (d) can be determined by analysis of data collected from sample populations of healthy subjects and subjects with AD/ADRD.

In an embodiment, a method of diagnosing AD or ADRD in a subject is provided. The method can comprise the steps of: (a) measuring the amount and/or activity of LAL in the subject; (b) comparing the amount and/or activity of LAL measured in (a) with a threshold amount and/or activity of LAL; and (c) diagnosing AD based on the amount and/or activity of LAL measured in (a) being lower than the threshold amount and/or activity of LAL. The method can further comprise a step of administering a therapy to treat the subject based on the diagnosis or identification of the risk of developing AD/ADRD.

The threshold amount and/or activity of LAL, for example, in step (b) of the methods, can be used when comparing the amount and/or activity of LAL measured. The threshold amount of LAL can be determined by analysis of data collected from sample populations of healthy subjects, subjects with AD/ADRD or the threshold amount and/or activity of LAL may be determined by measurement of LAL amount and/or activity in the subject at an earlier time. For example, LAL levels from a longitudinal plasma biobank from the 1980s in which some patients now have AD, can be utilized in order to determine threshold plasma LAL levels.

The threshold amount and/or activity of lipid accumulation, for example, in step (b) of the methods, can be used when comparing the amount of lipid accumulation measured. The threshold amount of lipid accumulation can be determined by analysis of data collected from sample populations of healthy subjects, subjects with AD/ADRD or the threshold amount of lipid accumulation may be determined by measurement of lipid accumulation amount and/or activity in the subject at an earlier time. For example, lipid accumulation levels from a longitudinal plasma biobank from the 1980s in which some patients now have AD, can be utilized in order to determine threshold plasma lipid accumulation levels.

In some embodiments, the methods can further comprise calculating a risk factor (P1) based on the amount and/or activity of LAL measured in a subject and the threshold amount and/or activity of LAL, wherein a higher range of P1 identifies the subject as having AD/ADRD or having an increased risk of developing AD/ADRD. In an embodiment, a higher risk factor P1 comprises a LAL lower in the subject than that of healthy patients in a threshold value. The methods can further comprise calculating a risk factor, P2, based on the amount of the lipid accumulation measured in (d) and the threshold amount of the lipid accumulation measured in (e), wherein a higher range of P2 identifies the subject as having AD or having an increased risk of developing AD/ADRD. In some embodiments, a higher range of P2 is a percent increase, for example, at least 1%, 2%, 3%, 4%, 5%, 6%, 7%, 8%, 9%, 10%, 11%, 12%, 13%, 14%, 15%, 16%, 17%, 18%, 19%, 20% or more. In an embodiment, a higher risk factor P2 comprises a lipid accumulation higher in the subject than that of healthy patients of a threshold value.

In some embodiments, measuring the amount and/or activity of LAL or lysosomal lipid accumulation in the brain of the subject in step (d) is performed on cortex, hippocampus, parahippocampal regions, entorhinal regions, brainstem regions, or a combination thereof.

Methods of the invention disclosed herein can comprise methods of measuring LAL in a sample from a human subject, comprising measuring the amount and/or activity of LAL in a blood sample, cerebrospinal fluid (CSF) sample, brain sample, or cheek swab, or a combination thereof. In some embodiments, a method of determining one or more LAL mutations in a human subject is provided, comprising gene sequencing LAL in a sample from a human subject to thereby identify one or more LAL mutations in the sample, optionally wherein the sample is a blood sample, CSF sample, brain sample, or cheek swab, or a combination thereof.

In some embodiments measuring the amount and/or activity of LAL in the subject, for example in step (a) of the methods, is performed on a blood sample, CSF sample, brain sample, or cheek swab, or a combination thereof. In some embodiments, the sample comprises peripheral cells. In an embodiment, the sample is processed to provide isolated cells. In an embodiment, the measuring LAL is performed in IPSC derived neurons from the subject.

In some embodiments, measuring the amount and/or activity of LAL or lysosomal lipid accumulation can be performed by radio-imaging, immunostaining, immunofluorescence, fluorometry, flow cytometry, mass spectrometry, or a combination thereof. Other exemplary methods of measuring the amount and/or activity of LAL include DNA or RNA sequencing methods, which can include evaluation of LAL loss of function mutations or loss of LAL gene expression. In one embodiment, the radio-imaging of LAL is positron emission tomography (PET) which may be performed by detecting a radio-labeled LAL ligand. In an embodiment, the LAL ligand is an antibody, for example, commercially available LAL rabbit anti-Human polyclonal antibody. In an embodiment, the LAL ligand can be according to:

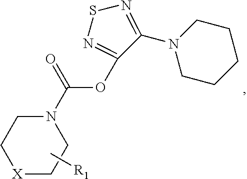

wherein X is C, CH, CH₂, CR₂, S, N, or O, wherein R₁ can be substituted at one or more positions on the piperidyl ring and each R₁ is independently a C₁-C₅ alkyl, C₁-C₅ cyano, C₁-C₅ amide group, a linker (e.g., a PEG linker or an arene-containing linker), or a halogen, and comprises a radioisotope, and wherein R₂ comprises a radiolabel. In an embodiment, the radioisotope is selected from fluorine-18, carbon-11, nitrogen-13 and oxygen-15. The LAL PET ligand can be according to:

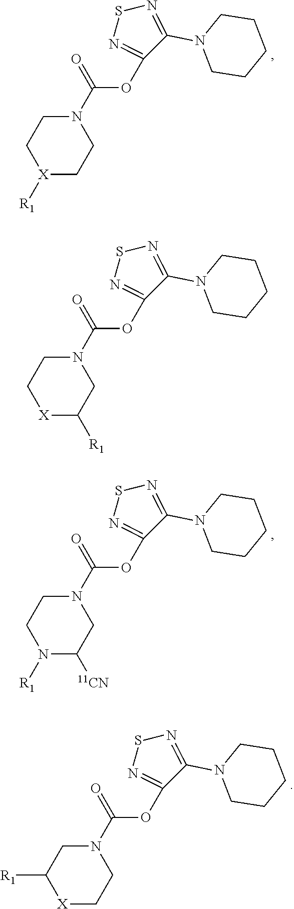

In some embodiments, the LAL PET ligand is selected from

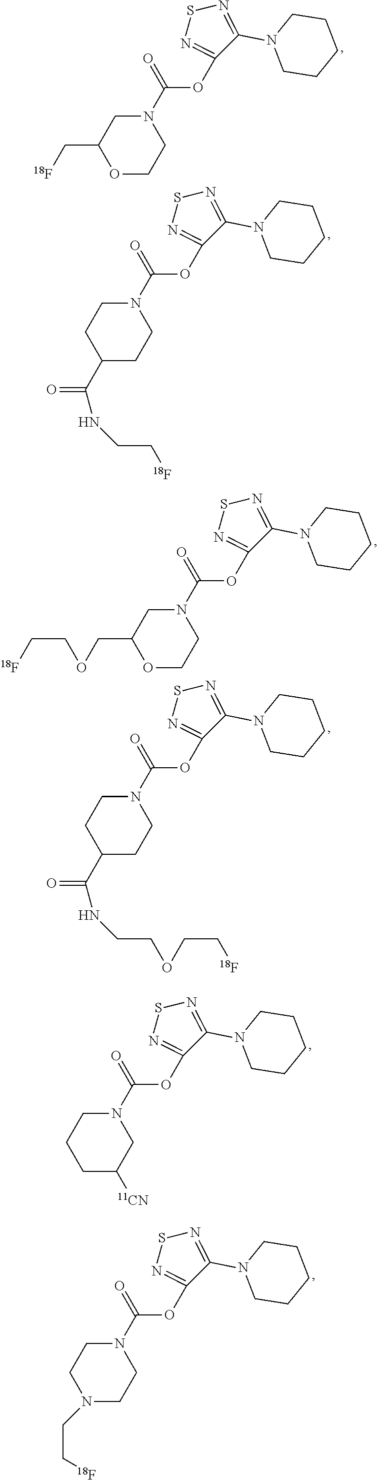

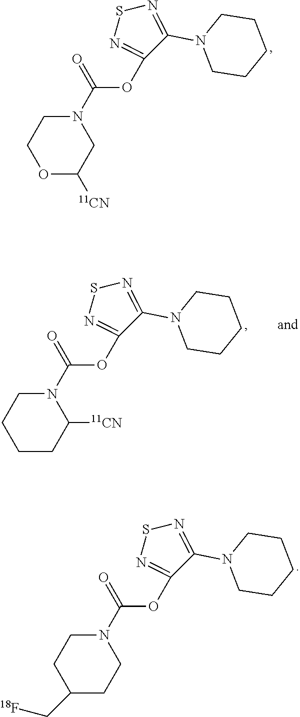

Exemplary assays for LAL also include measuring LAL in dried blood spots, including traditional fluorometry-based and UPLC-MS/MS-based assays. See, e.g., Masi. Et al., Clin Chem; 64(4): 690-696 (2018); Lucaks et al., Clin. Chim. Acta, 471, 201-205 (2017); Hong, et al., Molecular Genetics and Metabolism Reports, 33, 100935 (2022).

In an embodiment, the methods of assessing the risk of developing AD or ADRD and/or diagnosing AD or ADRD in a subject can further comprise administering one or more standard AD/ADRD diagnostic tests. Exemplary standard diagnostic tests include cognitive functional and behavioral tests, such as Ascertain Dementia 8 (AD8), Functional Activities Questionnaire (FAQ), Mini-Cog, Mini-Mental State Exam (MMSE), Montreal Cognitive Assessment (MoCA), and Neuropsychiatric Inventory Questionnaire (NPI-Q). Additional standard diagnostics include medical history, physical exam, neurological exam, depression screen and mood assessment, brain imaging such as with magnetic resonance imaging (MRI) or computed tomography (CT), CSF levels of multiple markers such as tau, beta-amyloid, neurofilament light (NfL), and computerized cognitive tests and devices such as Automated Neuropsychological Assessment Metrics (ANAM), Cambridge Neuropsychological Test Automated Battery (CANTAB Mobile®), CognICA, Cognigram, Cognivue, and Cognision. In an embodiment, the standard test comprises the assessment of neurodegeneration biomarkers of AD/ADRD. Exemplary neurodegeneration biomarkers that can be used in the test include Aβ₁₋₄₂, phosphorylated tau (p-tau), PPARγ, IRF1, LxRβ, CEBPα, LxRα, or a combination thereof. In embodiments, the subject is a mammalian subject, for example, a human subject.

In some embodiments, the step of administering a therapy to treat the subject based on the diagnosis or identification of the risk of developing AD/ADRD is selected from a group of enzyme replacement therapy, lipid-lowering drugs, and dietary manipulation, for example, with increased caprylic acid, coconut oil, or a combination thereof. In an embodiment, therapies for AD treatment can include Aducanumab (Aduhelm™) or Lecanemab (Leqembi™) including for AD subjects with mild cognitive impairment (MCI) or mild dementia. Medications for treatment of ADRD can include Donepezil (Aricept®), Galantamine (Razadyne®), Rivastigmine (Exelon®), Memantine (Namenda®), and Memantine+Donepezil (Namzaric®).

In an embodiment, the therapy to treat the subject is enzyme replacement therapy and comprises administering LAL into the brain or central nervous system (CNS) of the subject. Exemplary LAL protein sequences include Reference sequence NP_001121077.1, incorporated herein by reference in its entirety. In an embodiment, the enzyme replacement therapy comprises systemically administering LAL to the subject. In an embodiment, the LAL is formulated in a vesicle or nanoparticle. The LAL can be sebelipase α/rLAL.

The enzyme replacement therapy can comprise administering a polynucleotide encoding LAL into the brain or CNS of the subject or it may be systemically administered. An exemplary sequence of human LIPA (lipase A), the gene encoding LAL, is Accession No. NM_001127605, incorporated herein by reference in its entirety. Example transcript variants of LIPA encoding an LAL protein include NM_001127605.3 (transcript variant 1), NM_000235.4 (transcript variant 2), NM_001288979.2 (transcript variant 3), each incorporated herein by reference in its entirety. In an embodiment, the polynucleotide is provided in a delivery vector. The delivery vector may be a viral vector, for example, an adeno-associated virus (AAV) vector as described further herein.

In some embodiments, a method of treating a subject diagnosed with or at risk of developing AD/ADRD is provided, the method comprising: (a) performing the method described herein to determine whether the subject has AD/ADRD or has an increased probability of developing AD/ADRD; and (b) administering a therapy to the subject based on the results of (a).

A method of treating AD/ADRD in a subject in need thereof is provided, the method comprising administering LAL to the subject, thereby treating AD/ADRD.

In an embodiment, a method for slowing the progression of AD/ADRD in a subject in need thereof is provided. Slowing the progression is determined relative to a subject that has not received the treatment. The method for slowing the progression of AD/ADRD can comprise administering LAL to the subject, thereby slowing the progression of AD/ADRD. LAL can be provided as an enzyme therapy, or as a polynucleotide encoding LAL as described herein.

Methods of diagnosing AD/ADRD in a subject are provided. The method of diagnosing AD/ADRD can comprise the steps of (a) measuring the amount and/or activity of LAL in the subject; (b) comparing the amount and/or activity of LAL in the subject with the amount and/or activity of LAL in a sample population of healthy subjects and subjects with AD/ADRD; (c) performing one or more standard AD/ADRD diagnostic tests on the subject; and (d) diagnosing whether the subject has AD/ADRD based on the results of (b) and (c).

A further aspect of the invention relates to methods for monitoring the response to LAL therapy in a subject, comprising measuring the amount and/or activity of LAL one or more times (e.g., 1, 2, 3, 4, 5, or more times) in a subject undergoing LAL therapy to determine changes in the amount and/or activity of LAL over time (e.g., before and after the initiation of therapy, after repeated administrations of LAL therapy, etc.). In some embodiments, the subject is being treated for AD, ADRD, or LAL deficiency (LAL-D) disorder. The monitoring may be used to determine whether a therapeutically effective amount and/or activity of LAL has been achieved, which can, for example, inform the decision to increase, decrease, or terminate the LAL therapy. Measuring the amount and/or activity of LAL may involve use of the LAL ligands of the invention.

### Delivery Vectors

The compositions of the present invention can be used with delivery vectors, for example, a recombinant AAV, that comprises a polynucleotide encoding LAL.

The delivery vector may be any type of vector known to be useful for delivering a polynucleotide to a cell. In some embodiments, the delivery vector is a viral vector, e.g., a viral genome. Examples of viral vectors include, without limitation, an adeno-associated virus, retrovirus, lentivirus, poxvirus, alphavirus, baculovirus, vaccinia virus, herpes virus, Epstein-Barr virus, or adenovirus vector. As used herein, the term “gene delivery vector” refers to a delivery vector capable of delivering a gene to a cell or to a subject and expressing the gene in the cell or subject; the transgene delivery vector may utilize the delivery vectors described herein.

In some embodiments, the delivery vector is a non-viral vector. Examples of non-viral vectors include, without limitation, a plasmid, liposome, electrically charged lipid, nucleic acid-protein complex, or biopolymer. In an embodiment, a protein can be delivered in a non-viral vector, such as a nanoparticle or liposome.

Another aspect of the invention relates to a cell comprising the delivery vector of the invention. The cell may be in vitro or in vivo.

A further aspect of the invention relates to a composition comprising the delivery vector or the cell of the invention, e.g., a pharmaceutical composition comprising the gene delivery vector or the cell of the invention and a pharmaceutically acceptable carrier.

In some embodiments of the invention, the delivery vector is a parvovirus vector. The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

In some embodiments of the invention, the delivery vector is a parvovirus within the genus Dependovirus. The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers). A number of additional AAV serotypes and clades have been identified (see, e.g., Gao et al., (2004) J. Virol. 78:6381-6388), which are also encompassed by the term “AAV.”

The parvovirus particles and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73:939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-:375-383; Mori et al., (2004) Virol. 330:375; Muramatsu et al., (1996) Virol. 221:208; Ruffing et al., (1994) J. Gen. Virol. 75:3385; Rutledge et al., (1998). J. Virol. 72:309; Schmidt et al., (2008). J. Virol. 82:8911; Shade et al., (1986). J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

The term “AAV viral vectors” includes “chimeric” AAV nucleic acid capsid coding sequence or AAV capsid protein is one that combines portions of two or more capsid sequences. A “chimeric” AAV virion or particle comprises a chimeric AAV capsid protein.

Considerations for delivery of AAV to the brain and neurodegenerative diseases, are discussed in Fischell and Fishman, Front. Neurosci. 15:747726 (2021). doi: 10.3389/fnins.2021.747726, incorporated herein by reference. Example AAVs that are known to cross the BBB which may be used for the delivery of LAL include AAV9, AAVrh10, AAVhu.32, and AAVrh8.

In some embodiments, the delivery vector encodes an LAL enzyme or a nucleic acid encoding the LAL enzyme, including one that can substitute for a missing or defective protein in a subject.

The transgene delivery vectors may also comprise a nucleic acid that shares homology with and recombines with a locus on a host chromosome. This approach can be utilized, for example, to correct a genetic defect in the host cell.

It will be understood by those skilled in the art that the nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible. Exemplary neuronal promoters for use with AAVs include the human Synapsin promoter (hSyn1) and mouse PGK (mPGK).

Inducible expression control elements are typically advantageous in those applications in which it is desirable to provide regulation over expression of the nucleic acid sequence(s). Inducible promoters/enhancer elements for gene delivery can be tissue-specific or -preferred promoter/enhancer elements, and include neural tissue specific or preferred (including brain-specific or preferred), promoter/enhancer elements. Other inducible promoter/enhancer elements include hormone-inducible and metal-inducible elements or cell stress-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence(s) is transcribed and then translated in the target cells, specific initiation signals are generally included for efficient translation of inserted protein coding sequences. These exogenous translational control sequences, which may include the initiation codon (e.g., ATG) and adjacent sequences, can be of a variety of origins, both natural and synthetic.

The cell(s) into which the delivery vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle cells (e.g., skeletal muscle cells, cardiac muscle cells, smooth muscle cells and/or diaphragm muscle cells), dendritic cells, endothelial cells, germ cells, and the like. In representative embodiments, the cell can be any progenitor cell. As a further possibility, the cell can be a stem cell (e.g., neural stem cell, liver stem cell). Moreover, the cell can be from any species of origin, as indicated above. Furthermore, the cells may be dividing or non-dividing.

Embodiments of the invention may be performed in vitro or in vivo. One aspect of the present invention is a method of expressing a transgene in a cell in vitro, e.g., for research purposes or as part of an ex vivo method. The transgene delivery vector may be introduced into the cells at the appropriate amount, e.g., multiplicity of infection for a viral vector, according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10⁵ infectious units are introduced to the cell.

In particular embodiments, the cells have been removed from a subject, the delivery vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the delivery vectors can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the delivery vector are administered to the subject in a treatment effective or prevention effective amount in combination with a pharmaceutical carrier.

The delivery vectors are additionally useful in a method of delivering a nucleic acid to a subject in need thereof, e.g., to express a therapeutic polypeptide, e.g., LAL, or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The delivery vectors can also be used to produce a polypeptide of interest or functional RNA in a subject (e.g., using the subject as a bioreactor to produce the polypeptide or to observe the effects of the functional nucleic acid on the subject, for example, in connection with screening methods). The delivery vectors may also be employed to provide a functional nucleic acid to a cell in vitro or in vivo. Expression of the functional nucleic acid in the cell, for example, can diminish expression of a particular target protein by the cell. Accordingly, functional nucleic acid can be administered to decrease expression of a particular protein in a subject in need thereof.

Delivery vectors also find use in diagnostic and screening methods, whereby a nucleic acid of interest is transiently or stably expressed in a transgenic animal model.

The delivery vectors can also be used for various non-therapeutic purposes, including but not limited to use in protocols to assess gene targeting, clearance, transcription, translation, etc., as would be apparent to one skilled in the art. The delivery vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

The present invention further comprises a kit or kits to carry out the methods of this invention. A kit of this invention can comprise reagents, buffers, and apparatus for mixing, measuring, sorting, labeling, etc., as well as instructions and the like.

In some embodiments, the invention provides a kit for comprising LAL or a nucleic acid encoding LAL of the invention, and/or expression cassettes and/or vectors and/or cells comprising the same as described herein, with optional instructions for the use thereof.

### Pharmaceutical Formulations, Subjects, and Modes of Administration

Provided according to embodiments of the invention are compositions that include LAL or a polynucleotide encoding LAL. Also provided herein are pharmaceutical compositions comprising a LAL or a polynucleotide encoding LAL and optionally a delivery vector in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form. By “pharmaceutically acceptable” it is meant a material that is not toxic or otherwise undesirable, i.e., the material may be administered to a subject without causing any undesirable biological effects.

Administration of the LAL PET ligand, LAL or a polynucleotide encoding LAL to a human subject or an animal in need thereof can be by any means known in the art. Optionally, the delivery vector with the LAL is delivered in a treatment effective or prevention effective dose in a pharmaceutically acceptable carrier.

Injectables can be prepared in conventional forms, either as liquid solutions or suspensions, solid forms suitable for solution or suspension in liquid prior to injection, or as emulsions. Alternatively, one may administer the LAL or polynucleotide encoding LAL in a local rather than systemic manner, for example, in a depot or sustained-release formulation. The LAL or a polynucleotide encoding LAL, optionally within delivery vectors disclosed herein can be administered to the lungs of a subject by any suitable means, optionally by administering an aerosol suspension of respirable particles comprised of LAL or a polynucleotide encoding LAL, which the subject inhales. The respirable particles can be liquid or solid. Aerosols of liquid particles comprising the LAL or a polynucleotide encoding LAL may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the LAL or a polynucleotide encoding LAL may likewise be produced with any solid particulate medicament aerosol generator, by techniques known in the pharmaceutical art.

In certain embodiments, the LAL or a polynucleotide encoding LAL are administered to a subject in need thereof as early as possible in the life of the subject, e.g., as soon as the subject is diagnosed with a disease or disorder. In some embodiments, the methods are carried out on a newborn subject, e.g., after newborn screening has identified a disease or disorder. In some embodiments, methods are carried out on a subject prior to the age of 10 years, e.g., prior to 1, 2, 3, 4, 5, 6, 7, 8, 9, or 10 years of age. In some embodiments, the methods are carried out on juvenile or adult subjects after the age of 10 years. In some embodiments, the methods are carried out on a fetus in utero, e.g., after prenatal screening has identified a disease or disorder. In some embodiments, the methods are carried out on a subject as soon as the subject develops symptoms associated with a disease or disorder, e.g., AD/ADRD. In some embodiments, the methods are carried out on a subject before the subject develops symptoms associated with a disease or disorder, e.g., a subject that is suspected of or risk of having AD/ADRD or diagnosed as having a disease or disorder but has not started to exhibit symptoms.

The LAL or a polynucleotide encoding LAL may be administered to a subject by any route of administration found to be effective to regulate expression in the host cell. The most suitable route will depend on the subject being treated and the disorder or condition being treated. In some embodiments, the compositions are administered to the subject by a route selected from oral, rectal, transmucosal, intranasal, inhalation (e.g., via an aerosol), buccal (e.g., sublingual), vaginal, intrathecal, intraocular, intravitreal, intracochlear, transdermal, intraendothelial, in utero (or in ovo), parenteral (e.g., intravenous, subcutaneous, intradermal, intracranial, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye (e.g., by intrastromal, topical, intracameral, intravitreal, subconjunctival, suprachoroidal, sub-Tenon, retrobulbar, or subretinal administration), skeletal muscle, cardiac muscle, diaphragm muscle or brain (e.g., by intrathecal, intracerebral, intraventricular, intranasal, intra-aural, intra-ocular, or peri-ocular delivery administration)).

In particular embodiments, more than one administration (e.g., two, three, four or more administrations) may be employed to achieve the desired level of gene expression over a period of various intervals, e.g., daily, weekly, monthly, yearly, etc.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

The LAL or a polynucleotide encoding LAL can be administered to tissues of the CNS (e.g., brain, eye) and may advantageously result in effective distribution of the LAL and can optionally be provided in a delivery vector. Administration can be to any site in a subject, including, without limitation, the brain.

In general, the delivery vectors of the present invention can be employed to deliver a nucleic acid encoding a polypeptide or functional nucleic acid to treat and/or prevent any disease state for which it is beneficial to deliver a therapeutic polypeptide, e.g., LAL, or functional nucleic acid.

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, delivery vectors permit the treatment and/or prevention of genetic diseases.

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a polypeptide or polynucleotide including those described herein. As a further option, the subject can be a laboratory animal and/or an animal model of disease. Preferably, the subject is a human.

Dosages of the compositions to be administered to a subject depend upon the mode of administration, the disease or condition to be treated and/or prevented, the individual subject's condition, the composition to be delivered, and the like, and can be determined in a routine manner. Exemplary doses for achieving therapeutic effects with delivery vectors are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸-10¹⁵ transducing units.

In a representative embodiment, the invention provides a method of treating and/or preventing AD/ADRD in a subject in need thereof, the method comprising: administering a treatment or prevention effective amount of delivery vectors to a mammalian subject, wherein the delivery vector comprises LAL or polynucleotide encoding LAL. In particular embodiments, the delivery vectors can be administered to the brain or CNS as described elsewhere herein. In some embodiments, the delivery vectors can be systemically delivered to other tissues to treat and/or prevent a disorder.

In particular embodiments, the delivery vectors can comprise a secretory signal as described in U.S. Pat. No. 7,071,172. In an embodiment, the delivery vectors can comprise a lysosomal targeting signal, for example, a peptide-based glycosylation-independent lysosomal targeting (GILT) tag (Maga et al., J. Of Biol. Chem., 288, 3, 1428-1438 (2013) incorporated herein by reference in its entirety) or a mannose-6-phosphate glycan (Kang, et al., Scientific Reports 8:8730 (2018) incorporated herein by reference in its entirety).

In representative embodiments of the invention, the delivery vectors are administered to the CNS (e.g., to the brain or to the eye). The delivery vectors may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The delivery vectors may also be administered to different regions of the eye such as the retina, cornea and/or optic nerve.

The delivery vectors may be delivered into the cerebrospinal fluid (e.g., by lumbar puncture) for more disperse administration of the delivery vectors. The delivery vectors may further be administered intravascularly to the CNS in situations in which the blood-brain barrier has been perturbed (e.g., brain tumor or cerebral infarct).

The delivery vectors can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the LAL or polynucleotide encoding LAL, optionally within delivery vectors, are administered in a liquid formulation by direct injection (e.g., stereotactic injection) to the desired region or compartment in the CNS. In other embodiments, the LAL or polynucleotide encoding LAL, optionally within delivery vectors may be provided by topical application to the desired region or by intra-nasal administration of an aerosol formulation. Administration to the eye, may be by topical application of liquid droplets. As a further alternative, the LAL or polynucleotide encoding LAL may be administered as a solid, slow-release formulation. The LAL or polynucleotide encoding LAL can be provided in a solid, slow release formulation of delivery vectors as described, for example (see, e.g., U.S. Pat. No. 7,201,898).

Having described the present invention, the same will be explained in greater detail in the following examples, which are included herein for illustration purposes only, and which are not intended to be limiting to the invention.

### EXAMPLES

### Example 1

Alzheimer's disease (AD) is the most common form of dementia with over 90% of cases being sporadic/late-onset (LOAD). Though the etiology of LOAD is often unknown, risk factors such as heavy smoking or alcohol use, diabetes, hypertension, and obesity account for majority of risk. Though these exposures have diverse biological consequences, they result in the same pathological progression—accumulation of intraneuronal amyloid (Aβ) followed by extracellular Aβ plaque deposition, and accumulation of tau with aging. Therefore, identifying shared consequences of these exposures can reveal cellular drivers of LOAD. Several cellular functions have recently been implicated in LOAD including neuroinflammation, the endosomal and autophagosomal/lysosomal systems, and altered lipid metabolism. However, how these systems integrate with each other to result in emergence of LOAD in diverse etiological settings remains unknown.

Cellular and molecular consequences of two distinct midlife risk factors for LOAD were compared, heavy alcohol use and obesity. A recent nationwide retrospective study found that heavy alcohol use was the strongest modifiable risk factor for dementia and AD (HR~3 and 2 respectively) 5. It was recently reported that early life binge alcohol increased AD pathology in adulthood²⁰. This involved proinflammatory microglial activation, though the mechanism underlying neuronal amyloid accumulation was unknown. Using these two risk exposures to identify shared cellular deficits that underly LOAD pathogenesis, the following example found that the accumulation of neuronal lysosomal lipid (NLL) is a fundamental driver of early LOAD pathogenesis. This was caused by the loss of lysosomal acid lipase (LAL) expression, which was also found in normal aging in WT mice and healthy human brain. This phenomenon extended beyond these risk factors, with human LOAD subjects without heavy alcohol use or obesity showing widespread loss of LAL in brain, with transcriptional repression of LAL gene expression by pausing of the RNA polymerase II. This finding has significant implications for the understanding of disease progression, identification of at-risk individuals, and treatments to prevent or slow the progression of LOAD in the human population.

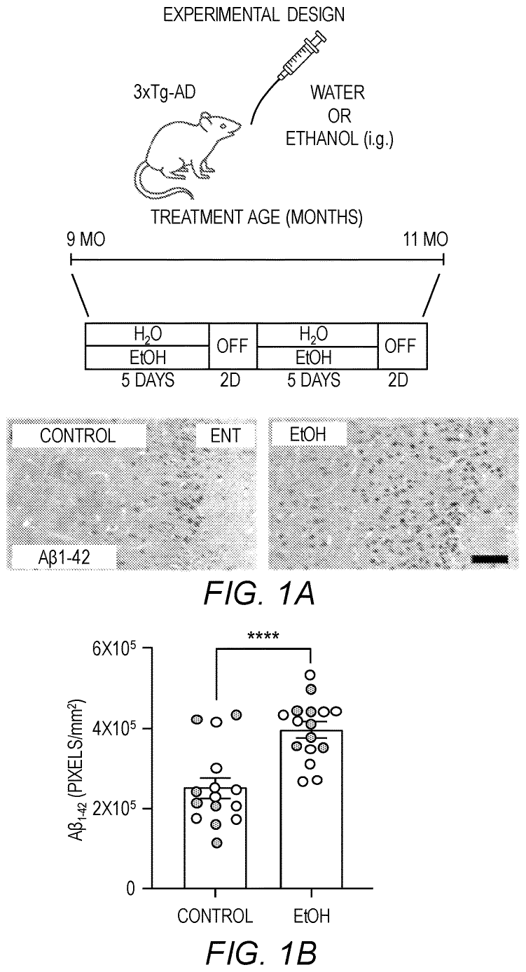

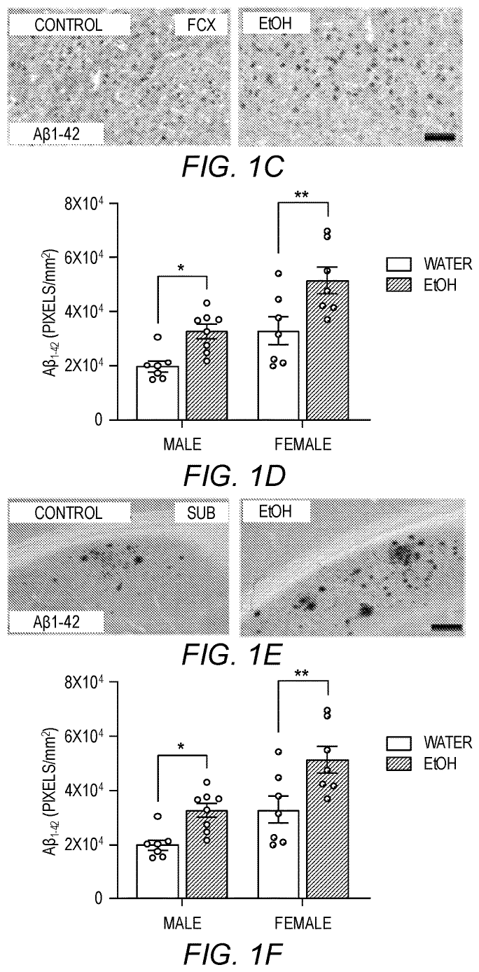

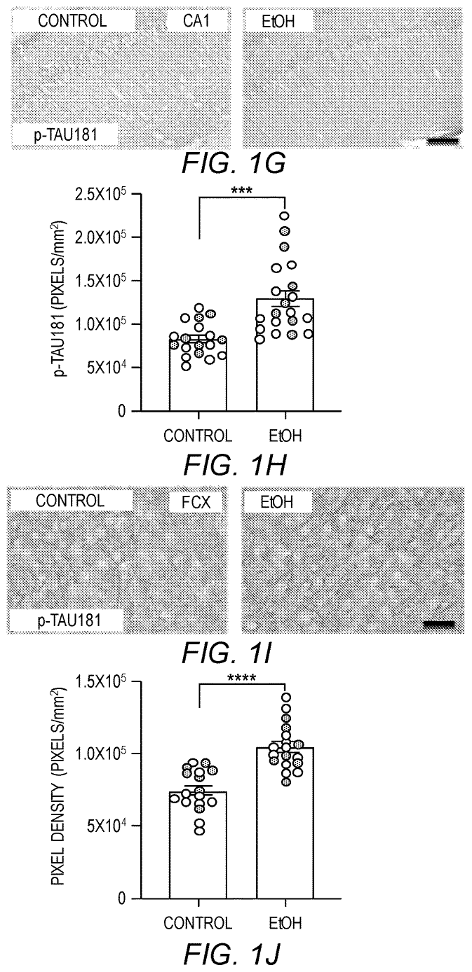

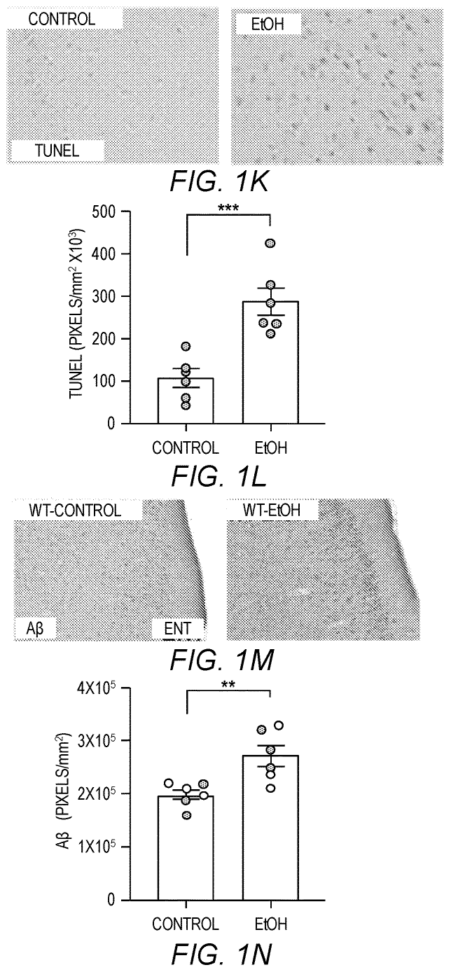

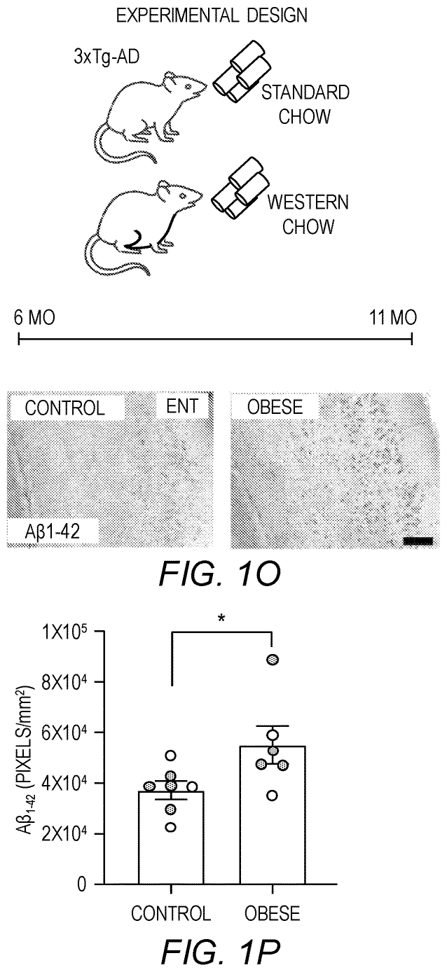

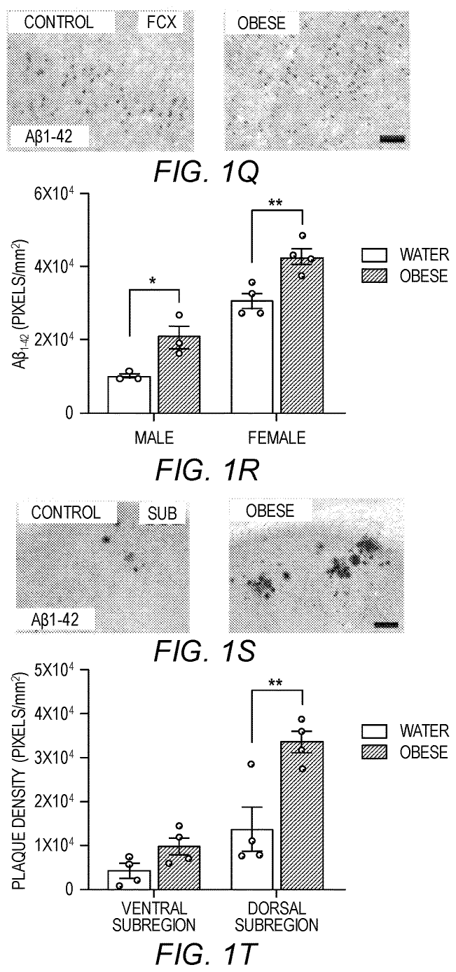

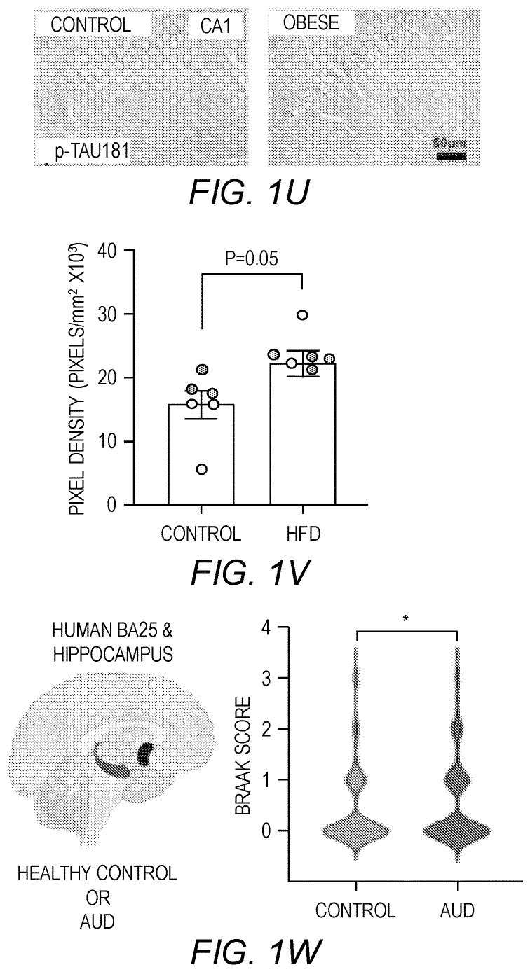

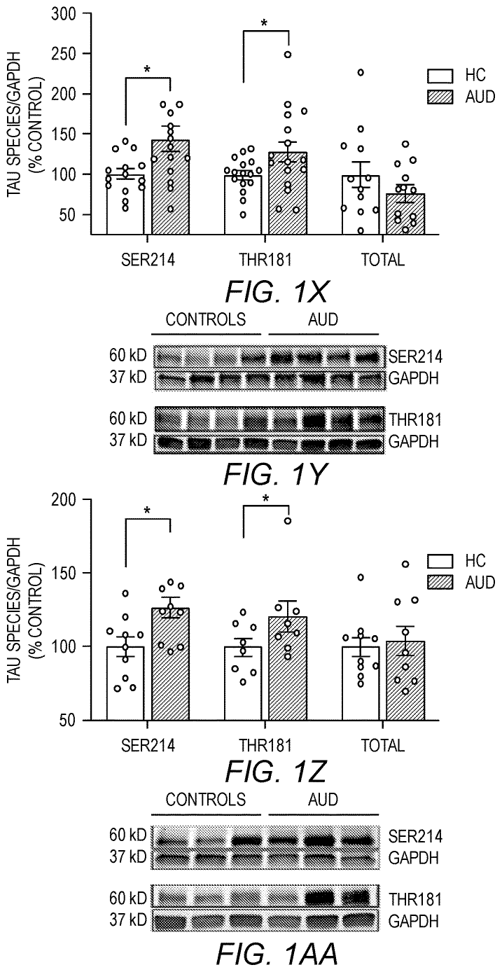

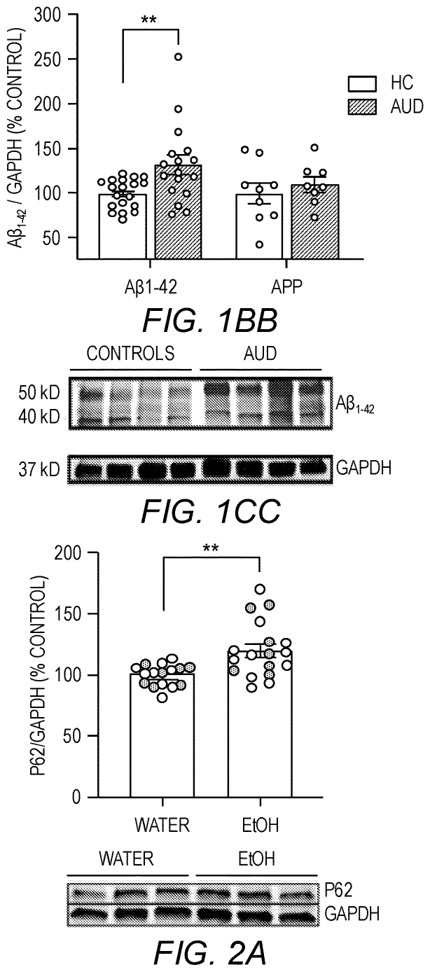

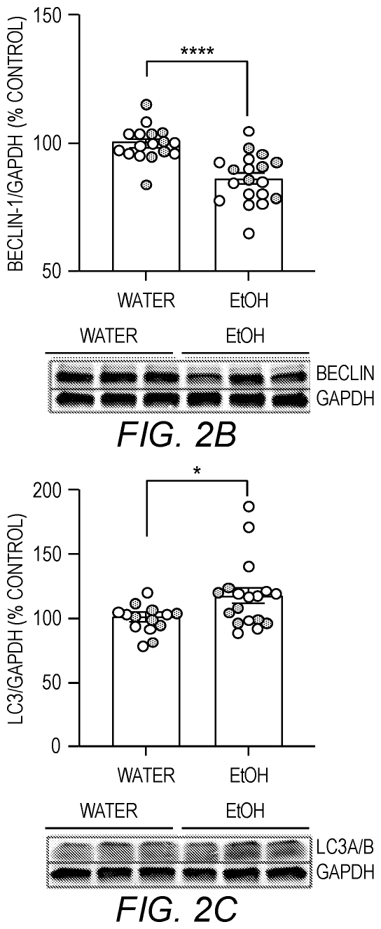

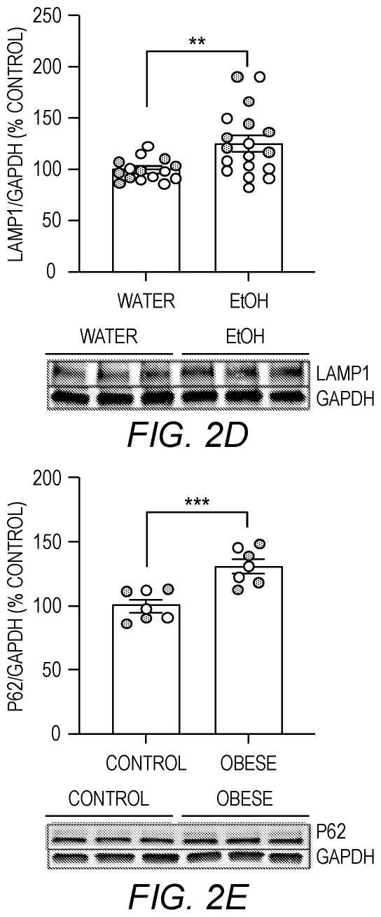

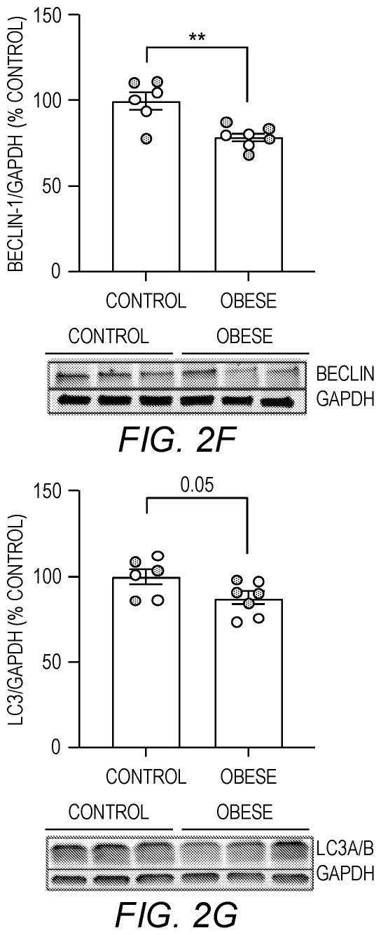

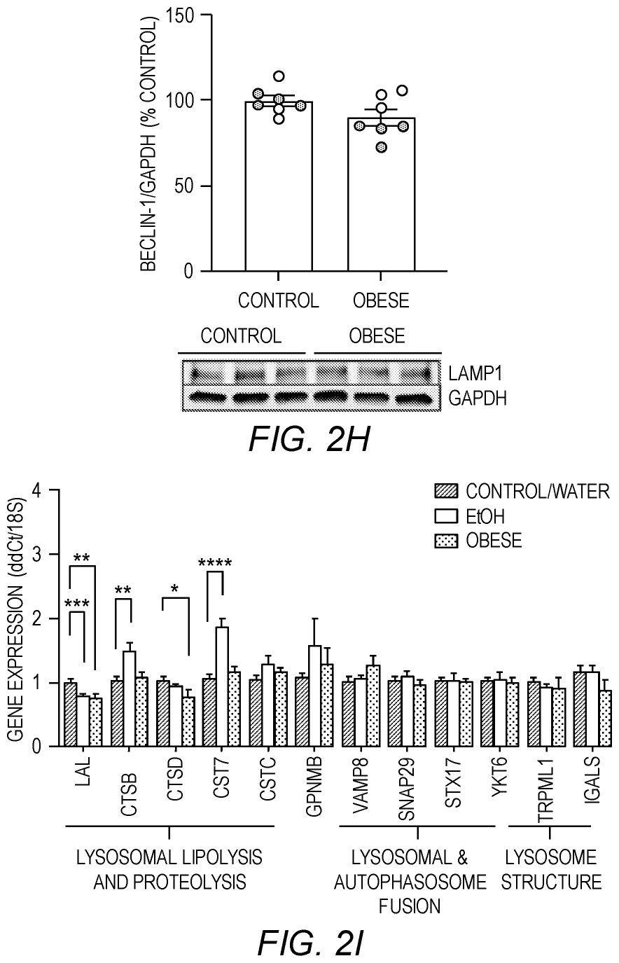

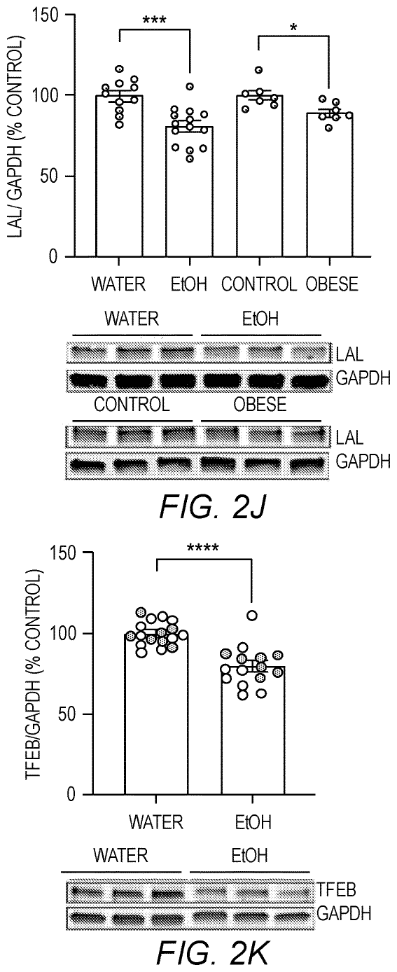

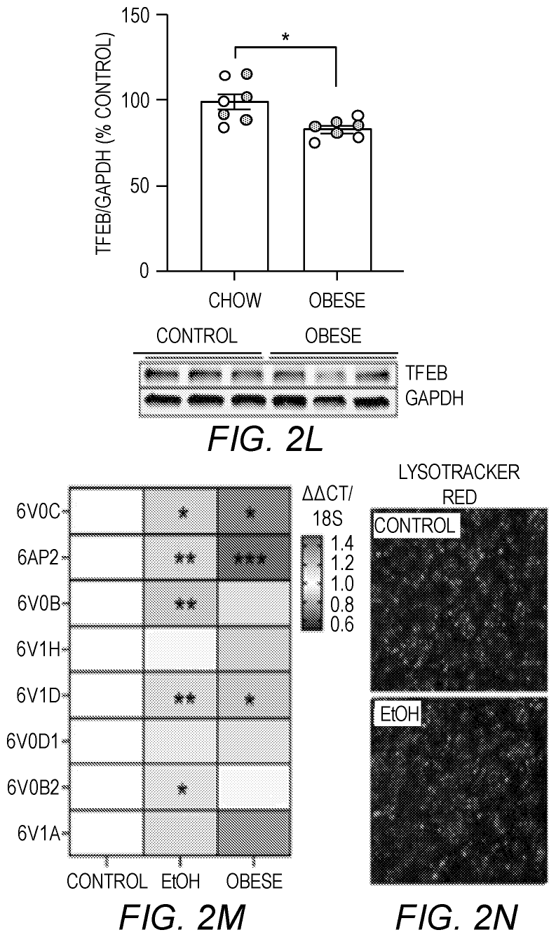

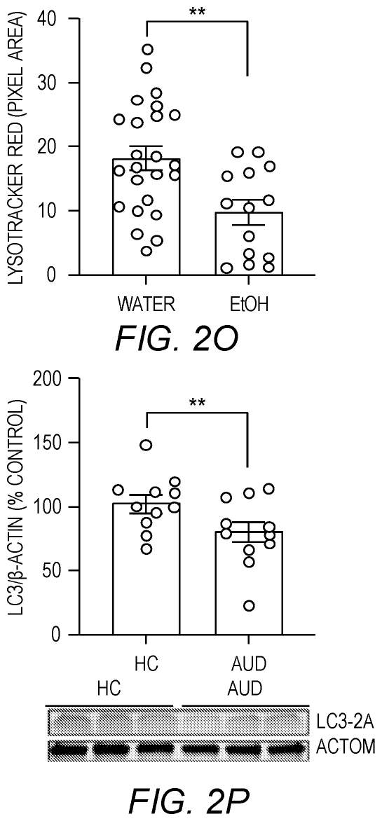

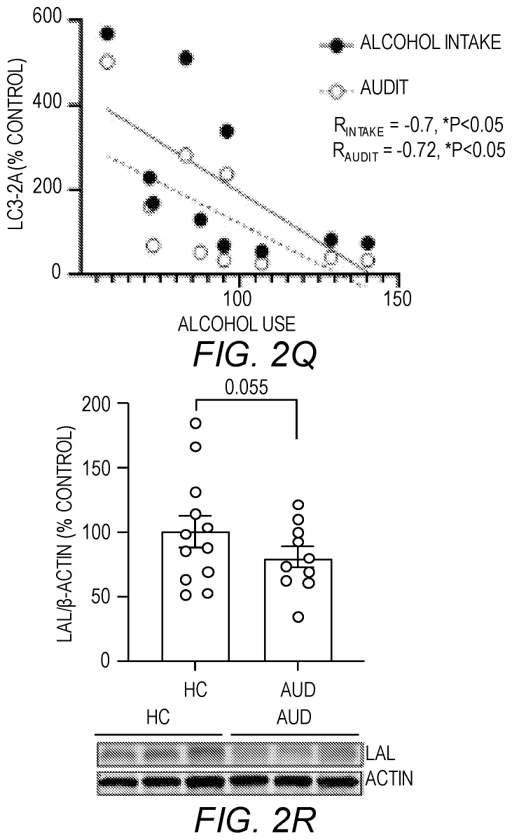

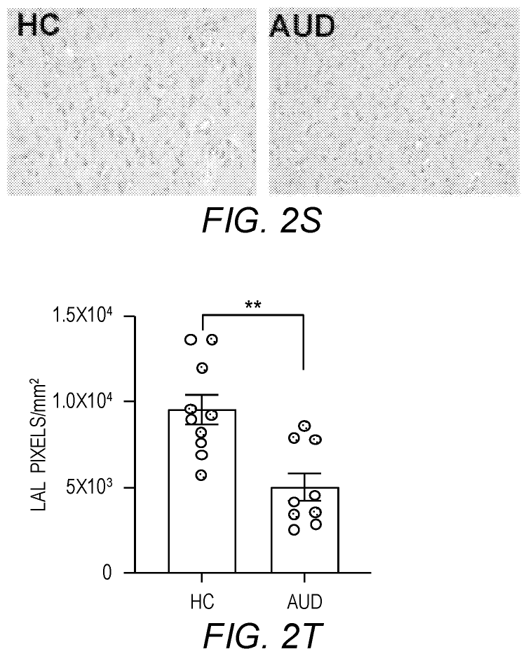

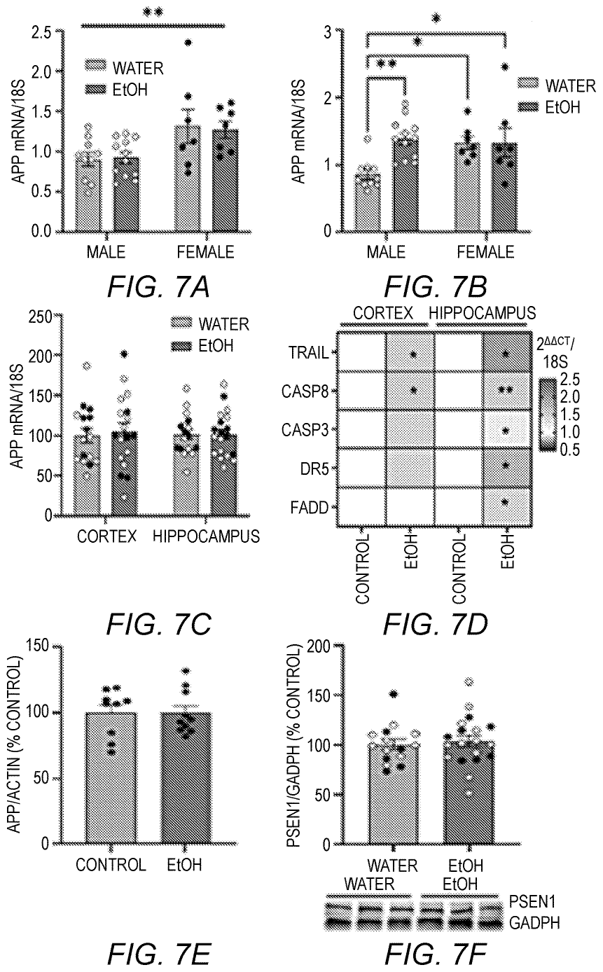

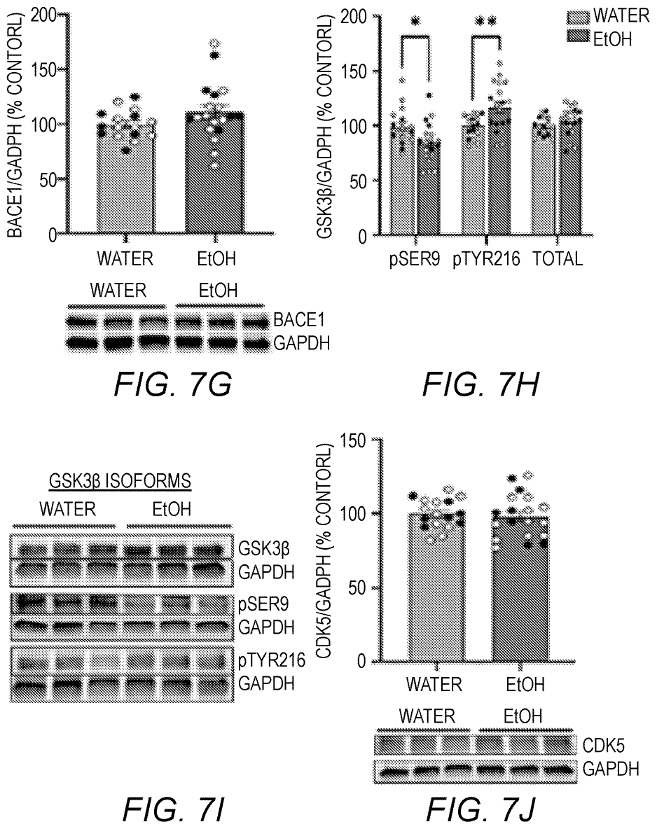

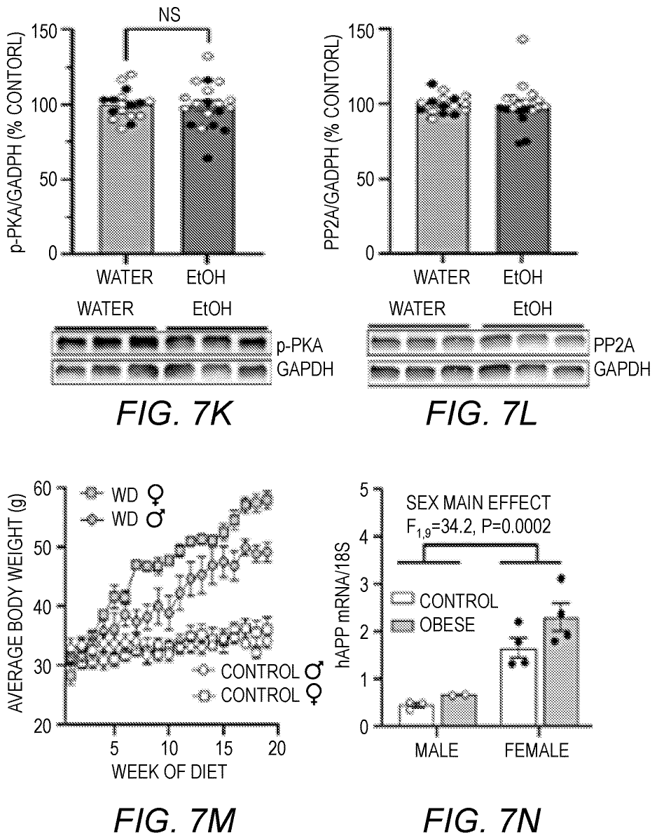

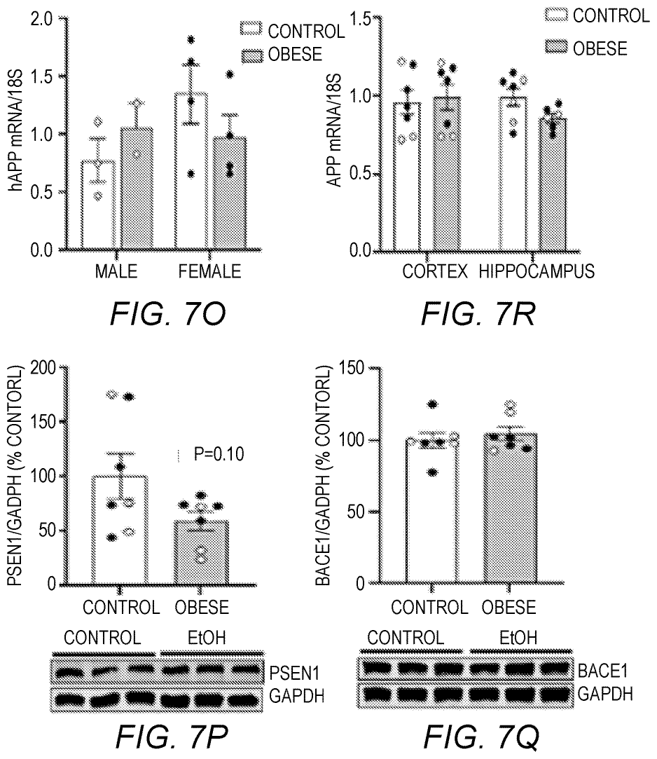

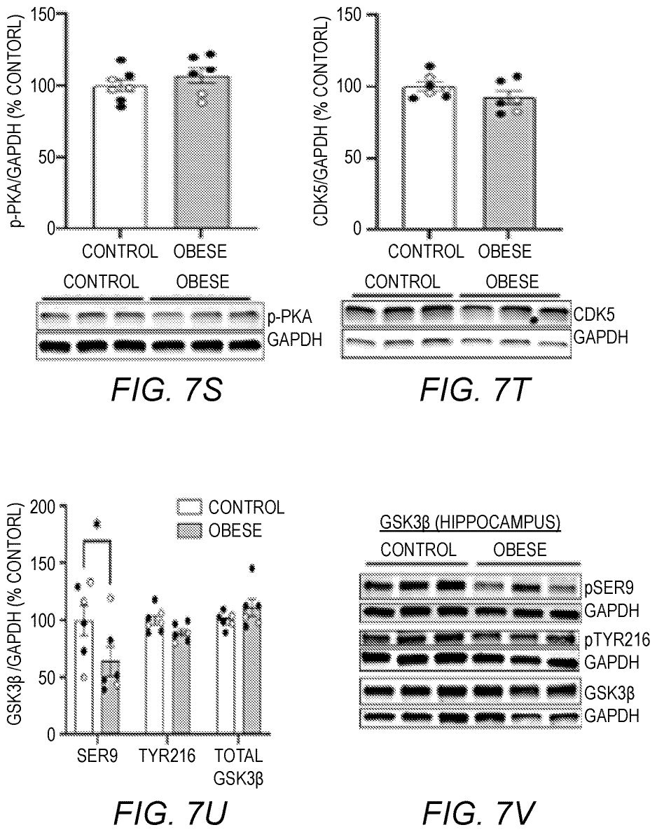

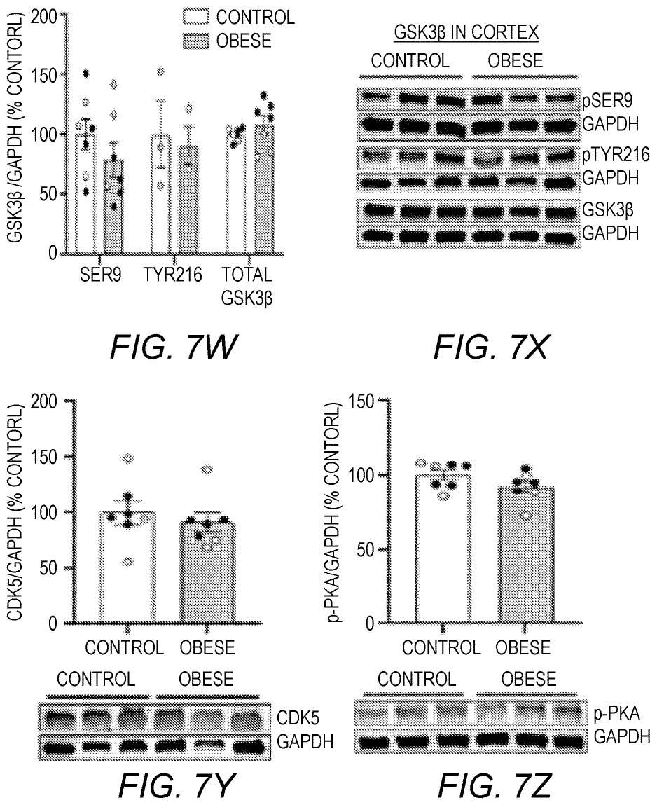

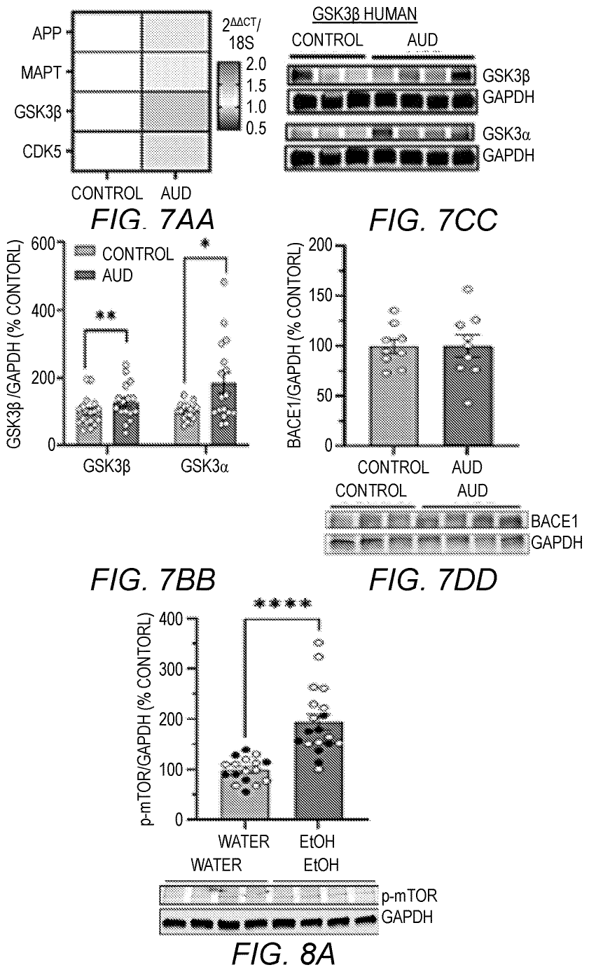

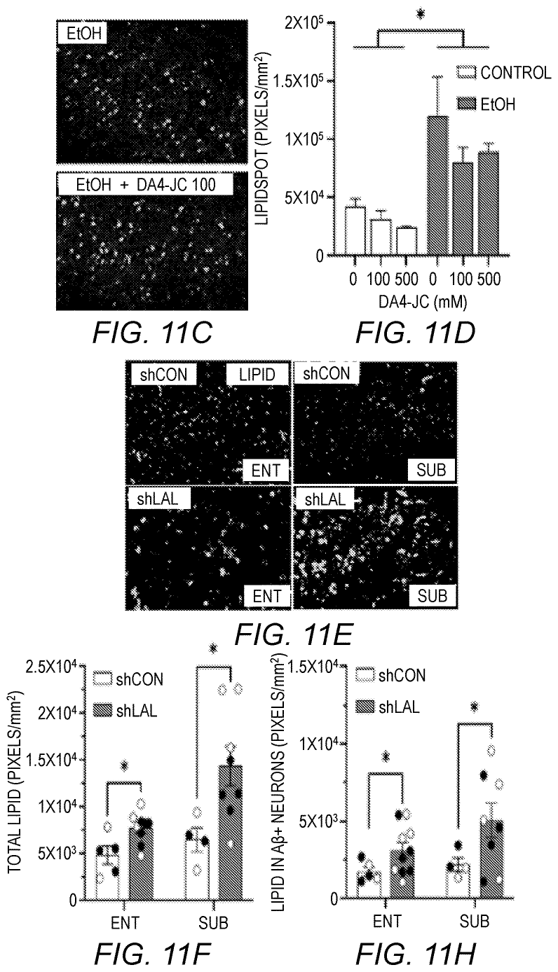

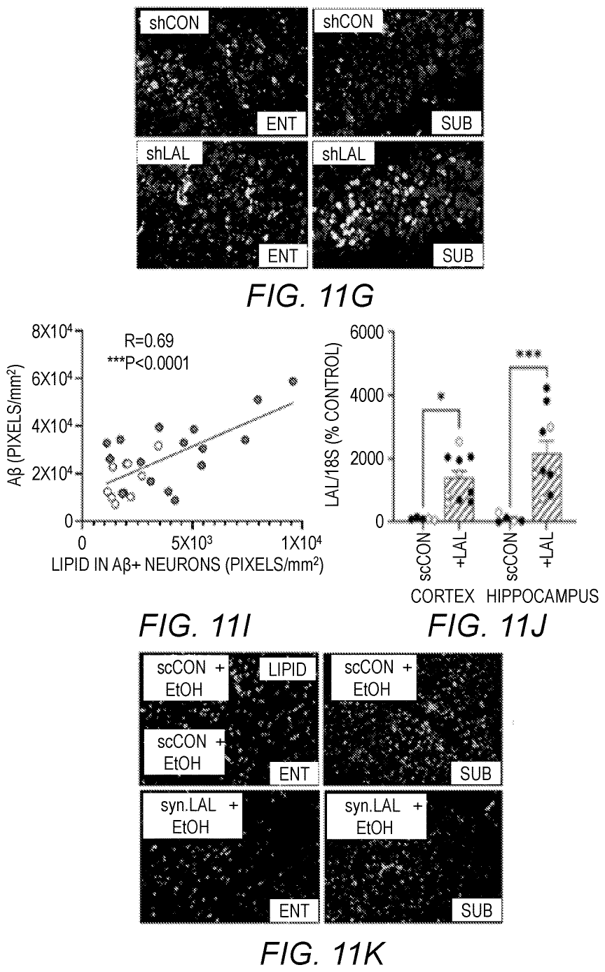

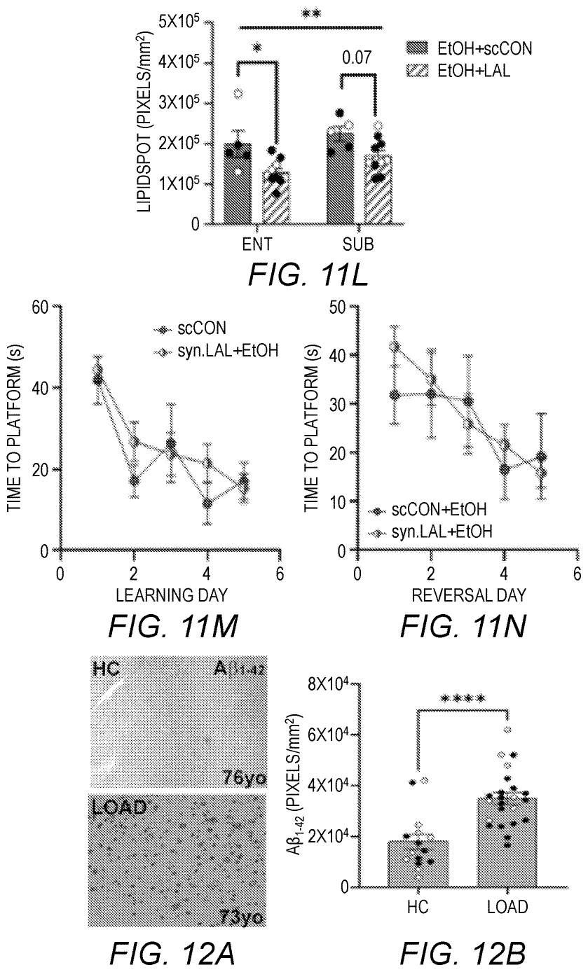

LOAD midlife risk factors promote pathology by disrupting neuronal Aβ metabolism. First, the impact of midlife heavy alcohol (i.e., ethanol) and diet-induced obesity was assessed on LOAD pathology in the entorhinal cortex (ENT), hippocampus, subiculum (SUB), and frontal cortex (FCX). The 3×Tg-AD mouse model (APPSwe, tauP301, Psen1^{tm1Mpm}) was used since it shows a progressive age-related increase in pathologic Aβ and tau species, seen first in entorhinal and hippocampal regions, followed by cortex at later ages, similar to LOAD²¹⁻²³ Mice were treated during midlife to match the LOAD epidemiology, when Aβ pathology is ~30-40% of peak levels in 3×Tg-AD. Since mice metabolize ethanol ~8-times more rapidly than humans²⁴; a dose was used that produces average blood alcohol concentrations during intoxication similar to those in humans (average: ~25 mg/dL, peak: ~290 mg/dL) 25. Ethanol increased pathogenic intraneuronal Aβ₁₋₄₂ by ~55-60% in the ENT, FCX and SUB in both sexes (FIG. 1A-1E). At this age males do not show amyloid plaques; however, females had amyloid plaques in the SUB that were increased 2-fold by ethanol (FIGS. 1E-1F). Ethanol also increased p-tau-181, a marker for LOAD in humans^{26,27}, by 40% in CA1 and FCX (FIGS. 11-1J). These increases occurred without any effect on expression of the human APP (hAPP) or MAPT transgenes in cortex (FIGS. 7A-7C). Increases in Aβ₁₋₄₂ and p-tau-181 were accompanied by neurodegeneration, with a 2.5-fold increase in the TUNEL apoptotic stain in the ENT (FIGS. 1K-1L) and induction of extrinsic apoptotic cell death genes implicated in AD (TRAIL, caspase-8, death receptor 5 and FADD, FIG. 7D). WT mice were further assessed to assess the impact of AD genetics, where ethanol likewise increased total Aβ in WT ENT by 36%, (FIGS. 1M-1N), without altering expression of mouse APP (FIG. 7E). Furthermore, in 3×Tg-AD, ethanol had no effect on levels of cortical presenilin 1 (PSEN1) or BACE1 protein (FIGS. 7F-7G) and had minimal impacts on the major tau-phosphorylating kinases (pSer9-GSK3β, pTyr216-GSK3β, p-PKA) and the tau phosphatase PP2A (FIGS. 2H-2L). Midlife likewise obesity increased Aβ₁₋₄₂ at 11 months 0.5 to 2.5-fold in ENT, FCX, and SUB (2.4-fold) (FIGS. 1O-1T). Similar to ethanol, this occurred without any differences in expression of the hAPP transgene (FIG. 7N-7O) or levels of PSEN1 or BACE1 (FIGS. 7P-7Q). Obese mice also had increased p-tau181 in CA1 (41%, p=0.05, FIGS. 1U-1V) with no changes in expression of the hMAPT transgene (FIG. 7R), nor tau phosphorylating enzymes GSK3B, p-PKA, CDK5, p16 (FIGS. 7S-7Z).

Findings in rodents were consistent with human postmortem ventromedial prefrontal cortex (vm-PFC/BA25) and hippocampus from individuals with alcohol use disorder (AUD) or moderate-drinking age-matched controls. Though none of these individuals had a co-morbid diagnosis of AD, AUD brains had higher Braak scores than controls (FIG. 1W) and increased levels of p-tau214 and p-tau181 in vm-PFC (FIGS. 1X-1Y, ~50% and 30%, respectively) and hippocampus (FIGS. 1Z-1AA, 20-27%) without increases in total tau. Aβ₁₋₄₂ was also increased in AUD vm-PFC (31%, FIG. 1BB-1CC), with no changes in levels of amyloid precursor protein (APP). Similar to findings in 3×Tg-AD, gene expression of APP, MAPT, GSK3 and CDK5 were not changed in AUD hippocampus (FIG. 7AA). Tau-phosphorylating isoforms of GSK3 were increased in AUD BA25 (FIG. 7BB-7CC), while levels of Aβ₁₋₄₂ producing BACE1 protein were unchanged (FIG. 7DD). Together, this supports that both midlife alcohol and obesity promote LOAD pathology without altering Aβ or tau gene expression or levels of Aβ modifying enzymes.

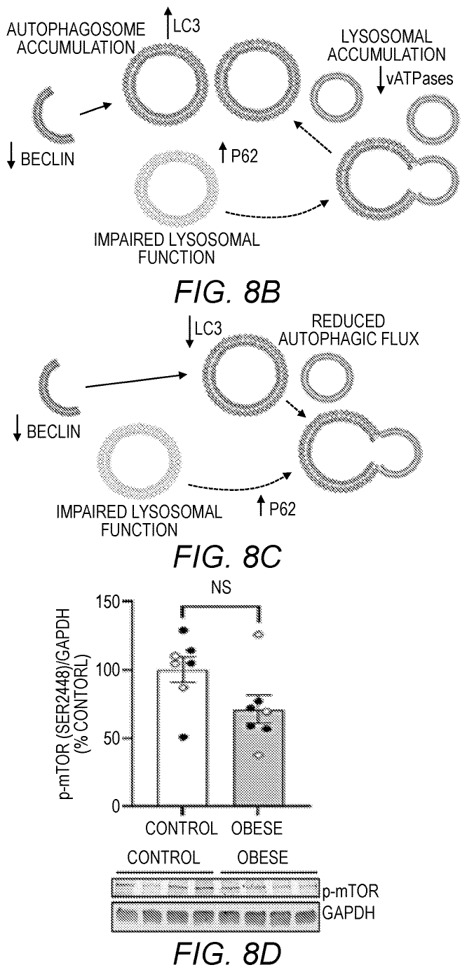

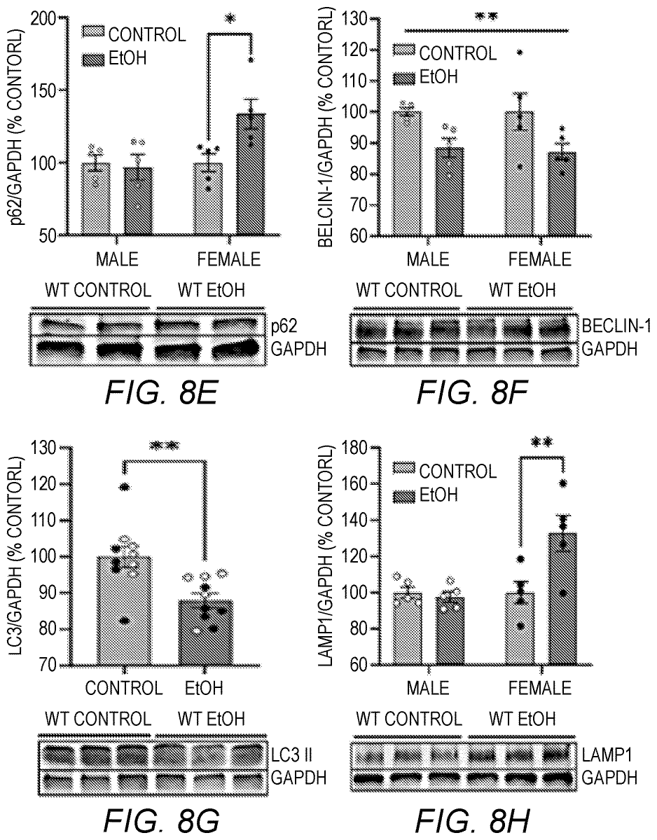

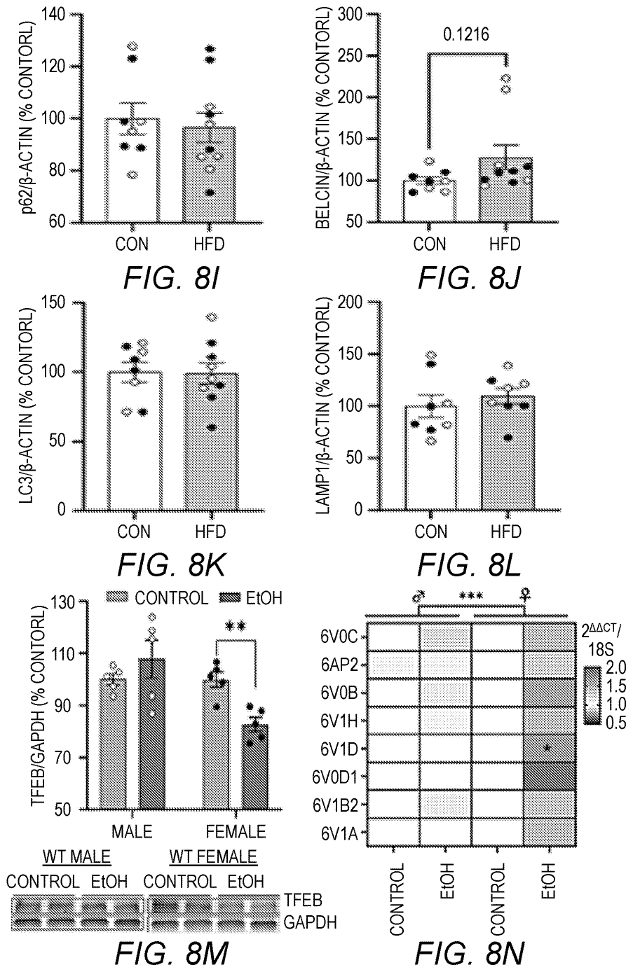

Disruption of autophagy and lysosomal function by heavy alcohol and obesity during midlife. Since neurochemical findings suggested midlife LOAD risk exposures disrupt neuronal Aβ metabolism, the autophago-lysosomal system, the main site of intraneuronal Aβ degradation was assessed. Midlife ethanol increased p62 (20%), reduced the autophagy initiator Beclin (20%), increased the autophagosome elongation factor LC3-II (20%), and increased the mature lysosome protein LAMP1 (26%) and increased phosphorylated mTOR (p-mTOR, Ser4228) (FIG. 8A, 2-fold) in 3×Tg-AD cortex. In WT mice ethanol increased P62 in females, reduced Beclin in both sexes, and reduced LC3 in both sexes, with LAMP1 accumulation in females (FIGS. 8E-8H). These findings are consistent with a loss of autophagic flux secondary to lysosomal dysfunction and a secondary reduction in autophagy initiation (FIG. 8B). Diet-induced obesity also disrupted autophagic flux, though differently than ethanol (FIG. 8C). Obesity increased p62 (33%), reduced Beclin (21%), and reduced LC3 (12.5%), total without changing LAMP1 (FIG. 2E-2H) or p-mTOR Ser4228 (FIG. 8D). In 11-month obese WT mice, no significant changes in P62, Beclin, LC3 or LAMP1 were seen (FIGS. 8I-8L).

To further assess lysosomal function, the expression of lysosomal lipases and proteases, regulators of lysosome/autophagosome fusion, and lysosomal structural genes were measured (FIG. 2I). Neither ethanol nor obesity significantly altered the expression of facilitators of lysosome/autophagosomes fusion (VAMP8, Snap29, Stx17, YKT6) nor regulators of lysosomal structure (TRPML1, lgals). However, both LOAD risk exposures reduced expression of the lysosomal acid lipase gene (LIPA, 20-25%) and protein (10-20%, FIG. 2J). This occurred with reductions in TFEB, a regulator of lysosomal gene expression³⁰ (FIGS. 2K-2L) and reduced lysosome acidifying v-ATPases (FIG. 2M). Similar reductions in TFEB and lysosomal v-ATPases were seen in WT female mice with ethanol (FIGS. 8M-8N). Of note, TFEB was expressed robustly in cortical neurons (FIG. 8O). Ethanol reduced lysosomal acidification in the ENT of hippocampal-entorhinal brain slice cultures was also found (HEBSC, FIGS. 2N-2O). In human AUD brain, LC3 was reduced 20% and correlated negatively with lifetime alcohol use and AUDIT score (FIG. 2Q). LAL protein levels were also reduced in AUD vm-PFC (FIG. 2R, 21% by WB in Cohort A, p=0.055, FIGS. 2S-2T, 47% IHC, cohort 2, **p<0.01). Other AD-associated regions also had reduced LAL in AUD including ENT (39%), CA1 (27%), and SUB (54%) (FIGS. 8P-8Q). Together this indicates these two common midlife LOAD risk factors disrupt autophago-lysosomal flux and reduce LAL in rodents and humans.

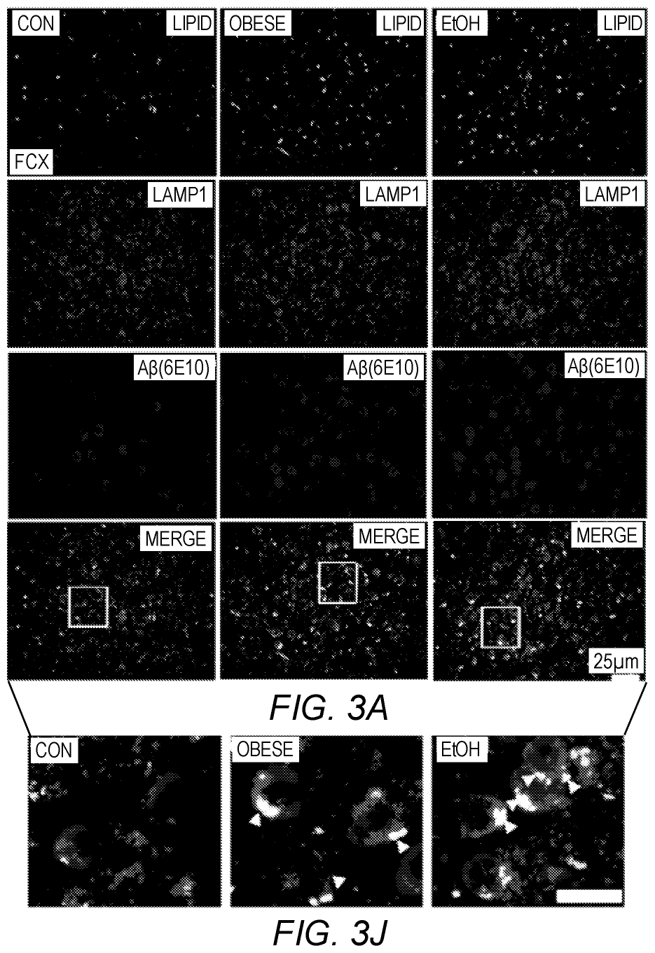

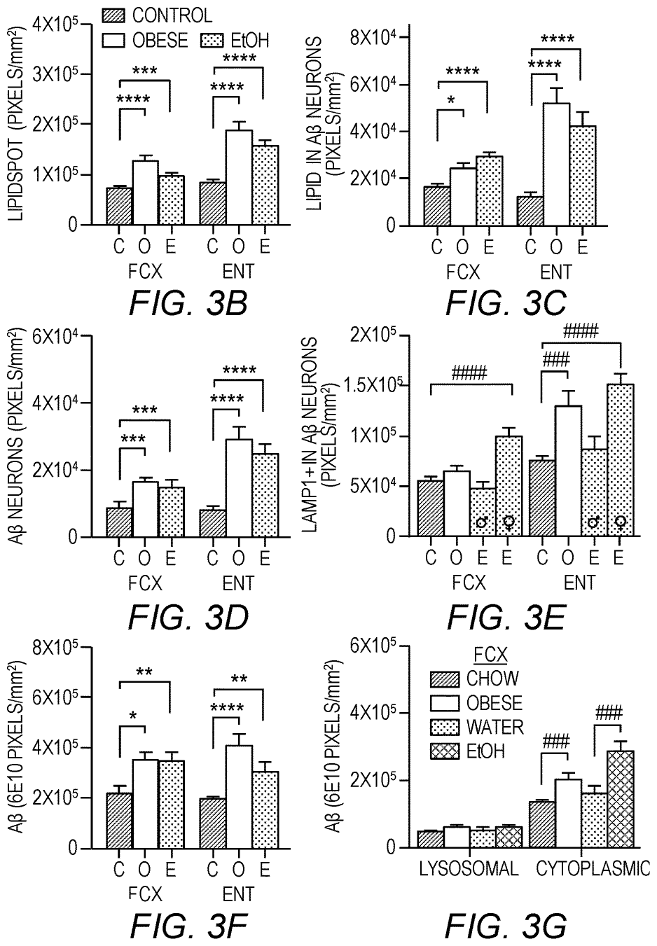

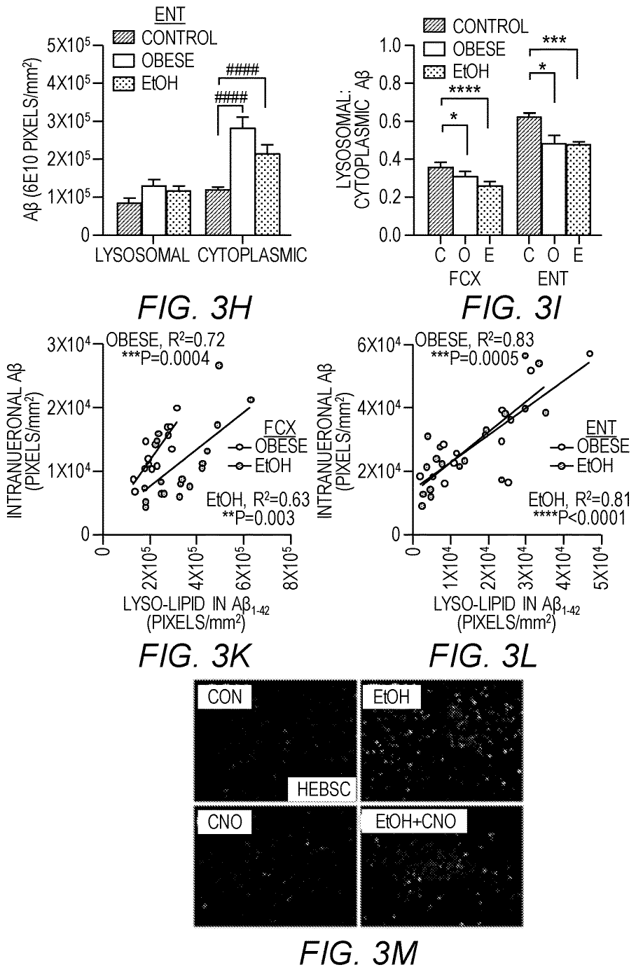

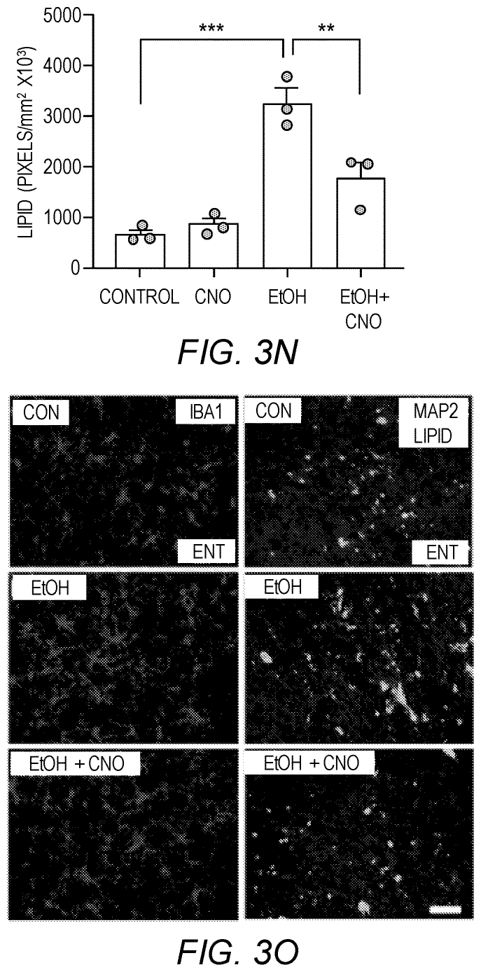

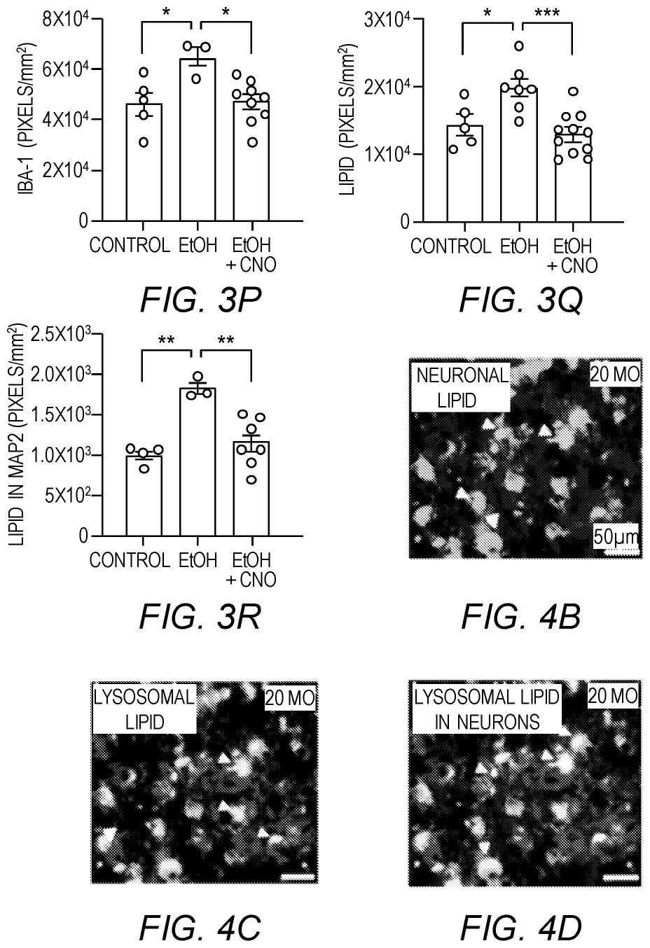

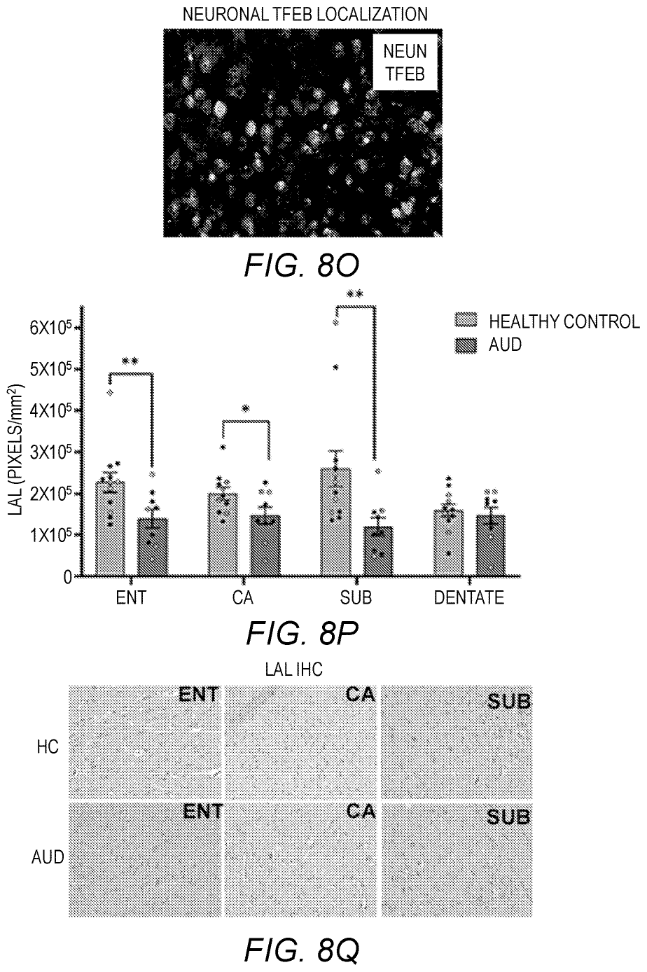

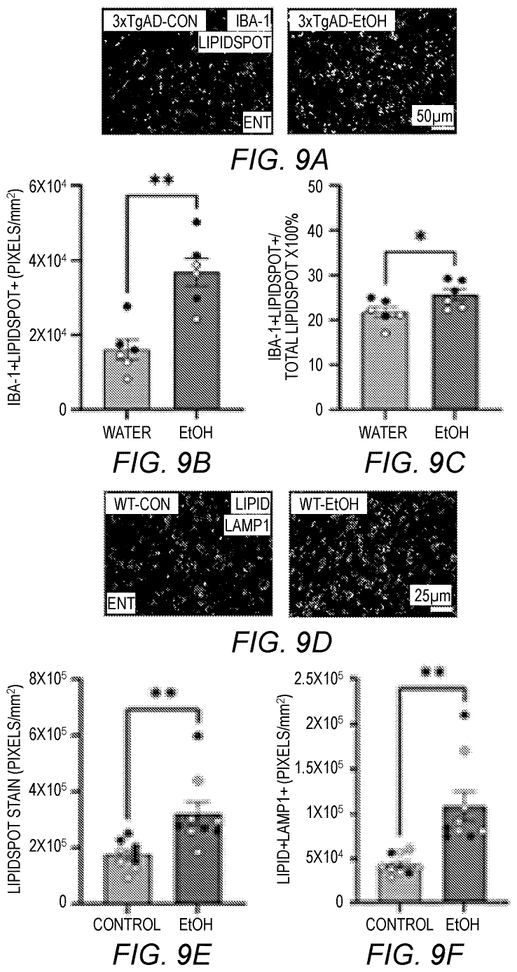

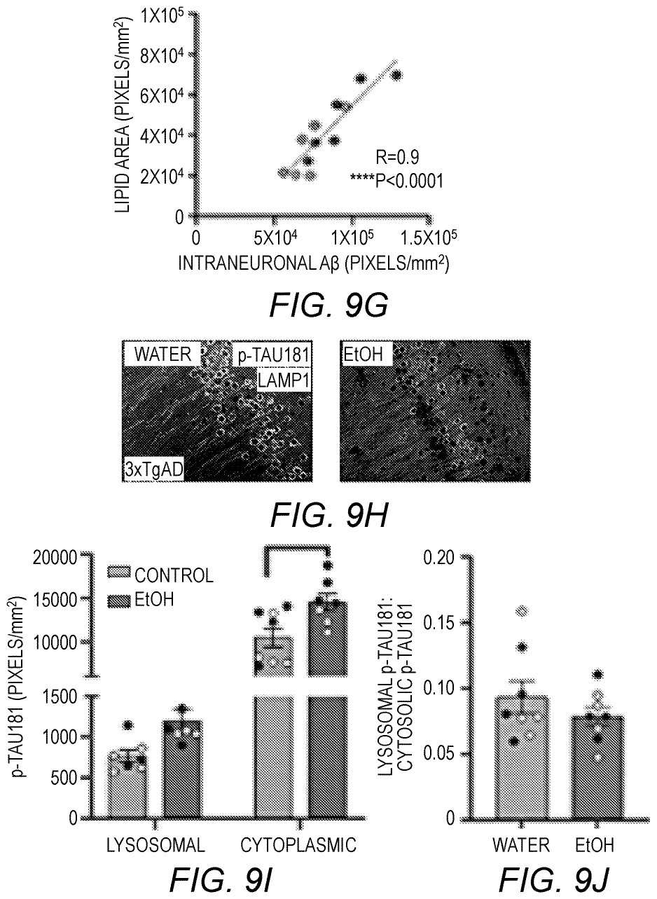

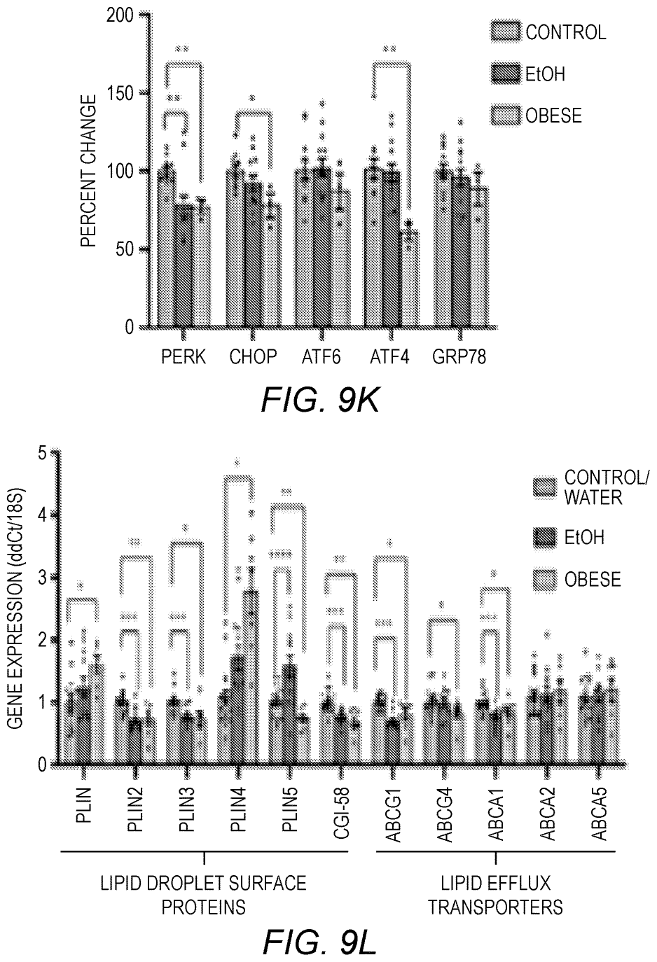

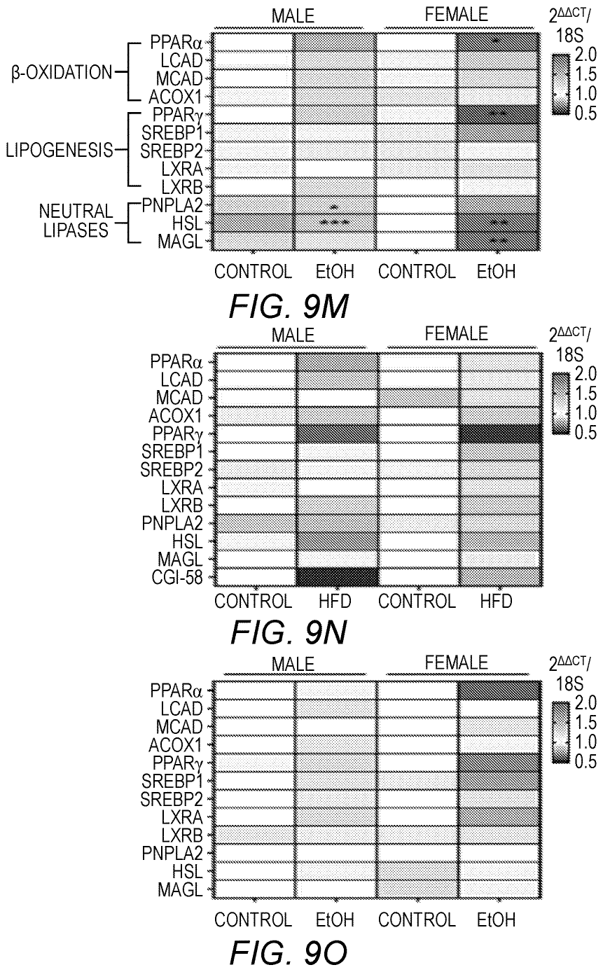

Neuronal lysosomal lipid (NLL) accumulation occurs with early Aβ pathology and aging. Given the intersection of autophagic and lipophagic machinery with the loss of LAL, determining whether NLL promotes accumulation of Aβ was investigated. Total cytosolic lipid was increased with both LOAD risk factors (FIGS. 3A-3B). In FCX, lipid was increased 80% with obesity and 38% by ethanol (FIG. 3B, treatment effect F_{2,40}=20.07, p<0.0001). Greater increases were seen in the ENT, which develops AD pathology prior to the FCX, with a 120% increase in obese mice and an 88% increase with ethanol (FIG. 3B, F_{2,41}=35.84, p<0.0001). Significant increases in lipid levels in Aβ-containing neurons were found in the FCX (FIG. 3C, F_{2,39}=15.72, p<0.0001, obesity: 46%, ethanol: 75%) and the ENT (F_{2,29}=23.45, p<0.0001, obesity: 2.8-fold, ethanol: 2.2-fold). Increases in cytosolic lipid were also found in microglia, with ethanol increasing the level of microglial lipid by 2-fold (FIGS. 9A-9B) with ~25% of total lipid localized to microglia (FIG. 9C) and 25% within Aβ+ neurons. Both obesity and ethanol each increased lysosomal lipid in Aβ+ neurons by ~75% in FCX (FIG. 3D, F_{2,41}=15.73, p=0.0001), while in the ENT obese mice showed a 3.8-fold increase and ethanol-treated mice a 3.3-fold increase with obesity (F_{2,40}=32.1, p<0.0001). The accumulation of lysosomal lipid in neurons of 3×Tg-AD mice was associated with an increase in lysosome number. LAMP1+lysosomes in Aβ-containing neurons were increased in the FCX of ethanol-treated females (~2-fold, F_{3,32}=16.46, p<0.0001, ###p<0.0001, Sidak's post-test) as well as in the ENT (F_{3,43}=16.89, p<0.0001), suggesting the accumulation of lysosomal lipid may promote their dysfunction and accumulation (FIG. 3E).

Consistent with FIGS. 1A-1CC, both ethanol and obesity increased Aβ by 52% and 56%, respectively, in FCX (FIG. 3F, F_{2,41}=7.5, p=0.002) and by 2.1-fold (obesity) and 59% (ethanol) in the ENT (F_{2,41}=15.3, p=0.0001). These increases in Aβ were primarily in the extra-lysosomal cytoplasm with ethanol (F_{1,59}=14.38, p=0.0004, 2-way ANOVA treatment effect) and obesity (F_{1,20}=19.52, p=0.0003) in FCX (FIG. 3G) and ENT (FIG. 3H, F_{2,82}=20.38, p=0.00001). Subsequently, the Aβ lysosomal:extra-lysosomal cytoplasmic (lyso:cyto) ratios were reduced by ~20% in FCX (FIG. 3I, F_{2,41}=14.50, p<0.0001) and ENT (FIG. 3I, F_{2,40}=8.788, p=0.0007). With each risk factor, the level of intraneuronal Aβ was strongly correlated with the level of lysosomal lipid in Aβ neurons. In FCX, both obesity (R²=0.72, ***p=0.0004) and ethanol (R²=0.63, **p=0.003) showed strong positive correlations (FIG. 3K), as did ENT (FIG. 3L, obese: R²=0.83, ***p=0.0005, ethanol: R₂-0.81, ****p<0.0001). WT mice treated with ethanol similarly had increased lipid levels (83%, FIGS. 9D-9E) as well as increased lysosomal lipid levels (2.5-fold) in ENT after chronic ethanol (FIG. 9F). A strong positive correlation was found between total Aβ and total lipid in WT mice as well (FIG. 9G, R=0.9, ****p<0.0001), indicating AD transgenes are not required for this effect. Assessment of p-tau in CA1 of hippocampus found increases in both lysosomal and cytoplasmic levels with ethanol (FIGS. 9H-9I), and no significant change the lyso:cyto ratio (FIG. 9J). Proinflammatory microglial activation has been found to promote intraneuronal Aβ accumulation^{20,31}. Therefore, it was assessed if proinflammatory microglia promote lipid accumulation. HEBSCs from 3×Tg-AD mice were transfected with a Gi inhibitory DREADD (AAV9.CD68.hM4di) to inhibit proinflammatory signaling caused by ethanol as previously reported^{32,33} and were then treated with ethanol +/− the DREADD ligand CNO. Ethanol-induced increases in cytosolic lipid were prevented by microglial inhibition (FIGS. 3M-3N). This was confirmed in vivo using CX3CR1Cre^{ERT}.hM4di mice that received ten days of ethanol+/−CNO. Ethanol caused proinflammatory activation of microglia in ENT seen by IF that was prevented by the microglial Gi DREADD ligand CNO (FIGS. 30-3P). Ethanol-induced increases in total and neuronal lipid were prevented by microglial inhibition (FIGS. 3Q-3R). Together, this suggested a direct relationship between lysosomal lipid levels and intraneuronal Aβ pathology, with proinflammatory microglia promoting lipid accumulation within neurons.

Factors that promote intracellular lipid accumulation such as endoplasmic reticulum (ER) stress and lipid droplet coating proteins were assessed. Increases in expression of key genes associated with the ER stress response were not found in 3×Tg-AD cortex (FIG. 9K). Neither were robust changes found in lipid metabolism genes or cytosolic lipases with ethanol, obesity, or aging that could explain the enhanced lysosomal lipid profile in both sexes (FIGS. 9L-9O). However, there were alterations in the expression of lipid-droplet coating perilipins (PLINs) and lipid efflux transporters. Both ethanol and obesity reduced expression of PLIN2 and PLIN3, while ethanol also increased PLIN5 and obesity increased PLIN and PLIN4 (FIG. 9L). This suggests these risk factors are metabolic stressors which alter lipid droplet coating proteins in a manner that could reduce lysosomal degradation or cause lysosomal stress. Further, ethanol and obesity reduced expression of lipid efflux transporters ABCG1 and ABCA1 which could also promote elevations in cytosolic lipids. However, neither ethanol nor obesity caused convergent changes in gene expression of common regulators of lipid metabolism or production across both sexes (FIGS. 9M-9O). Therefore, the next focus was determining if loss of LAL causes NLL and Aβ accumulation.

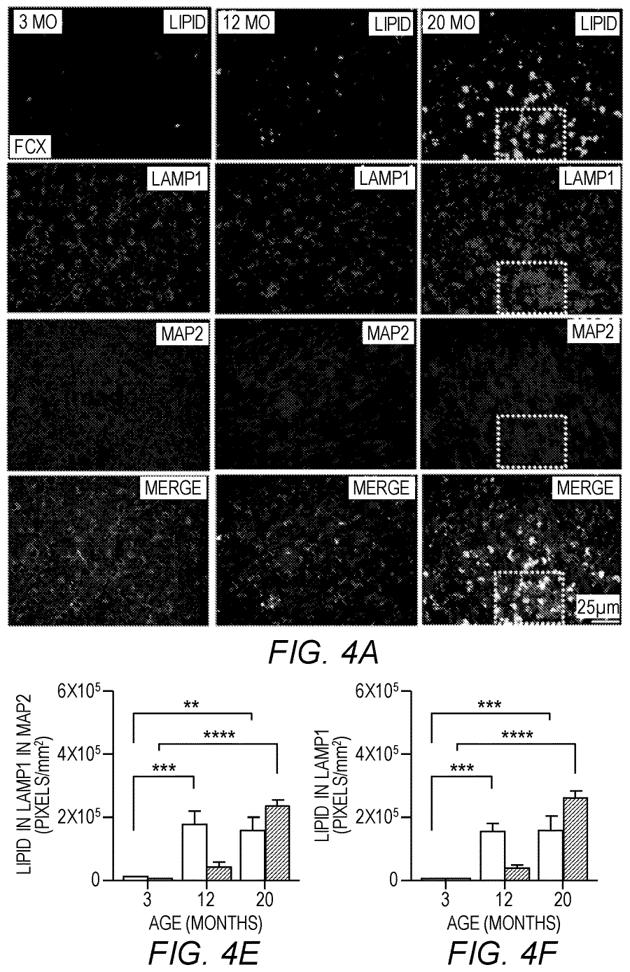

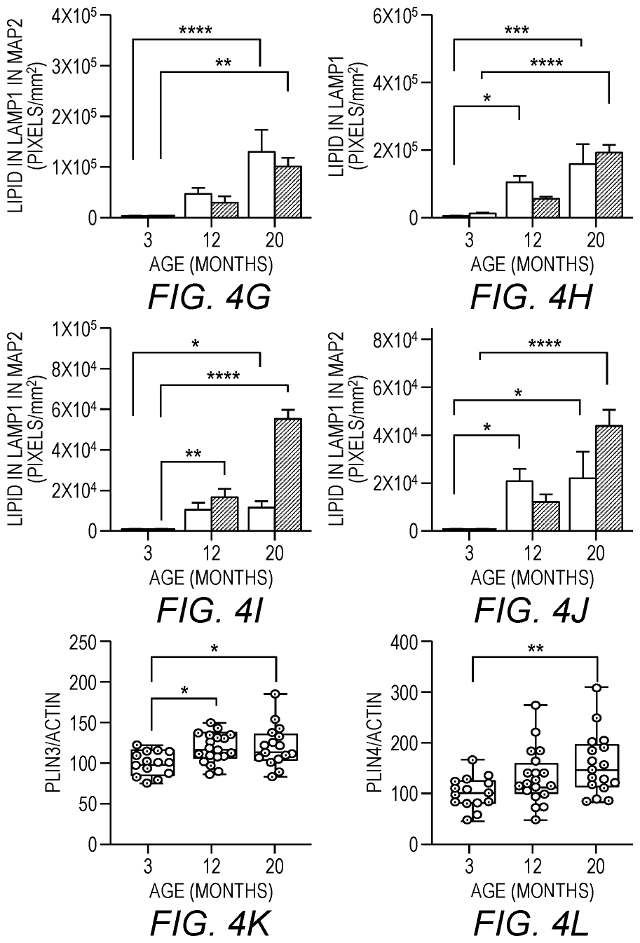

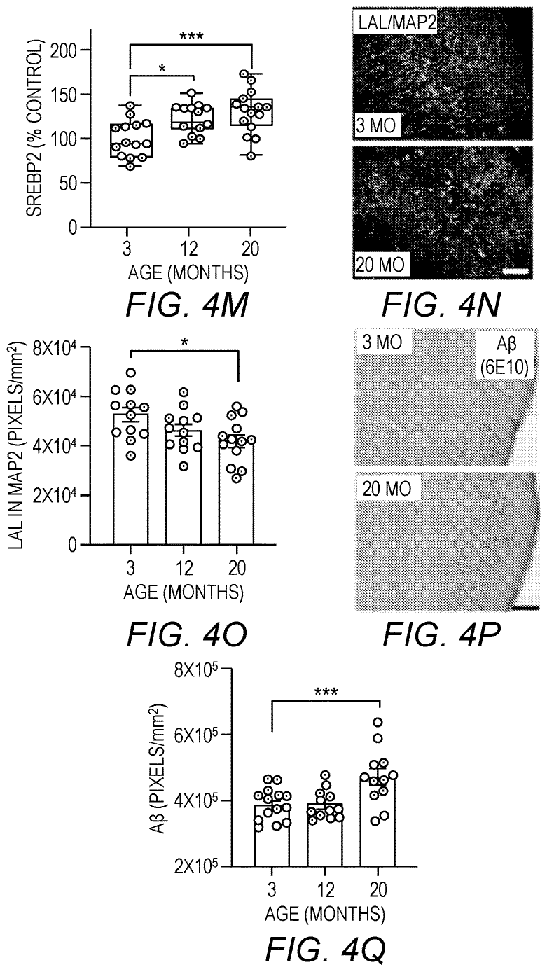

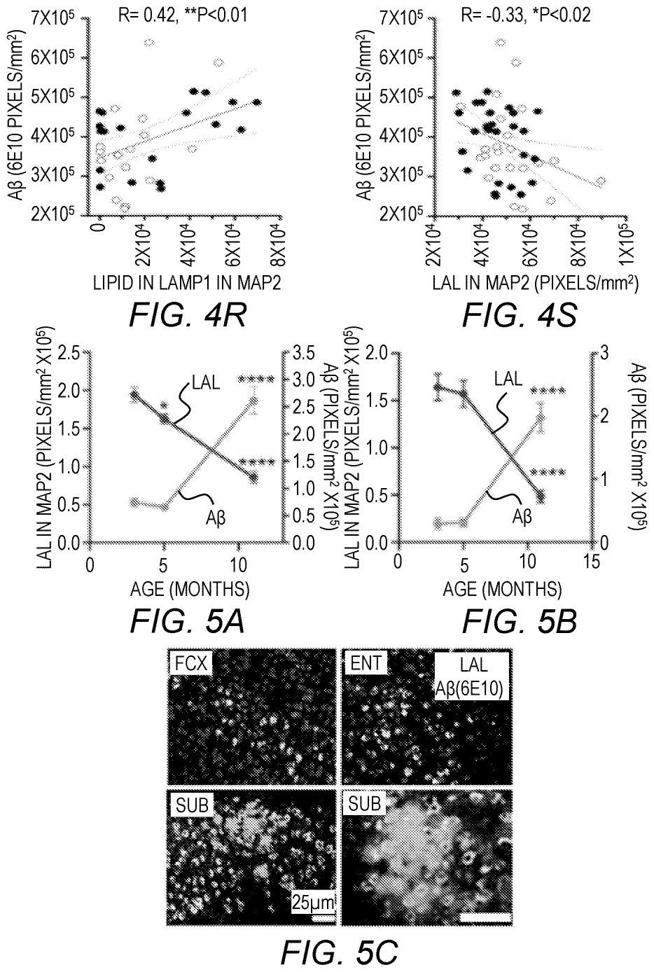

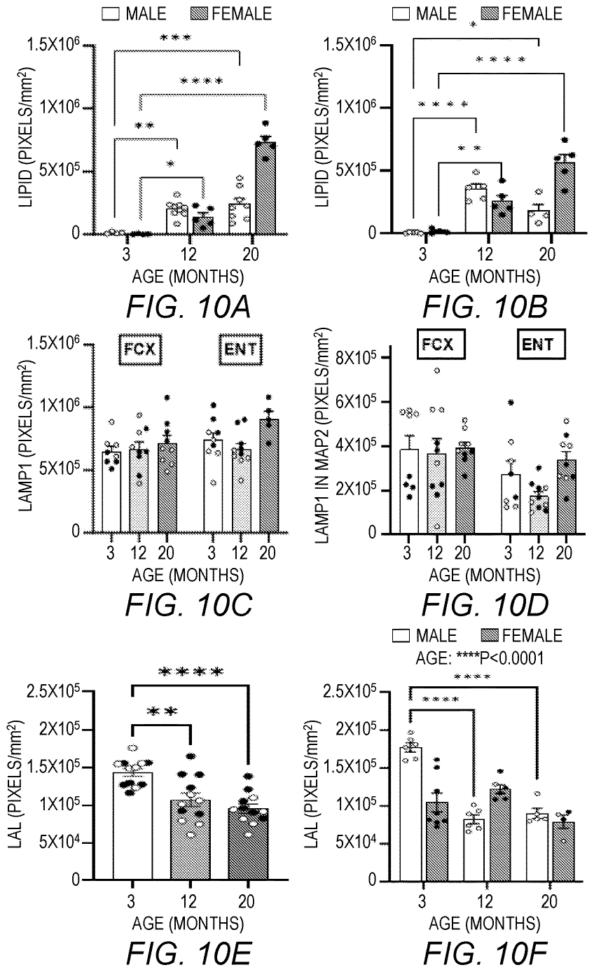

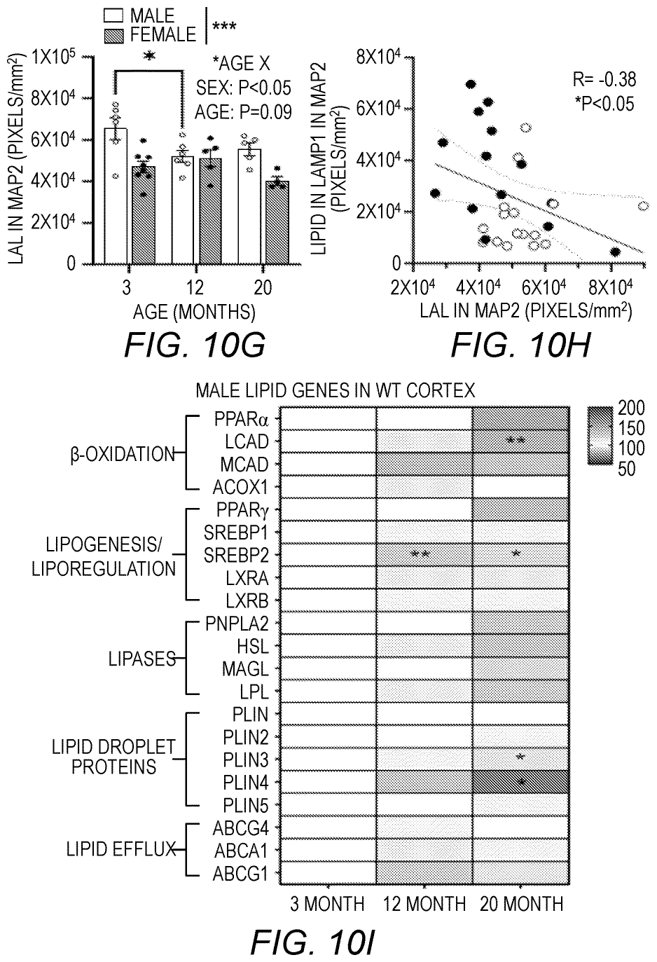

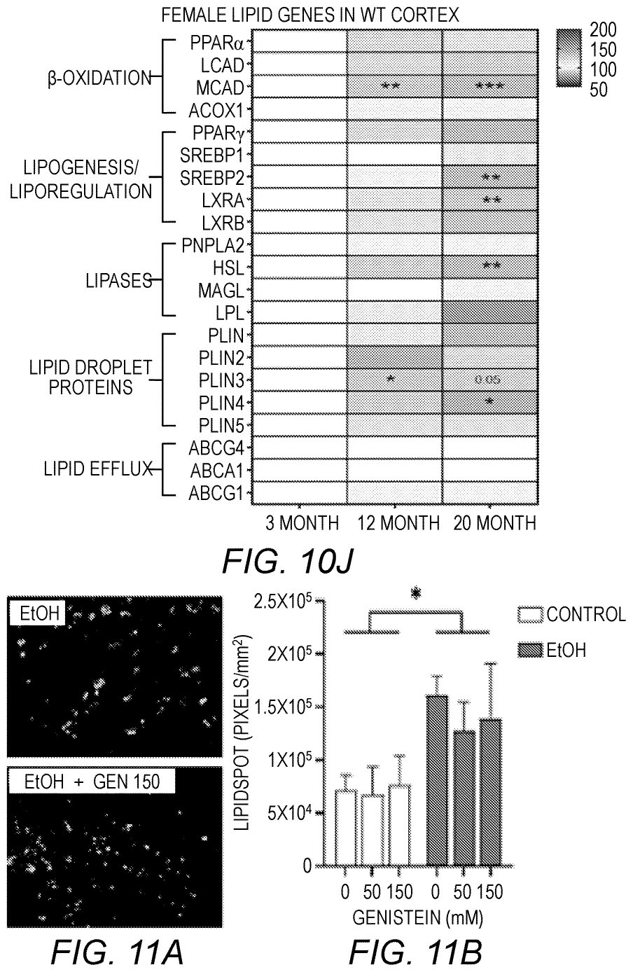

LAL is lost with aging and promotes NLL accumulation to drive Aβ accumulation. Aging is essential for the emergence of AD pathology in humans and 3×Tg-AD mice, suggesting underlying resilience mechanisms are lost with age. Therefore, to determine if accumulation of lipid within neuronal lysosomes is an underlying age-related risk factor for AD, WT mice at 3, 10 and 20 months of age were assessed. At 3 months, very little cytosolic lipid, neuronal lipid, lysosomal lipid, or NLL were seen (FIG. 4A). However, by 20 months there were profound increases in neurons and lysosomes (FIGS. 4B-4D) with some sex differences. In FCX, cytosolic lipid increased 280-fold from 3 to 20 months in females, with males reaching their 19-fold increase by 12 months and leveling out (FIG. 10A). Neuronal and lysosomal lipid also increased greatly in male and female FCX, with male levels stabilizing at 12 months (FIGS. 4E-4F). Similar magnitude changes were seen in ENT, with increases in total lipid (FIG. 10B), neuronal lipid, and lysosomal lipid (FIGS. 4G-4H) with age. No differences were seen in total LAMP1 or neuronal LAMP1 levels (FIGS. 10C-10D). However, robust increases in NLL were seen in FCX and ENT, that reached higher levels in females (FIGS. 4I-4J). Notably, expression of lipid-droplet coating proteins PLIN3 and PLIN4 increased in both sexes (FIGS. 4K-4L) as did the lipogenic factor Srebp2 (FIG. 4M). In general, females had a more active lipid regulatory profile than males (FIGS. 10I-10J). This includes key genes such as LXRα and metabolic genes LCAD and MCAD (FIGS. 10I-10J). In ENT, which is impacted early in AD, total LAL and neuronal LAL declined ~20-30% with age (FIGS. 4N-4O and FIG. 10E). In FCX, a main effect of age on total LAL was found with females having lower levels at 3 months than males (FIG. 10F, F_{2,29}=20.64, p<0.001). Age-related reductions in neuronal LAL in FCX were less robust than in ENT, though males showed a slight decline from 3 to 12 months (FIG. 10G). At 12 and 20 months in ENT and FCX, NLL was negatively correlated with neuronal LAL (FIG. 10H, R=−0.38, *p<0.05). Subsequently, total Aβ increased in WT ENT with age from 3 to 20 months (25%, FIGS. 4P-4Q), appearing to lag slightly behind LAL, was positively correlated with NLL (FIG. 4R, R=0.45, **p<0.01), and negatively correlated with neuronal LAL (FIG. 4S, R=−0.33, *p<0.02). Therefore, with aging in WT and risk exposure in AD mice, NLL increased and was strongly associated with Aβ accumulation.

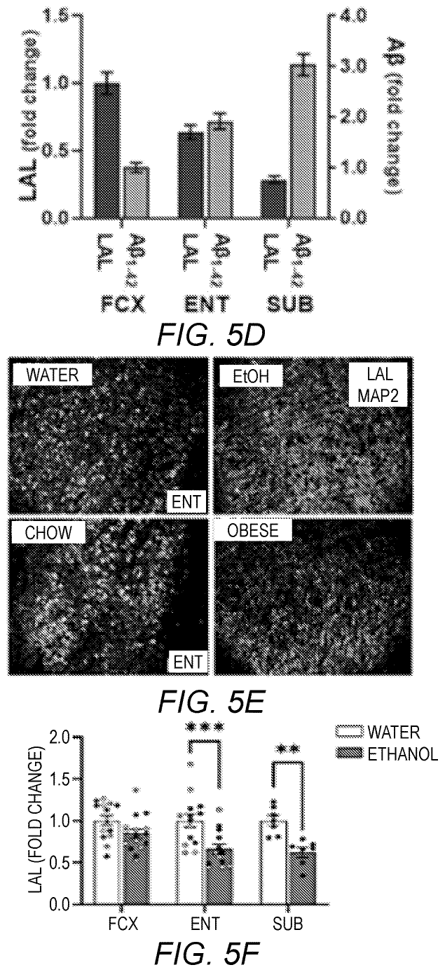

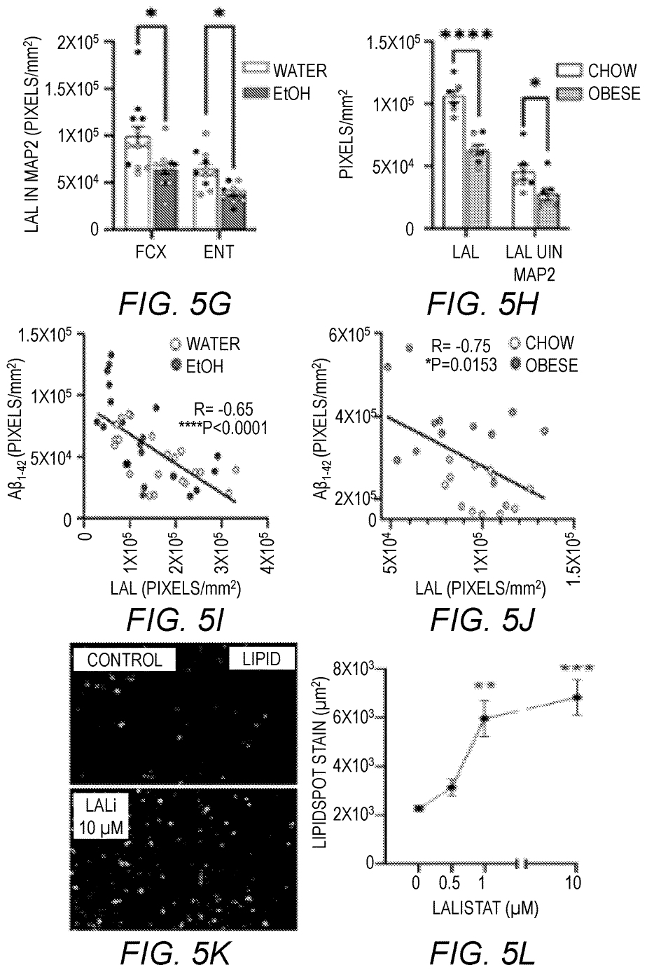

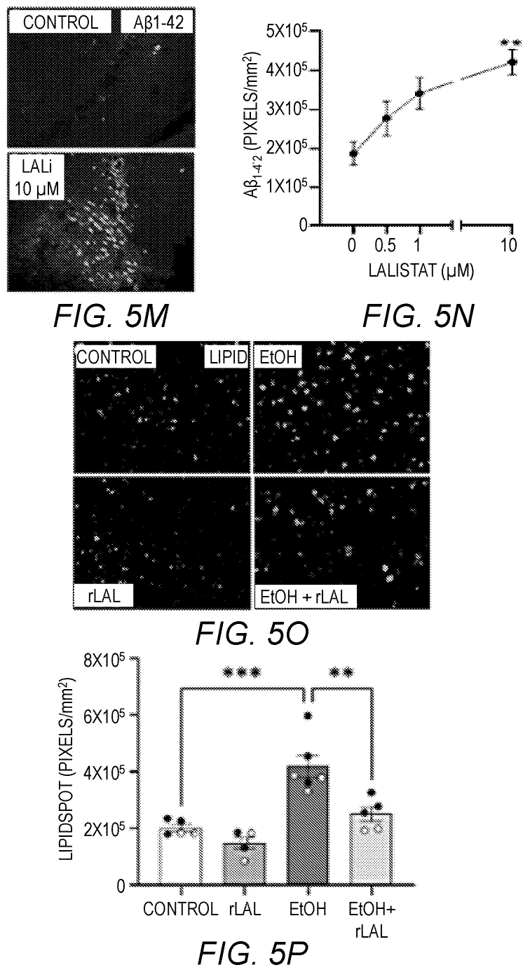

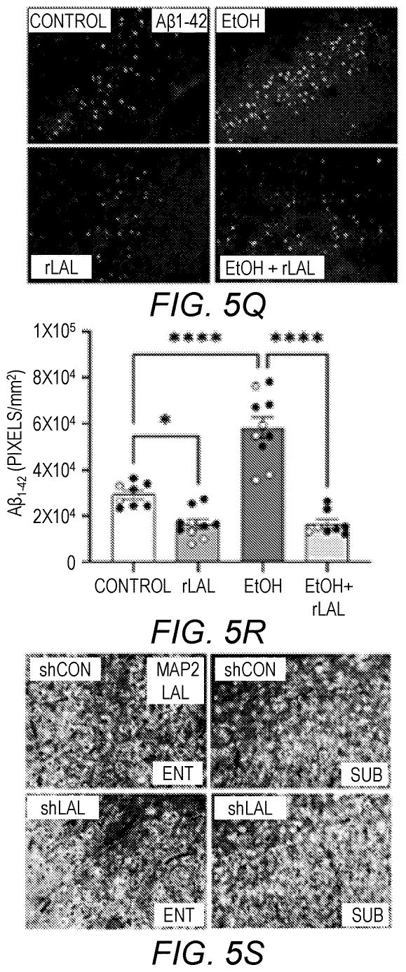

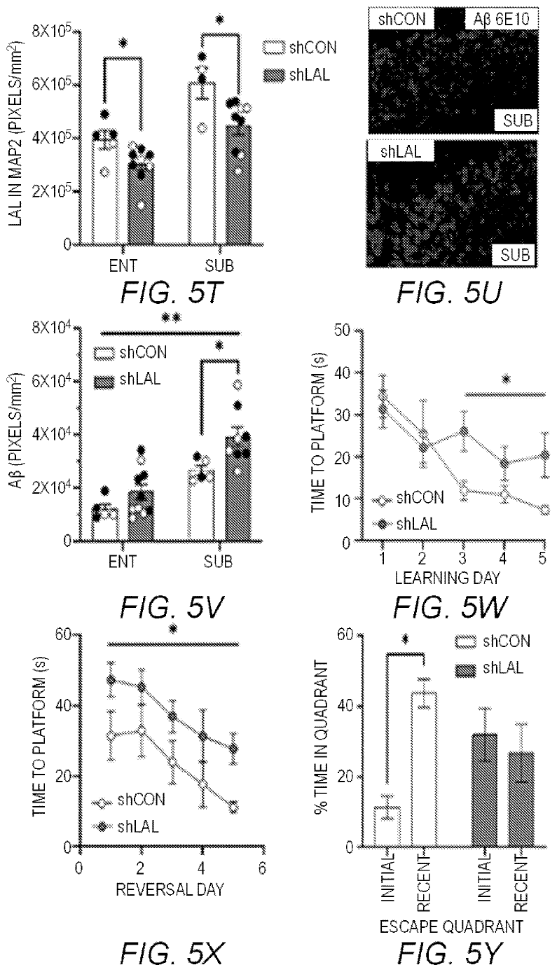

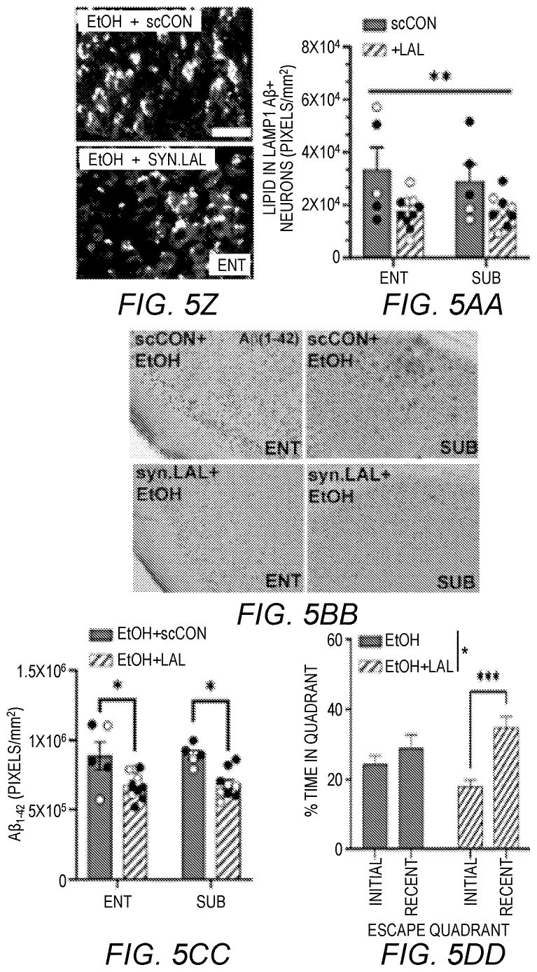

Since LAL is the main lysosomal lipase, the temporal and spatial relationship between LAL and Aβ in AD mice were investigated. Over 60% of LAL was localized to neurons. Neuronal LAL declined with age and preceded accumulation of Aβ AD mouse cortex. In FCX neuronal LAL was significantly lower at 5 months than 3 months of age, prior to measurable increases in Aβ (FIG. 5A). In ENT, lower levels of neuronal LAL were found at 3 months than in FCX, with a slight downward deflection at 5 months and a robust decline by 11 months (FIG. 5B). At 11 months, regions with more LAL maintained lower levels of Aβ (FIGS. 5C-5D). FCX showed robust LAL staining with lower levels of Aβ, while in the subiculum (SUB), where plaques were found, LAL was essentially absent. The ENT, which had intermediate levels of LAL, also had an intermediate level of Aβ. Consistent with WB measurements (FIG. 2J), ethanol and obesity caused reductions in total and neuronal LAL (FIGS. 5E-5H). Ethanol caused nearly 50% reductions in total LAL in the ENT and SUB (FIG. 5F), with similar magnitude losses within neurons in FCX and ENT (FIG. 5G) as well as in obese AD mouse ENT (FIG. 5H). Strong negative correlations were found between Aβ and LAL across all regions measured (FCX, ENT, SUB) with both ethanol and obesity (FIGS. 5I-5J). To determine if LAL loss can cause Aβ accumulation, HEBSC from 3×Tg-AD mice were utilized. Inhibition of LAL activity with LAListat (LALi) caused a concentration-dependent increase in cytosolic lipid levels (FIGS. 5K-5L) and intraneuronal Aβ in the ENT (FIGS. 5M-5N). Induction of master lysosomal transcription factor TFEB with genistein (FIGS. 11A-11B) and GLP-1/GIP signaling with DA4-JC (FIGS. 11C-11D) did not abolish ethanol-induced increases in cytosolic lipid. However, addition of recombinant LAL/sebelipase (rLAL) blocked the 2-fold increase in cytosolic lipid caused by ethanol (FIGS. 5O-5P). Concomitantly, rLAL reduced baseline Aβ levels by 50% and abolished the 2-fold increase in Aβ caused by ethanol (FIGS. 5Q-5R), indicating a causal role for LAL loss in Aβ accumulation.

Since ex vivo studies supported a causal relationship between LAL loss and Aβ accumulation, determination of whether LAL regulates Aβ and cognitive dysfunction in vivo was ascertained. To determine the impact of neuronal LAL loss, at 8 months of age 3×Tg-AD mice received either PHP.eB.syn.shLAL or PHP.eB.shCON injection. Assessment of learning, memory, and cognitive flexibility began at 11 months of age. PHP.eB.syn.shLAL reduced neuronal LAL in the ENT and SUB (FIGS. 5S-5T, F_{1,23}=23.98, p<0.001), resulting in increases in total lipid (FIGS. 11E-11F), neuronal lipid (FIGS. 11G-11H), and Aβ (FIGS. 5U-5V), indicating a causal relationship between LAL loss and Aβ in vivo. Accordingly, the level of Aβ was strongly correlated with neuronal lipid within Aβ+ neurons across regions (FIG. 11I, R=0.69, **p<0.0001). Mice with neuronal LAL knockdown showed significant deficits in learning in the Morris water maze (FIG. 5W) as well as impaired reversal learning and cognitive flexibility (FIGS. 5X-5Y). To determine if LAL overexpression could improve ethanol-induced enhancement of AD pathology, mice received PHP.eB.syn.WPRE.LAL or PHP.eB.scCON injection at 8 months, followed by chronic ethanol from 9 to 11 months. PHP.eB.syn. WPRE.LAL caused robust overexpression of LAL in 3×Tg-AD cortex and hippocampus (FIG. 11J) as well a reduction in cytosolic lipid (FIGS. 11K-11L), NLL (FIGS. 5Z-5AA), and intraneuronal Aβ in ENT and subiculum (FIGS. 5BB-5CC). This was associated with an improvement in cognitive flexibility (FIG. 5DD) without any changes in spatial learning (FIGS. 11M-11N). Thus, loss of neuronal LAL drives Aβ accumulation and cognitive decline in vivo.

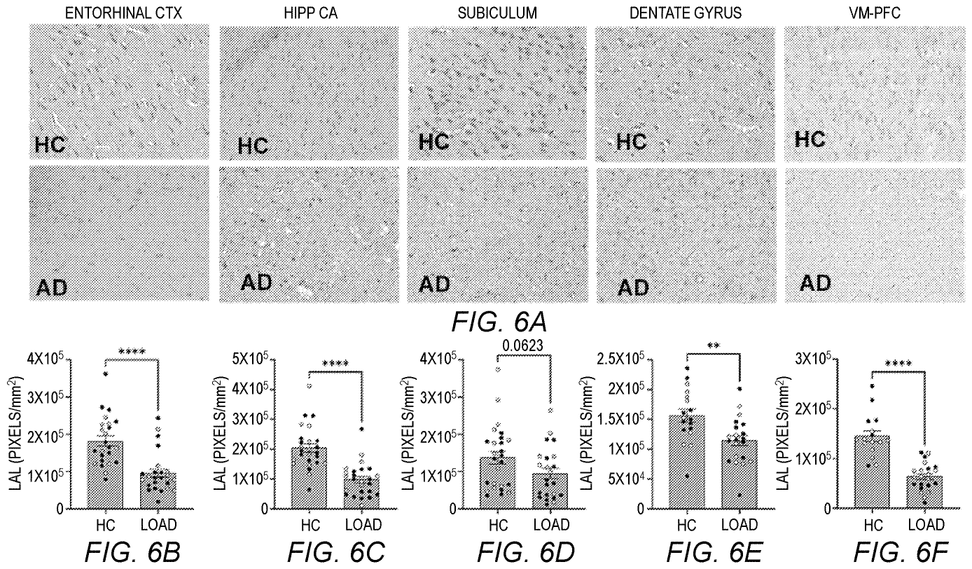

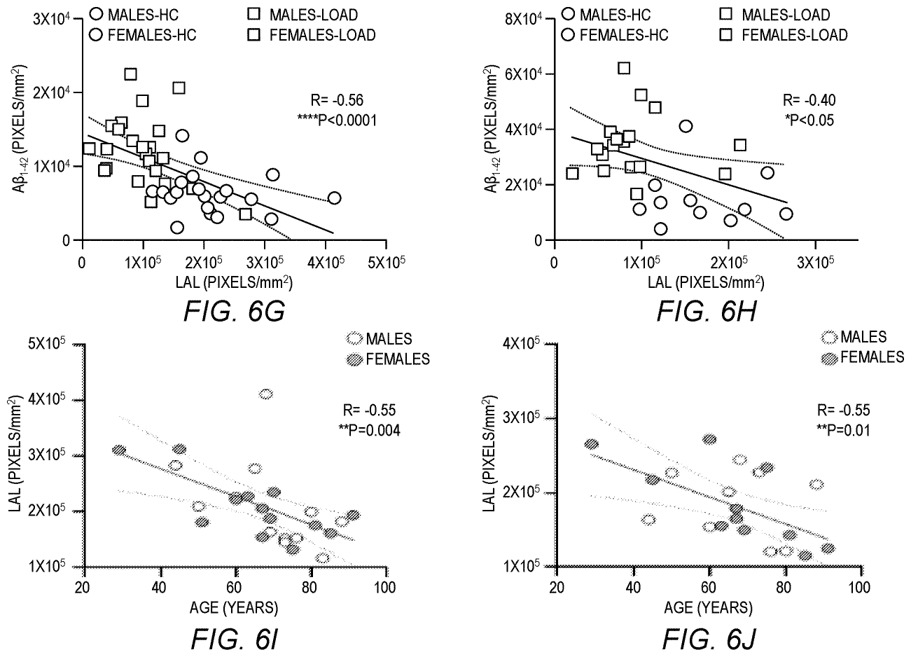

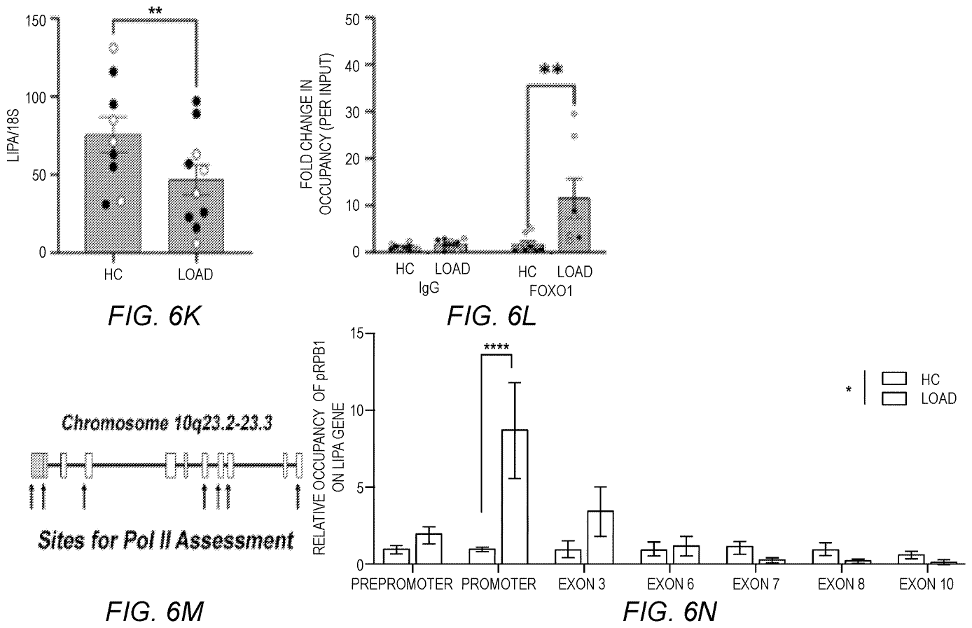

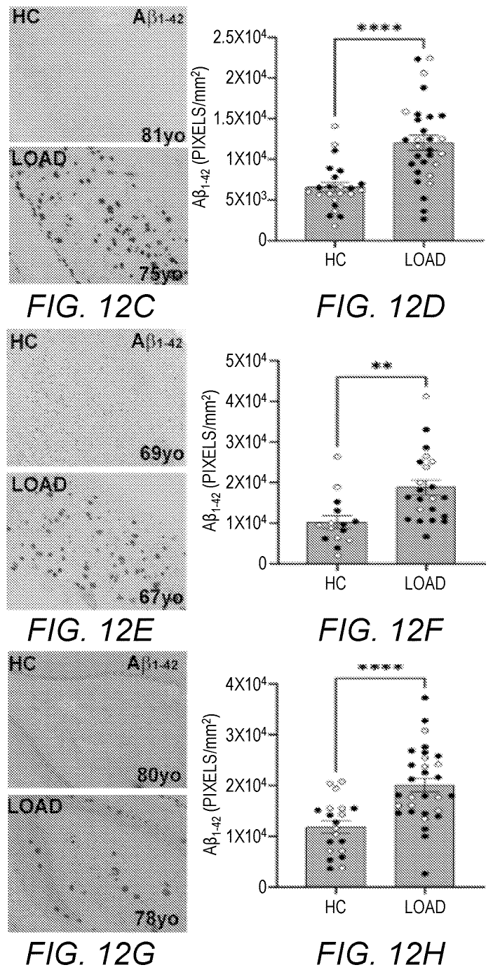

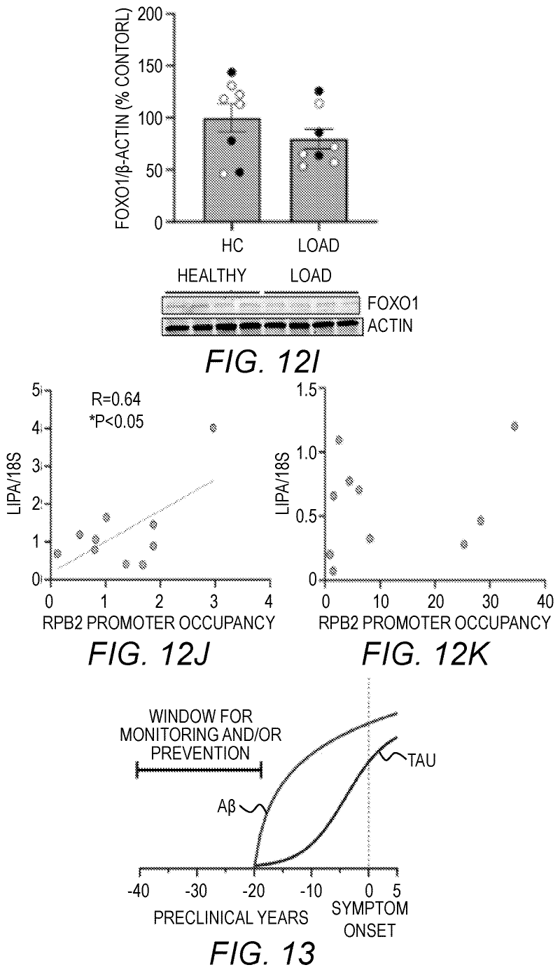

LAL is lost in healthy human aging and LOAD with elongating polymerase pausing. To determine if LAL loss occurs in LOAD, next LAL was assessed in postmortem human LOAD brain and age-matched healthy controls (HC). LAL was found in neurons in ENT, hippocampal CA regions, subiculum, dentate gyrus, and vm-PFC (FIG. 6A, top). However, in human LOAD brain, LAL levels were much lower and clear neuronal morphology was lost (FIG. 6A, bottom). Robust reductions in LAL were found in the ENT (47%, FIG. 6B), CA (70%, FIG. 6C), subiculum (56%, FIG. 6D), dentate (27%, FIG. 5E), and the vm-PFC (62%, FIG. 6F). As expected, Aβ₁₋₄₂ was increased in these same regions in LOAD (FIGS. 12A-12H). Strong negative correlations between LAL and Aβ₁₋₄₂ were also found across all subjects in CA1 (FIG. 6G, R=− 0.60, ****p<0.0001) and ENT (FIG. 6H, R=−0.5, **p<0.0015). Similar to rodents, in healthy subjects LAL protein declined with age, with strong negative correlations seen in CA1 (FIG. 6I, R=−0.55, **p<0.004) and ENT (FIG. 6J, R=−0.55, **p<0.01). Expression of the LAL gene (LIPA) was reduced in LOAD HIPP (FIG. 6K). Promoter occupancy of its main transcription factor FOXO1 was greatly increased, though total FOXO1 protein levels were unchanged (FIG. 6L, FIGS. 12A-12H). Assessment of the localization of active RNA polymerase II (p-RBP1) across the LIPA gene body (FIG. 6M) found enhanced binding at the LIPA promoter region and exon 3, with a reduction below HC levels at later exons (FIG. 6N). In healthy subjects, LIPA gene expression was positively correlated with p-RBP1 promoter occupancy (FIG. 12J, R=0.64, *p<0.05), while in LOAD, no association between p-RBP1 promoter occupancy and gene expression was found (FIG. 12K). These findings are consistent with pausing of elongating RNA polymerase, resulting in reduced transcription of LAL and subsequent promotion of Aβ pathology.

### Discussion

In this report, by comparing two distinct midlife LOAD risk factors, heavy alcohol use and obesity, this example found that the loss of neuronal LAL precedes and promotes Aβ pathology and cognitive deficits by promoting the accumulation of lipid in neuronal lysosomes. Evidence for this was found in vivo (AD and WT mice), ex vivo, and in human LOAD brain. Increased NLL reduced localization of Aβ to lysosomes, which may include both disruption in endolysosomal fusion and reduced lysosomal efficiency, as supported by reduced lysotracker and downregulation of vATPases. This would be consistent with recent work finding a reduction of autolysosomal acidification precedes intraneuronal Aβ accumulation and subsequent plaque formation¹⁴. Interestingly, genistein, an agonist of the master lysosomal transcription factor TFEB, did not reduce lipid accumulation caused by alcohol. However, LAL supplementation normalized lysosomal lipid levels and Aβ both in vitro and in vivo. This suggests that normalizing lysosomal acidification alone may not be sufficient to prevent disease progression. Neuronal knock-down of LAL in AD mice increased Aβ accumulation and caused deficits in memory and cognitive flexibility, which are features of LOAD. Neuronal LAL overexpression blunted increases in intraneuronal and extracellular Aβ pathology and improved cognitive flexibility. The loss of LAL in human postmortem LOAD subjects, who did not have significant alcohol use or obesity, suggests this is a fundamental feature of LOAD that extends beyond these two risk factors.

Aging is requisite for the development of LOAD. Studying LOAD in vivo is complicated by the fact that rodents do not naturally develop robust LOAD pathology. Therefore, nearly all AD models include genetic mutations associated with inherited AD or the expression of human amyloid and tau genes. Though the 3×Tg-AD model features lifelong expression of human familial AD transgenes, pathology emerges slowly with aging. This work finds that the loss of LAL represents the loss of a key resilience mechanism against Aβ accumulation. This was supported by findings in WT mice. NLL increased greatly with age WT mice along with an age-related loss in LAL and corresponding amyloid accumulation in the ENT, which mirrors findings in healthy humans. This suggests that LAL loss with a subsequent increase in NLL is an age-related phenomenon that produces a neuro-environment that is vulnerable to AD. Work in human LOAD hippocampus found polymerase pausing at the LAL gene which was associated with reduced LAL transcription. It is believed this is the first report of RNA polymerase pausing mediating LOAD pathology and is consistent with recent findings of Pol II insufficiency the aged mouse liver³⁶. Removing the brake on the transcription of LAL and other genes could serve as a future therapeutic approach, and the global role of RNA pausing in LOAD is a focus of ongoing studies. Further, microglia become increasingly polarized to proinflammatory state with aging and LOAD^{37,38}. The finding that chemogenetic inhibition of microglia prevents increases in NLL implicates microglia as regulators of neuronal lipid metabolism and suggests aged microglia can promote AD pathology by inducing neuronal lipid changes. Future studies will investigate the mechanisms by which microglia regulate neuronal lipid metabolism. Together, this work finds an age-related loss in neuronal LAL in combination with risk factors such as heavy alcohol use, obesity, or perhaps other lipid-related AD genetics, can result in the emergence of AD pathology.

These findings are consistent with recent single cell transcriptomic studies that also find reduced expression of LAL in excitatory neurons. Mining of published datasets from Zhou et al and Mathys et al found that LAL is reduced by ~10% in cortical neurons and ~14% in excitatory neurons respectively, which is similar to the level of reduction found in mice after chronic alcohol and obesity. In addition to reduced LAL, both alcohol and obesity reduced the expression of lipid efflux transporters (ABCG1 and ABCA1) which could also promote intracellular lipid accumulation. Polymorphisms in ABCA1 and other lipid efflux transporters such as ABCA2 and ABCA7 are associated with increased risk for LOAD, and expression of ABCA1 was reported to be reduced in excitatory neurons, further implicating a role for NLL in LOAD. Further, both alcohol and obesity altered expression of lipid coating PLIN proteins in a manner consistent with reduced lysosomal degradation of lipids. Expression of PLIN4, which can prevent lysosomal degradation of cytosolic lipid droplets, was increased with age in WT mice and by both ethanol and obesity in 3×Tg-AD mice, whereas PLIN2 and PLIN3, which promote lysosomal degradation, were decreased. Together, this suggests that reductions in lipid efflux in addition to lipophagy may result in NLL and reduced degradation of intraneuronal amyloid. Therefore, multiple approaches could be valuable in lowering the intracellular neuronal lipid burden to slow or prevent LOAD progression.

Beyond LOAD, lysosomal lipid accumulation and subsequent neuronal injury is found in lysosomal storage disorders such as Nieman Pick Type C^{43,44}. Severe loss of LAL is found in LAL deficiency (LAL-D) disorder, an autosomal inborn error of metabolism with widespread accumulation of lysosomal lipid in multiple organs. Individuals with these conditions typically have a shortened lifespan. Therefore, it is unknown whether they would be at increased risk for AD. LAL replacement therapy with sebelipase alpha/rLAL was recently approved for the treatment of LAL-D. This work suggests that increasing levels of neuronal LAL could have preventative or therapeutic value for LOAD. In summary, the loss of neuronal LAL with age is found to promote accumulation of neuronal lysosomal lipid and prevent lysosomal degradation of amyloid within neurons.

The following references are related to Example 1.

### REFERENCES

1 Pedditzi, E., Peters, R. & Beckett, N. The risk of overweight/obesity in mid-life and late life for the development of dementia: a systematic review and meta-analysis of longitudinal studies. Age Ageing 45, 14-21 (2016). https://doi.org: 10.1093/ageing/afv151
2 Xu, W. et al. Meta-analysis of modifiable risk factors for Alzheimer's disease. Journal of neurology, neurosurgery, and psychiatry 86, 1299-1306 (2015). https://doi.org: 10.1136/jnnp-2015-310548
3 Li, J. Q. et al. Risk factors for predicting progression from mild cognitive impairment to Alzheimer's disease: a systematic review and meta-analysis of cohort studies. Journal of neurology, neurosurgery, and psychiatry 87, 476-484 (2016). https://doi.org: 10.1136/jnnp-2014-310095
4 Harwood, D. G. et al. The effect of alcohol and tobacco consumption, and apolipoprotein E genotype, on the age of onset in Alzheimer's disease. International journal of geriatric psychiatry 25, 511-518 (2010). https://doi.org: 10.1002/gps.2372
5 Schwarzinger, M. et al. Contribution of alcohol use disorders to the burden of dementia in France 2008-13: a nationwide retrospective cohort study. The Lancet. Public health 3, e124-e132 (2018). https://doi.org: 10.1016/S2468-2667(18) 30022-7
6 Long, J. M. & Holtzman, D. M. Alzheimer Disease: An Update on Pathobiology and Treatment Strategies. Cell 179, 312-339 (2019). https://doi.org: 10.1016/j.cell.2019.09.001
7 Barrachina, M., Maes, T., Buesa, C. & Ferrer, I. Lysosome-associated membrane protein 1 (LAMP-1) in Alzheimer's disease. Neuropathol Appl Neurobiol 32, 505-516 (2006). https://doi.org: 10.1111/j.1365-2990.2006.00756.x
8 Tucker, A. E., Alicea Pauneto, C. d. M., Barnett, A. M. & Coleman, L. G. Chronic Ethanol Causes Persistent Increases in Alzheimer's Tau Pathology in Female 3×Tg-AD Mice: A Potential Role for Lysosomal Impairment. Frontiers in Behavioral Neuroscience 16, 886634 (2022). https://doi.org: 10.3389/fnbeh.2022.886634
9 Almeida, M. F., Bahr, B. A. & Kinsey, S. T. Endosomal-lysosomal dysfunction in metabolic diseases and Alzheimer's disease. International review of neurobiology 154, 303-324 (2020). https://doi.org: 10.1016/bs.irn.2020.02.012
10 Wolfe, D. M. et al. Autophagy failure in Alzheimer's disease and the role of defective lysosomal acidification. The European journal of neuroscience 37, 1949-1961 (2013). https://doi.org: 10.1111/ejn.12169
11 Nixon, R. A. The role of autophagy in neurodegenerative disease. Nature medicine 19, 983-997 (2013). https://doi.org: 10.1038/nm.3232
12 Peric, A. & Annaert, W. Early etiology of Alzheimer's disease: tipping the balance toward autophagy or endosomal dysfunction? Acta neuropathologica 129, 363-381 (2015). https://doi.org: 10.1007/s00401-014-1379-7
13 Ginsberg, S. D. et al. Microarray analysis of hippocampal CA1 neurons implicates early endosomal dysfunction during Alzheimer's disease progression. Biological psychiatry 68, 885-893 (2010). https://doi.org: 10.1016/j.biopsych.2010.05.030
14 Lee, J. H. et al. Faulty autolysosome acidification in Alzheimer's disease mouse models induces autophagic build-up of Abeta in neurons, yielding senile plaques. Nature neuroscience 25, 688-701 (2022). https://doi.org: 10.1038/s41593-022-01084-8
15 Huang, Y. & Mahley, R. W. Apolipoprotein E: structure and function in lipid metabolism, neurobiology, and Alzheimer's diseases. Neurobiology of disease 72 Pt A, 3-12 (2014). https://doi.org: 10.1016/j.nbd.2014.08.025
16 Eid, A., Mhatre, I. & Richardson, J. R. Gene-environment interactions in Alzheimer's disease: A potential path to precision medicine. Pharmacology & therapeutics 199, 173-187 (2019). https://doi.org: 10.1016/j.pharmthera.2019.03.005
17 Katzov, H. et al. Genetic variants of ABCA1 modify Alzheimer disease risk and quantitative traits related to beta-amyloid metabolism. Hum Mutat 23, 358-367 (2004). https://doi.org: 10.1002/humu.20012
18 Mace, S. et al. ABCA2 is a strong genetic risk factor for early-onset Alzheimer's disease. Neurobiology of disease 18, 119-125 (2005). https://doi.org: 10.1016/j.nbd.2004.09.011
19 Yu, J. T. et al. Evidence-based prevention of Alzheimer's disease: systematic review and meta-analysis of 243 observational prospective studies and 153 randomised controlled trials. Journal of neurology, neurosurgery, and psychiatry 91, 1201-1209 (2020). https://doi.org: 10.1136/jnnp-2019-321913
20 Barnett, A. M. et al. Adolescent Binge Alcohol Enhances Early Alzheimer's Disease Pathology in Adulthood Through Proinflammatory Neuroimmune Activation. Frontiers in pharmacology 13, 884170 (2022). https://doi.org: 10.3389/fphar.2022.884170
21 Oddo, S., Caccamo, A., Kitazawa, M., Tseng, B. P. & LaFerla, F. M. Amyloid deposition precedes tangle formation in a triple transgenic model of Alzheimer's disease. Neurobiology of aging 24, 1063-1070 (2003).
22 Oddo, S. et al. Triple-transgenic model of Alzheimer's disease with plaques and tangles: intracellular Abeta and synaptic dysfunction. Neuron 39, 409-421 (2003).
23 Belfiore, R. et al. Temporal and regional progression of Alzheimer's disease-like pathology in 3×Tg-AD mice. Aging cell 18, e12873 (2019). https://doi.org: 10.1111/acel.12873
24 Schmidt, W., Popham, R. E. & Israel, Y. Dose-specific effects of alcohol on the lifespan of mice and the possible relevance to man. Br J Addict 82, 775-788 (1987). https://doi.org: 10.1111/j.1360-0443.1987.tb01545.x
25 Coleman, L. G., Jr., He, J., Lee, J., Styner, M. & Crews, F. T. Adolescent binge drinking alters adult brain neurotransmitter gene expression, behavior, brain regional volumes, and neurochemistry in mice. Alcoholism, clinical and experimental research 35, 671-688 (2011). https://doi.org: 10.1111/j.1530-0277.2010.01385.x
26 Thijssen, E. H. et al. Diagnostic value of plasma phosphorylated tau181 in Alzheimer's disease and frontotemporal lobar degeneration. Nature medicine (2020). https://doi.org: 10.1038/s41591-020-0762-2
27 Karikari, T. K. et al. Blood phosphorylated tau 181 as a biomarker for Alzheimer's disease: a diagnostic performance and prediction modelling study using data from four prospective cohorts. The Lancet. Neurology 19, 422-433 (2020). https://doi.org: 10.1016/S1474-4422 (20) 30071-5
28 Repnik, U., Cesen, M. H. & Turk, B. The endolysosomal system in cell death and survival. Cold Spring Harb Perspect Biol 5, a008755 (2013). https://doi.org: 10.1101/cshperspect.a008755
29 Hansen, T. E. & Johansen, T. Following autophagy step by step. BMC Biol 9, 39 (2011). https://doi.org: 10.1186/1741-7007-9-39
30 Settembre, C. et al. TFEB links autophagy to lysosomal biogenesis. Science 332, 1429-1433 (2011). https://doi.org: 10.1126/science. 1204592
31 Sheng, J. G. et al. Lipopolysaccharide-induced-neuroinflammation increases intracellular accumulation of amyloid precursor protein and amyloid beta peptide in APPswe transgenic mice. Neurobiology of disease 14, 133-145 (2003). https://doi.org: 10.1016/s0969-9961 (03) 00069-x
32 Crews, F. T., Zou, J. & Coleman, L. G., Jr. Extracellular microvesicles promote microglia-mediated pro-inflammatory responses to ethanol. Journal of neuroscience research 99, 1940-1956 (2021). https://doi.org: 10.1002/jnr.24813
33 Coleman, L. G., Jr., Zou, J. & Crews, F. T. Microglial depletion and repopulation in brain slice culture normalizes sensitized proinflammatory signaling. Journal of neuroinflammation 17, 27 (2020). https://doi.org: 10.1186/s12974-019-1678-y
34 Deczkowska, A. et al. Disease-Associated Microglia: A Universal Immune Sensor of Neurodegeneration. Cell 173, 1073-1081 (2018). https://doi.org: 10.1016/j.cell.2018.05.003
35 Keren-Shaul, H. et al. A Unique Microglia Type Associated with Restricting Development of Alzheimer's Disease. Cell 169, 1276-1290 e1217 (2017). https://doi.org: 10.1016/j.cell.2017.05.018
36 Gyenis, A. et al. Genome-wide RNA polymerase stalling shapes the transcriptome during aging. Nature genetics 55, 268-279 (2023). https://doi.org: 10.1038/s41588-022-01279-6
37 Prater, K. E. et al. Human microglia show unique transcriptional changes in Alzheimer's disease. Nat Aging 3, 894-907 (2023). https://doi.org: 10.1038/s43587-023-00424-y
38 Marschallinger, J. et al. Lipid-droplet-accumulating microglia represent a dysfunctional and proinflammatory state in the aging brain. Nature neuroscience 23, 194-208 (2020). https://doi.org: 10.1038/s41593-019-0566-1
39 Zhou, Y. et al. Human and mouse single-nucleus transcriptomics reveal TREM2-dependent and TREM2-independent cellular responses in Alzheimer's disease. Nature medicine 26, 131-142 (2020). https://doi.org: 10.1038/s41591-019-0695-9
40 Mathys, H. et al. Single-cell transcriptomic analysis of Alzheimer's disease. Nature 570, 332-337 (2019). https://doi.org: 10.1038/s41586-019-1195-2
41 Han, X. et al. Plin4-Dependent Lipid Droplets Hamper Neuronal Mitophagy in the MPTP/p-Induced Mouse Model of Parkinson's Disease. Front Neurosci 12, 397 (2018). https://doi.org: 10.3389/fnins.2018.00397
42 Kaushik, S. & Cuervo, A. M. Degradation of lipid droplet-associated proteins by chaperone-mediated autophagy facilitates lipolysis. Nature cell biology 17, 759-770 (2015). https://doi.org: 10.1038/ncb3166
43 Liao, G. et al. Cholesterol accumulation is associated with lysosomal dysfunction and autophagic stress in Npc1−/− mouse brain. The American journal of pathology 171, 962-975 (2007). https://doi.org: 10.2353/ajpath.2007.070052
44 Meske, V., Erz, J., Priesnitz, T. & Ohm, T. G. The autophagic defect in Niemann-Pick disease type C neurons differs from somatic cells and reduces neuronal viability. Neurobiology of disease 64, 88-97 (2014). https://doi.org: 10.1016/j.nbd.2013.12.018

### Methods

### Postmortem Human Brain Samples

Brains from human donors were from the New South Wales Brain Tissue Resource Centre (NSW-BTRC) and the Victoria Brain Bank (VBB) in Australia. Human samples were obtained under Ethics Committee Approval Number X11-0107^{1,2}.

### Mouse Models

3×Tg-AD (human APPSwe, tauP301, and Psen1^{tm1Mpm})^{3,4} male and female breeders were obtained from the Jackson Laboratory Mutant Mouse Resource & Research Centers (MMRC). WT mice were bred in house or obtained from the NIA aged mouse colony. Mice were bred and pups weaned at 30 days of age and group-housed with same-sex littermates. Animal protocols were approved by the University of North Carolina at Chapel Hill Institutional Animal Care and Use Committee (IACUC) and were in accordance with NIH regulations (Protocols 20-232.0 and 21-052.0).

### Chronic Alcohol Treatment

3×Tg-AD or WT mice, mice received a single daily intragastric (i.g.) administration of either alcohol (i.e., ethanol) or water (five-days on/two-days off) to mimic human intermittent drinking patterns during midlife (9 months of age) for 5 to 8 weeks. Mice metabolize ethanol ~8× faster than humans 5. Therefore, a dose was given that produces an average blood alcohol concentration (BAC) of ~0.1 mg/dL during the 12 hours of intoxication (5 g/kg/d, i.g., 20% ethanol w/v, peak BAC~280 mg/dL at 1 hour)^{6,7}. No differences in body weight were found between treatment groups. Mice were sacrificed 24 h after final administration and tissue was collected for tissue analyses.

### Western Diet-Induced Midlife Obesity

3×Tg-AD or WT mice were fed either a control complete diet (3.6 kcal/g, 14.8% fat) or a Western diet to induce obesity (4.5 kcal/g, 21.2% fat) from 6 to 11 months of age. Mice were sacrificed and tissue was collected for analysis.

### Microglial Gi DREADD Inhibition

For ex-vivo slice culture experiments HEBSCs from 3×Tg-AD mice were transfected with hM4di into microglia (AAV9.CD68.hM4di for 24 h) as previously reported^{8,9} and then treated with ethanol (100 mM, 4 days)+/−CNO (1 μM). For in vivo studies, heterozygous CX3CR1.Cre^{ERT}.hM4di were treated with tamoxifen (75 mg/kg/d, i.p.) for 5 days during adulthood (12 weeks of age). After 4 weeks to allow for repopulation of peripheral monocytes, mice received either ethanol (5 g/kg/d, i.g.) or water for 10 days +/−CNO (3 mg/kg, 10 hours after ethanol) and sacrificed 24 hours after the last ethanol treatment.

### Perfusion and Tissue Collection for Immunohistochemistry

At the conclusion of each experiment, subjects were sacrificed by transcardial perfusion with 0.1 M phosphate-buffered saline (PBS, pH 7.4), and brains excised and hemisected. On one hemisphere, the cortex and hippocampus were dissected and snap frozen in liquid nitrogen for protein and RNA analyses as reported previously¹⁰. The other hemisphere was drop-fixed in 4.0% paraformaldehyde for immunohistochemical assessments. Coronal sections were cut (40 μm) on a sliding microtome (MICROM HM450; ThermoScientific, Austin, TX), and sections were sequentially collected into well plates and stored at −20° C. in cryoprotectant (30% glycol/30% ethylene glycol in PBS).

### RT-PCR

mRNA was extracted from frozen cortex or hippocampus as reported¹⁰. Briefly, samples were homogenized with Trizol (Invitrogen) and RNA was isolated by chloroform extraction, followed by reverse transcription as described previously 11. SYBR green PCR master mix (Applied Biosystems, Foster City, CA) was used for qRT-PCR analysis. Primer sequences were designed using the National Library of Medicine Primer-BLAST tool or were obtained from PrimerBank database (Table 1) 12-14. Only primers with no predicted non-specific targets and single peak melt curves were used. Genes of interest were normalized to the expression of the reference gene 18S using the cycle threshold (Ct) value of each target gene product. The ΔΔCt method was used to compare relative differences between control and treatment groups, and the ratio by 18S or the percent change relative to 18S were used in analysis.

### Western Blot

Western blot was performed as reported previously^{9,10}. Brain tissue from the cerebral cortex was homogenized in lysis buffer (Tris-HCl, pH 7.5, Sucrose, EDTA, EGTA, 1% Triton X-100, protease, and phosphatase inhibitors). Forty milligrams of protein were loaded into each lane on SDS polyacrylamide gels and were transferred to PVDF membranes. Membranes were washed in TBS and blocked for 1 hour at room temperature (Li-Cor Blocking Solution; 92760001) then were incubated overnight at 4° Celsius with primary antibodies (Table 1). Membranes were washed in TBS with 0.1% Tween-20 (Sigma-Aldrich, St. Louis, MO) then were incubated in the appropriate conjugated secondary antibody (Rockland H & L Pre-absorbed). Membranes were washed again in TBS and visualized using LiCor Image Studio Lite Ver 5.2. Western Blots were analyzed using Image Studio Lite software and each protein of interest was normalized to housekeeping protein GAPDH. GAPDH expression was not affected by ethanol treatment or high-fat diet. The protein of interest was normalized to GAPDH for each sample and the percentage change relative to controls was calculated for each blot.

### Immunohistochemistry (IHC) and Immunofluorescence (IF)

Free-floating sections (40 μm) were washed in 0.1 M PBS, quenched in 0.6% H₂O₂ for 30 minutes to inhibit endogenous peroxidases, then incubated at 70° C. in pH=6.0 1× Citrate Buffer for antigen retrieval. To allow membrane permeabilization, sections were blocked for 1 hour at room temperature in 4% normal serum with 0.1% Triton X-100. Sections were incubated in the relevant primary antibody (please see above) in blocking solution at 4° C. overnight. Negative controls for non-specific binding were done using the same protocol, omitting the primary antibody. The next day sections were incubated at room temperature for one hour with a biotinylated secondary antibody (1:200, Vector Laboratories, Burlingame, CA), washed briefly in PBS, then incubated for 1 hour in avidin-biotin complex (ABC) (Vectastain ABC Kit; Vector Laboratories). Nickel-enhanced diaminobenzidine (Sigma-Aldrich) was used as the chromogen for visualization. Sections were mounted on charged glass slides, allowed to dry, dehydrated in a series of ethanol, and covered using Cytoseal (Fischer). For IF, the day after primary antibody incubation, sections were washed with PBS and incubated for 1 hour at room temperature with the respective Alexa Fluor conjugated secondary antibodies (1:1000; Invitrogen, Carlsbad, Ca, USA). If staining for neutral lipid droplets was being performed, sections were washed with PBS then stained with 1× LipidSpot AlexaFluor 488 (Biotium, San Francisco, CA) in PBS for 20 min. Sections were mounted and cover slips placed with Prolong Gold Anti-Fade mounting medium with DAPI (Invitrogen; Carlsbad, CA, USA). Negative control for non-specific binding was done separately with the exception that the primary antibody was omitted.

The Keyence BZ-X800 all-in-one Microscope was used for representative images and microscopic analysis of tissue. For each region of interest assessed, images were taken on 3-5 identical sections per subject at a representative location, selected using the mouse brain atlas¹⁵. Bregma used by region were: entorhinal cortex and subiculum (−3.52 to −4.04 mm), and CA1 (−2.92 to −3.40 mm). The immunoreactivity (+IR) was quantified in each region using either the BZ-X800 software or ImageJ Analysis Software and reported as +IR pixels/mm².

### Hippocampal-Entorhinal Brain Slice Culture (HEBSC)

HEBSC was performed using techniques routinely used in the laboratory^{2,9,11} Briefly, sections were prepared from either the P7 or adult (6 mo) 3×-Tg-AD hippocampal-entorhinal cortex formation as described by Stoppini et al¹⁶. Initial experiments were done on HEBSC from P7 mice then in HEBSC from adults to ensure developmental differences did not drive findings. Mice were decapitated, brains extracted and the hippocampal-entorhinal complex dissected in Gey's bugger (Sigma-Aldrich). Slices were cut transversely at 375 μm using a McIlwain tissue chopper and placed on Millicell culture inserts (Millipore, PICMORG50, up to 13 slices per insert). Slices acclimated in culture media (MEM plus 24 mM HEPES and Hank's salts, 25% horse serum, 5.5 g/L glucose, 2 mM L-glutamine) in a humified 5% CO₂ incubator for seven days, followed by 12% horse serum for four days, and 6% horse serum until the completion of the experiments.

### Behavior

Open field. Exploratory activity in a novel environment was used to insure none of the mice had overt motor impairment or were notably hypoactive (a sign of possible health issues) before starting evaluation in the Morris water maze. This assessed by a one-hour trial in an open field chamber (41 cm×41 cm×30 cm) crossed by a grid of photobeams (VersaMax system, AccuScan Instruments). Counts were taken of the number of photobeams broken during the trial in 5-min intervals, with separate measures for locomotor activity (total distance traveled) and vertical rearing movements. Time spent in the center region was used as an index of anxiety-like behavior.

Morris water maze. The water maze was used to evaluate spatial and reversal learning, swimming ability, and vision. The water maze consisted of a large circular pool (diameter=122 cm) partially filled with water (45 cm deep, 24-26° C.), located in a room with numerous visual cues. The procedure involved a visible platform test, acquisition in the hidden platform task, and a test for reversal learning (an index of cognitive flexibility). Visible platform test. Each mouse was given 4 trials per day, across 2 days, to swim to an escape platform cued by a patterned cylinder extending above the surface of the water. For each trial, the mouse was placed in the pool at 1 of 4 possible locations (randomly ordered), and then given 60 sec to find the visible platform. If the mouse found the platform, the trial ended, and the animal was allowed to remain 10 sec on the platform before the next trial began. If the platform was not found, the mouse was placed on the platform for 10 sec, and then given the next trial. Measures were taken of latency to find the platform and swimming speed via an automated tracking system (Noldus Ethovision). Acquisition and reversal learning in a hidden platform task. Following the visible platform task, mice were tested for their ability to find a submerged, hidden escape platform (diameter=12 cm). Each mouse was given 4 trials per day, with 1 min per trial, to swim to the hidden platform. The criterion for learning was an average group latency of 15 sec or less to locate the platform. Mice were tested until the group reached criterion, with a maximum of 9 days of testing. When the group reached criterion (on day 5 in the present study), mice were given a one-min probe trial in the pool with the platform removed. Selective quadrant search was evaluated by measuring percent time in the quadrant where the platform (the target) had been placed during training, versus the opposite quadrant, and number of crosses over the target location where the platform had been placed, versus the corresponding area in the opposite quadrant. Following the acquisition phase, mice were tested for reversal learning, using the same procedure as described above. In this phase, the hidden platform was re-located to the opposite quadrant in the pool. As before, measures were taken of latency to find the platform. On day 6 of testing, the platform was removed from the pool, and the group was given a probe trial to evaluate reversal learning.

### Chromatin Immunoprecipitation (CHIP).

Chromatin immunoprecipitation was performed as previously described by the laboratory 17-20 Briefly, postmortem human hippocampal tissue from CON and AD individuals was homogenized, cross-linked with 1.0% methanol-free formaldehyde, quenched with 1.0 M glycine, lysed with lysis buffer (1.0% [v/v] SDS, 10 mM EDTA, 50 mM Tris-HCl [pH 8.0]), and chromatin sheared to fragments of <1000 bp on a Covaris ME220. Input DNA fractions were removed from the sheared chromatin to be processed separately and the remaining sheared chromatin was incubated overnight at 4° C. with an antibody against rabbit FoxO1, rabbit phospho-Rpb1, or the negative control Rabbit IgG. Protein A Dynabeads were added and rotated at 4° C. for 1 hr followed by five washes in ChIP wash buffer. Both immunoprecipitated DNA and input DNA were eluted in 10% (w/v) Chelex by boiling at 95° C. for 10 min followed by centrifugation. ChIP-enriched DNA was analyzed using qPCR with SSOAdvanced Universal SYBR Green Supermix (Bio-Rad, Berkeley, CA) using primers designed against regions of the LIPA gene. The ΔΔCt method was used to determine fold occupancy relative to control and was normalized to the input DNA fraction.

### Statistical Analyses

The specific statistical test used is noted for each assessment above and were performed in GraphPad Prism™. For preplanned orthogonal contrasts, t-tests were used. For age-matched assessments paired t-tests were employed. 1-way or 2-way ANOVAs were used for multiple-group assessments. Dunnett's or Sidak's post-tests were used for ANOVAs when appropriate. Outliers were detected using the Grubb's test.

The following references are related to the methods of Example 1:
1 Vetreno, R. P., Qin, L., Coleman, L. G., Jr. & Crews, F. T. Increased Toll-like Receptor-MyD88-NFkappaB-Proinflammatory neuroimmune signaling in the orbitofrontal cortex of humans with alcohol use disorder. Alcoholism, clinical and experimental research 45, 1747-1761 (2021). https://doi.org: 10.1111/acer.14669
2 Coleman, L. G., Jr., Zou, J. & Crews, F. T. Microglial-derived miRNA let-7 and HMGB1 contribute to ethanol-induced neurotoxicity via TLR7. Journal of neuroinflammation 14, 22 (2017). https://doi.org: 10.1186/s12974-017-0799-4
3 Oddo, S. et al. Triple-transgenic model of Alzheimer's disease with plaques and tangles: intracellular Abeta and synaptic dysfunction. Neuron 39, 409-421 (2003).
4 Li, Y. D. et al. Activation of hypothalamic-enhanced adult-born neurons restores cognitive and affective function in Alzheimer's disease. Cell Stem Cell 30, 415-432 e416 (2023). https://doi.org: 10.1016/j.stem.2023.02.006
Schmidt, W., Popham, R. E. & Israel, Y. Dose-specific effects of alcohol on the lifespan of mice and the possible relevance to man. Br J Addict 82, 775-788 (1987). https://doi.org: 10.1111/j.1360-0443.1987.tb01545.x
6 Coleman, L. G., Jr., He, J., Lee, J., Styner, M. & Crews, F. T. Adolescent binge drinking alters adult brain neurotransmitter gene expression, behavior, brain regional volumes, and neurochemistry in mice. Alcoholism, clinical and experimental research 35, 671-688 (2011). https://doi.org: 10.1111/j.1530-0277.2010.01385.x
7 Coleman, L. G., Jr., Liu, W., Oguz, I., Styner, M. & Crews, F. T. Adolescent binge ethanol treatment alters adult brain regional volumes, cortical extracellular matrix protein and behavioral flexibility. Pharmacol Biochem Behav 116, 142-151 (2014). https://doi.org: 10.1016/j.pbb.2013.11.021
8 Coleman, L. G., Jr., Zou, J. & Crews, F. T. Microglial depletion and repopulation in brain slice culture normalizes sensitized proinflammatory signaling. Journal of neuroinflammation 17, 27 (2020). https://doi.org: 10.1186/s12974-019-1678-y
9 Zou, J. et al. Ethanol Induces Secretion of Proinflammatory Extracellular Vesicles That Inhibit Adult Hippocampal Neurogenesis Through G9a/GLP-Epigenetic Signaling. Frontiers in immunology 13 (2022). https://doi.org: 10.3389/fimmu.2022.866073
10 Barnett, A. M. et al. Adolescent Binge Alcohol Enhances Early Alzheimer's Disease Pathology in Adulthood Through Proinflammatory Neuroimmune Activation. Frontiers in pharmacology 13, 884170 (2022). https://doi.org: 10.3389/fphar.2022.884170
11 Qin, L. et al. TRAIL Mediates Neuronal Death in AUD: A Link between Neuroinflammation and Neurodegeneration. International journal of molecular sciences 22, 2547 (2021).
12 Spandidos, A., Wang, X., Wang, H. & Seed, B. PrimerBank: a resource of human and mouse PCR primer pairs for gene expression detection and quantification. Nucleic acids research 38, D792-799 (2010). https://doi.org: 10.1093/nar/gkp1005
13 Spandidos, A. et al. A comprehensive collection of experimentally validated primers for Polymerase Chain Reaction quantitation of murine transcript abundance. BMC genomics 9, 633 (2008). https://doi.org: 10.1186/1471-2164-9-633
14 Wang, X. & Seed, B. A PCR primer bank for quantitative gene expression analysis. Nucleic acids research 31, e154 (2003). https://doi.org: 10.1093/nar/gng154
15 Paxinos, G. & Franklin, K. B. J. The mouse brain in stereotaxic coordinates. Compact 2nd edn, (Elsevier Academic Press, 2004).
16 Stoppini, L., Buchs, P. A. & Muller, D. A simple method for organotypic cultures of nervous tissue. J Neurosci Methods 37, 173-182 (1991).
17 Crews, F. T., Fisher, R. P., Qin, L. & Vetreno, R. P. HMGB1 neuroimmune signaling and REST-G9a gene repression contribute to ethanol-induced reversible suppression of the cholinergic neuron phenotype. Mol Psychiatry (2023). https://doi.org: 10.1038/s41380-023-02160-6
18 Crews, F. T. & Vetreno, R. P. Cholinergic REST-G9a gene repression through HMGB1-TLR4 neuroimmune signaling regulates basal forebrain cholinergic neuron phenotype. Frontiers in molecular neuroscience 15, 992627 (2022). https://doi.org: 10.3389/fnmol.2022.992627
19 Crews, F. T., Fisher, R., Deason, C. & Vetreno, R. P. Loss of Basal Forebrain Cholinergic Neurons Following Adolescent Binge Ethanol Exposure: Recovery With the Cholinesterase Inhibitor Galantamine. Front Behav Neurosci 15, 652494 (2021). https://doi.org: 10.3389/fnbeh.2021.652494
20 Vetreno, R. P. et al. Neuroimmune and epigenetic involvement in adolescent binge ethanol-induced loss of basal forebrain cholinergic neurons: Restoration with voluntary exercise. Addict Biol 25, e12731 (2020). https://doi.org: 10.1111/adb.12731

### Example 2—LAL-PET

Early identification of individuals at risk for sporadic AD could enable reduction in disease prevalence. AD is the most common form of dementia with over 153 million cases expected worldwide in 205028. Over 90% of AD cases occur later in life and have no clear etiology, making risk prediction on an individual level difficult. However, this late onset or sporadic AD (LOAD) features a preclinical period prior to symptom onset1 that represents a period for potential intervention (FIG. 13). During this preclinical period exposures and interventions can increase or reduce AD risk2-5. For instance, a combination of 4-5 protective activities can reduce risk by up to 40-60% 36. Once symptoms begin, patients already have notable amyloid and tau pathology. A marker of disease that predicts future AD pathogenesis prior to measurable amyloid (Aβ) pathology would warrant targeted interventions or early initiation of Aβ-targeting or other therapies to potentially increase their efficacy. Thus, there is an urgent need for imaging techniques capable of identifying people at increased risk for AD years before symptom onset.

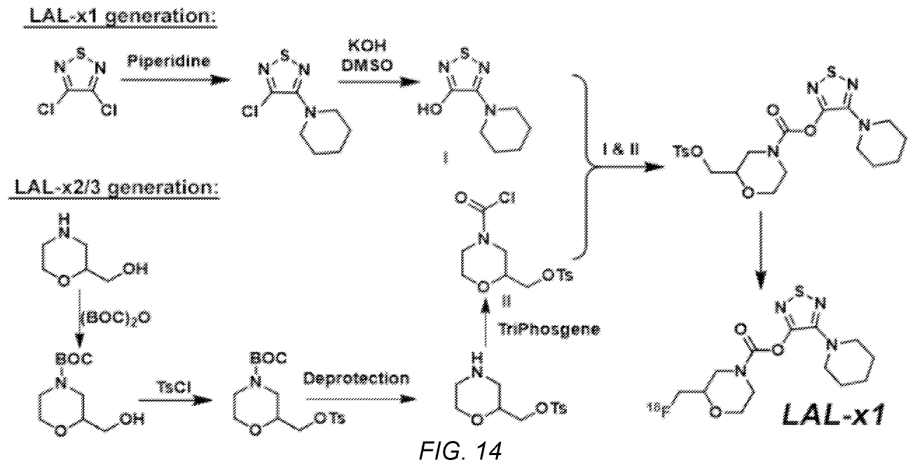

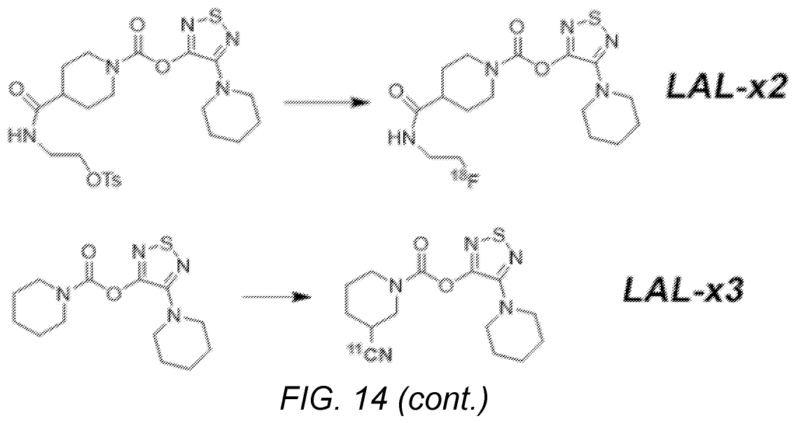

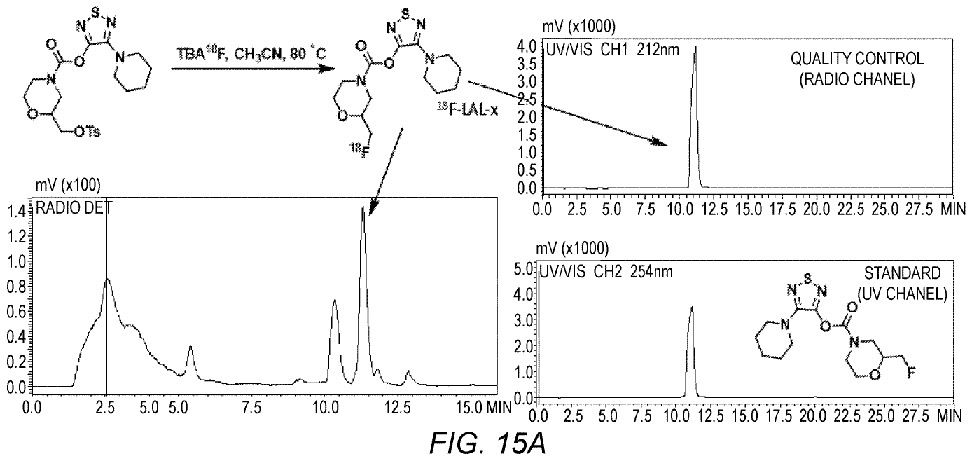

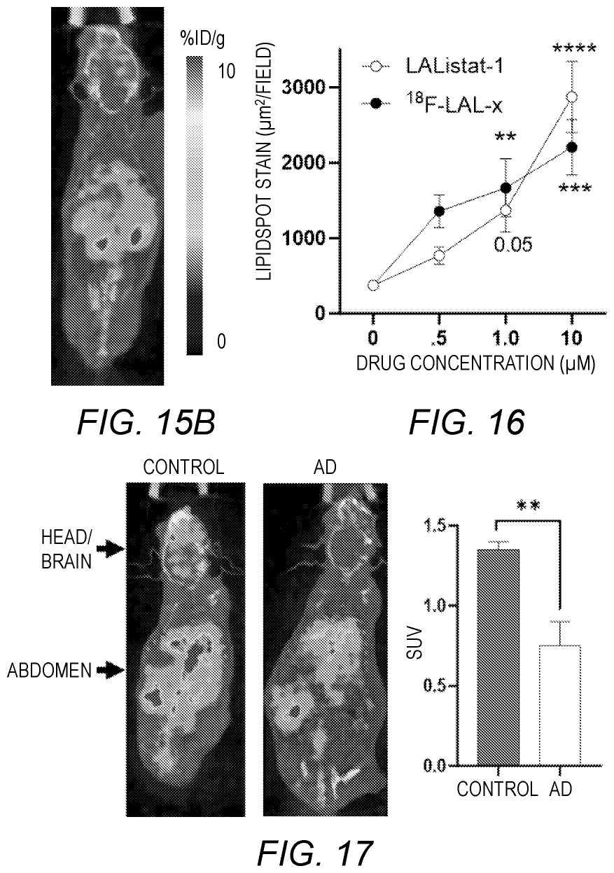

¹⁸F-LAL-x1 is a novel LAL PET ligand with rapid blood brain barrier (BBB) permeability. Given the ability of LAL loss to promote early Aβ pathology in rodents and perhaps humans, a family of new PET ligands for LAL was designed, coined ¹⁸F-LAL-x family. This includes ¹⁸F-LAL-x1, ¹⁸F-LAL-x2, and ¹⁸F-LAL-x3. The chemical synthesis schema for these exemplary compounds is in FIG. 14. For ¹⁸F-LAL-x1 an —[¹⁸F]CFH₂ group was introduced to the hetero-aliphatic ring of Lalistat 1 to minimize the impact of the label and maximize the BBB permeability possibility. The labeled motif was introduced to the C(3) oxygenated ring because this unit is transferred to the enzyme site after reaction, which can lead to better target retention. For LAL-x2 a longer side chain plus an amide bond is introduced to Lalistat 2. For LAL-x3 a photoredox radio-cyanation method was employed for direct modification. These PET ligands will allow for longitudinal monitoring of LAL levels in specific brain regions to identify individuals at risk for AD in the future. After the successful labeling, PET-CT was performed using ¹⁸F-LAL-x1 and found that it readily crosses the BBB in mice within 10 minutes with an uptake of 6.3% ID/g (FIGS. 15A-15B). Brain regions showing LAL labeling include AD impacted regions that Applicants found show LAL reductions in AD and with aging such as the cortex and hippocampus. Studies in brain slice culture found that the ¹⁸F-LAL-x1 retains its functional activity indicating it its specificity for LAL (FIG. 16).

Next, the ability of ¹⁸F-LAL-x1 was tested to measure differences in LAL between wild type control and AD mice (3×Tg-AD). ¹⁸F-LAL-x1 found a robust reduction in LAL in brain in the 10-month AD mouse compared to the wild type control (FIG. 17).

An additional synthetic scheme for LAL PET ligands is provided below:

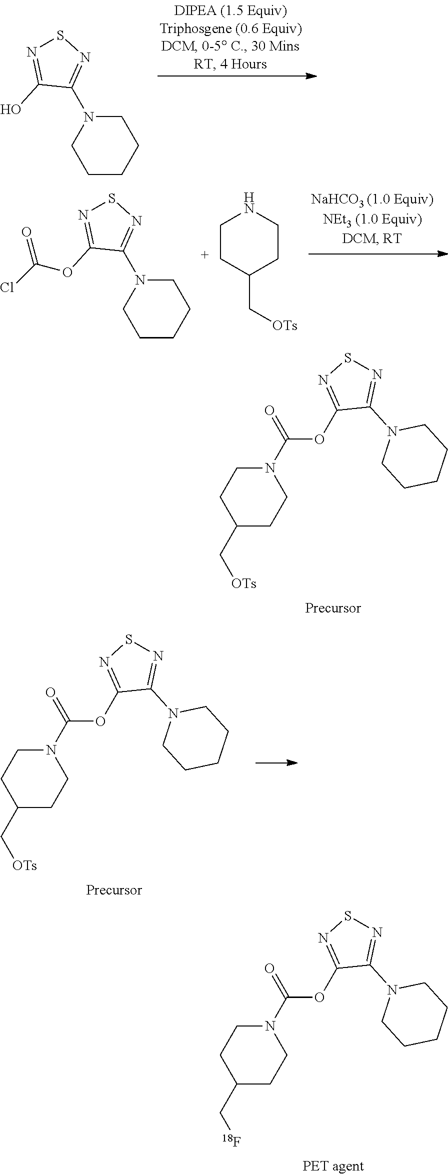

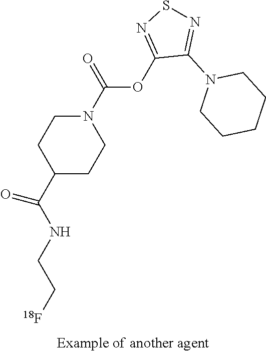 [SMILES: O=C(NCC[18F])C1CCN(C(=O)Oc2nsnc2N2CCCCC2)CC1]

TABLE 1
REAGENT or RESOURCE

| Antibodies | Isotype | Dilution | Clone | SOURCE; IDENTIFIER |
|---|---|---|---|---|
| Akt (pan) | Rabbit IgG | 1:1000 (WB) | Mono | Cell Signaling; 4685S |
| APP (6E10) | Mouse IgG | 1:500 (IF) | Mono | Biolegend; 803004 |
| APP [Y188] | Rabbit IgG | 1:1000 (WB) | Mono | Abcam; ab32136 |
| Aβ₁₋₄₂ | Rabbit IgG | 1:500 (IHC) | Mono | Invitrogen; 700254 |
| Aβ₁₋₄₂ | Rabbit IgG | 1:1000 (WB) | Mono | Abcam; ab201060 |
| BACE1 | Rabbit IgG | 1:1000 (WB) | Mono | Abcam; ab183612 |
| Beclin-1 | Rabbit IgG | 1:1000 (WB) | Mono | Cell Signaling; 3738S |
| CDK5 [EP715Y] | Rabbit IgG | 1:2000 (WB) | Mono | Abcam; ab40773 |
| FOXO1 | Rabbit IgG | 1:50 (CHIP) | Mono | Cell Signaling; 2880 |
| GAPDH | Mouse IgG | 1:2000 (WB) | Mono | Cell Signaling; 97166S |
| GAPDH | Chicken IgG | 1:5000 (WB) | Poly | Millipore; AB2302 |
| GAPDH | Mouse IgG | 1:2000 (WB) | Mono | Cell Signaling; 97166S) |
| GAPDH (14C10) | Rabbit IgG | 1:10,000 (WB) | Mono | Cell Signaling; 2118S |
| GSK3 alpha/bcta (p- | Rabbit IgG | 1:1000 (WB) | Poly | Invitrogen; 36646 |
| Tyr279) | | | | |
| GSK3β | Rabbit IgG | 1:1000 (WB) | Mono | Cell Signaling; 12456S |
| GSK3β (p-Ser9) | Rabbit IgG | 1:1000 (WB) | Mono | Cell Signaling; 5558S |
| Ibal | Rabbit IgG | 1:1000 (IHC) | Poly | Wako; 019-1974 |
| LAL | Rabbit IgG | 1:250 (IF), (WB) | Poly | Novus: NBP1-54155 |
| LAMP1 | Rabbit IgG | 1:100 (IF) | Mono | Abcam; ab208943 |
| LAMP1 | Rabbit IgG | 1:1000 (IHC, WB) | Mono | Abcam: ab208943 |
| LC3A/B | Rabbit IgG | 1:1000 (WB) | Mono | Cell Signaling; 12741S |
| MAP2 | Chicken IgG | 1:500 (IF) | Poly | Abcam; ab5392 |
| Normal Rabbit IgG | Rabbit IgG | 1:50 (CHIP) | — | Cell Signaling; 2729S |
| Phospho-Akt (Thr308) | Rabbit IgG | 1:1000 (WB) | Mono | Cell Signaling: 2965S |
| PKA C-α | Rabbit IgG | 1:1000 (WB) | Mono | Cell Signaling; 4782S |
| PP2a [E155] | Rabbit IgG | 1:5000 (WB) | Mono | Abcam: ab32104 |
| PSEN-1 [EP000Y] | Rabbit IgG | 1:1000 (WB) | Mono | Abcam; ab76083 |
| p-tau (Ser202, Thr205) | Rabbit IgG | 1:500 (WB) | Poly | Invitrogen; MN1020 |
| AT8 | | | | |
| p-tau (Ser214) | Rabbit IgG | 1:500 (WB) | Poly | Invitrogen; 44-742G. |
| phospho-Rpb1 (Pol II) | Rabbit IgG | 1:50 (CHIP) | Mono | Cell Signaling; 13499 |
| Tau [TAU-5] | Mouse IgG | 1:1000 (WB) | Mono | Abcam; ab80579 |
| Tau[pThr181] | Rabbit IgG | 1:200 (IHC), 1:500 | Poly | Novus; NB1008-2245 |
| | | (WB) | | |
| TRAIL | Rabbit IgG | 1:500 (IHC) | Poly | Abcam; ab42121 |

| Primers | Forward 5′→ 3′ | Reverse 5′ → 3′ |
|---|---|---|
| 18S | GGTAACCCGTTGAACCCCAT | CAACGCAAGCTTATGACCCG |
| | (SEQ ID NO: 1) | (SEQ ID NO: 2) |
| ABCA1 | AAAACCGCAGACATCCTTCAG | CATACCGAAACTCGTTCACCC |
| | (SEQ ID NO: 3) | (SEQ ID NO: 4) |
| ABCA2 | CCCGTCATGCAGTCGCTTT | CACTGGGTCGAACAAATTGCC |
| | (SEQ ID NO: 5) | (SEQ ID NO: 6) |
| ABCA5 | GATGTGGGAGTTTGGAGACAG | GCTGAATTTGTCCATAGGGCT |
| | (SEQ ID NO: 7) | (SEQ ID NO: 8) |
| ABCG1 | CTTTCCTACTCTGTACCCGAGG | CGGGGCATTCCATTGATAAGG |
| | (SEQ ID NO: 9) | (SEQ ID NO: 10) |
| Acox1 | TAACTTCCTCACTCGAAGCCA | AGTTCCATGACCCATCTCTGTC |
| | (SEQ ID NO: 11) | (SEQ ID NO: 12) |
| ATF4 | ATGGCGCTCTTCACGAAATC | ACTGGTCGAAGGGGTCATCAA |
| | (SEQ ID NO: 13) | (SEQ ID NO: 14) |
| ATP6v0c | ACTTATCGCTAACTCCCTGACT | ACACCAGCATCTCCGACGA |
| | (SEQ ID NO: 15) | (SEQ ID NO: 16) |
| ATP6v0b | AGTTGCTCTACCTCGGGATCT | ATGCCACATCAAAGCGAAAGC |
| | (SEQ ID NO: 17) | (SEQ ID NO: 18) |
| ATP6v0d1 | GCTACTTGGAGGGATTAGTGCG | GCGGAACTCTACTACCATCTTCT |
| | (SEQ ID NO: 19) | (SEQ ID NO: 20) |
| ATP6v1a | CTACCCAAAATCCGCGATGAG | CCATGTCACCTTCCAATCGAA |
| | (SEQ ID NO: 21) | (SEQ ID NO: 22) |
| ATP6v1b2 | ATGCGGGGAATCGTGAACG | AGGCTGGGATAGGTAGTTCCG |
| | (SEQ ID NO: 23) | (SEQ ID NO: 24) |
| ATP6v1d | GGCAAAGACCGGATTGAAATCT | GTCGAAATCGAAGAGTTAAGGCA |
| | (SEQ ID NO: 25) | (SEQ ID NO: 26) |
| ATP6ap2 | CTGGTGGCGGGTGCTTTAG | GCTACGTCTGGGATTCGATCT |
| | (SEQ ID NO: 27) | (SEQ ID NO: 28) |
| ATP6v1h | GGATGCTGCTGTCCCAACTAA | TCTCTTGCTTGTCCTCGGAAC |
| | (SEQ ID NO: 29) | (SEQ ID NO: 30) |
| APOE | CTCCCAAGTCACACAAGAACTG | CCAGCTCCTTTTTGTAAGCCTTT |
| | (SEQ ID NO: 31) | (SEQ ID NO: 32) |
| APP (human) | GTCCAGAATGGGAAGTGGGA | CACTGCATGTCTCTTTGGCG |
| | (SEQ ID NO: 33) | (SEQ ID NO: 34) |
| B2M | TTCTGGTGCTTGTCTCACTGA | CAGTATGTTCGGCTTCCCATTC |
| | (SEQ ID NO: 35) | (SEQ ID NO: 36) |
| C3 | CAGGACGTGAGAGTCGATGG | CTCTGCCTATGCTGCCTTCA |
| | (SEQ ID NO: 37) | (SEQ ID NO: 38) |
| CGI-58 | TGGTGTCCCACATCTACATCA | CAGCGTCCATATTCTGTTTCCA |
| | (SEQ ID NO: 39) | (SEQ ID NO: 40) |
| CHOP | CTGGAAGCCTGGTATGAGGAT | CAGGGTCAAGAGTAGTGAAGGT |
| | (SEQ ID NO: 41) | (SEQ ID NO: 42) |
| FOXO1 (human) | GGATGTGCATTCTATGGTGTACC | TTTCGGGATTGCTTATCTCAGAC |
| | (SEQ ID NO: 43) | (SEQ ID NO: 44) |
| Gpnmb | GCTGGTCTTCGGATGAAAATGA | CCACAAAGGTGATATTGGAACCC |
| | (SEQ ID NO: 45) | (SEQ ID NO: 46) |
| GRN | GGACACATGGCCTAGAATAACG | AGACACACCCTTAGAGAACGG |
| | (SEQ ID NO: 47) | (SEQ ID NO: 48) |
| GRP78 | ACTTGGGGACCACCTATTCCT | ATCGCCAATCAGACGCTCC |
| | (SEQ ID NO: 49) | (SEQ ID NO: 50) |
| HSL | GATTTACGCACGATGACACAGT | ACCTGCAAAGACATTAGACAGC |
| | (SEQ ID NO: 51) | (SEQ ID NO: 52) |
| IFNα | TACTCAGCAGACCTTGAACCT | CAGTCTTGGCAGCAAGTTGAC |
| | (SEQ ID NO: 53) | (SEQ ID NO: 54) |
| Iga1s | GGAGAGGGAATGATGTTGCCT | TCCTGCTTCGTGTTACACACA |
| | (SEQ ID NO: 55) | (SEQ ID NO: 56) |
| IL-1β | CTGGTGTGTGACGTTCCCATTA | CCGACAGCACGAGGCTTT |
| | (SEQ ID NO: 57) | (SEQ ID NO: 58) |
| IL-6 | GGCCTTCCCTACTTCACAAG | ATTTCCACGATTTCCCAGAG |
| | (SEQ ID NO: 59) | (SEQ ID NO: 60) |
| Lcad | TCTTTTCCTCGGAGCATGACA | GACCTCTCTACTCACTTCTCCAG |
| | (SEQ ID NO: 61) | (SEQ ID NO: 62) |
| LIPA (human) | TCTGGACCCTGCATTCTGAG | CACTAGGGAATCCCCAGTAAGAG |
| | (SEQ ID NO: 63) | (SEQ ID NO: 64) |
| LIPA (mouse) | TGTTCGTTTTCACCATTGGGA | CGCATGATTATCTCGGTCACA |
| | (SEQ ID NO: 65) | (SEQ ID NO: 66) |
| LPL | GGGAGTTTGGCTCCAGAGTTT | TGTGTCTTCAGGGGTCCTTAG |
| | (SEQ ID NO: 67) | (SEQ ID NO: 68) |
| LXRα | CTGATTCTGCAACGGAGTTGT | GACGAAGCTCTGTCGGCTC |
| | (SEQ ID NO: 69) | (SEQ ID NO: 70) |
| LXRβ | ATGTCTTCCCCCACAAGTTCT | GACCACGATGTAGGCAGAGC |
| | (SEQ ID NO: 71) | (SEQ ID NO: 72) |
| MAGL | AGGCGAACTCCACAGAATGTT | ACAAAAGAGGTACTGTCCGTCT |
| | (SEQ ID NO: 73) | (SEQ ID NO: 74) |
| MAPT (human) | CCAAGTGTGGCTCATTAGGCA | CCAATCTTCGGACTGGACTCTGT |
| | (SEQ ID NO: 75) | (SEQ ID NO: 76) |
| Mcad | AGGGTTTAGTTTTGAGTTGACGG | CCCCGCTTTTGTCATATTCCG |
| | (SEQ ID NO: 77) | (SEQ ID NO: 78) |
| MCP-1 | CCAGCCTACTCATTGGGA | GGGCCTGCTGTTCACAGTT |
| | (SEQ ID NO: 79) | (SEQ ID NO: 80) |
| PERK | GCGTCGGAGACAGTGTTTG | CGTCCATCTAAAGTGCTGATGAT |
| | (SEQ ID NO: 81) | (SEQ ID NO: 82) |
| PLIN | CTGTGTGCAATGCCTATGAGA | CTGGAGGGTATTGAAGAGCCG |
| | (SEQ ID NO: 83) | (SEQ ID NO: 84) |
| PLIN2 | GACCTTGTGTCCTCCGCTTAT | CAACCGCAATTTGTGGCTC |
| | (SEQ ID NO: 85) | (SEQ ID NO: 86) |
| PLIN3 | ATGTCTAGCAATGGTACAGATGC | CGTGGAACTGATAAGAGGCAGG |
| | (SEQ ID NO: 87) | (SEQ ID NO: 88) |
| PLIN4 | GTGTCCACCAACTCACAGATG | GGACCATTCCTTTTGCAGCAT |
| | (SEQ ID NO: 89) | (SEQ ID NO: 90) |
| PLIN5 | TGTCCAGTGCTTACAACTCGG | CAGGGCACAGGTAGTCACAC |
| | (SEQ ID NO: 91) | (SEQ ID NO: 92) |
| PNPLA2 | TCCGTGGCTGTCTACTAAAGA | TGGGATATGATGACGTTCTCTCC |
| | (SEQ ID NO: 93) | (SEQ ID NO: 94) |
| PPARα | AGAGCCCCATCTGTCCTCTC | ACTGGTAGTCTGCAAAACCAAA |
| | (SEQ ID NO: 95) | (SEQ ID NO: 96) |
| PPARγ | GGAAGACCACTCGCATTCCTT | GTAATCAGCAACCATTGGGTCA |
| | (SEQ ID NO: 97) | (SEQ ID NO: 98) |
| P2RY12 | CCCTGTGCGTCAGAGACTAC | CAAGCTGTTCGTGATGAGCC |
| | (SEQ ID NO: 99) | (SEQ ID NO: 100) |
| Snap29 | TTCGACGATGACGTGGAAGAG | GGTACTGCTGCCTGTCAATGG |
| | (SEQ ID NO: 101) | (SEQ ID NO: 102) |
| Srebp1 | TGACCCGGCTATTCCGTGA | CTGGGCTGAGCAATACAGTTC |
| | (SEQ ID NO: 103) | (SEQ ID NO: 104) |
| Srebp2 | GCAGCAACGGGACCATTCT | CCCCATGACTAAGTCCTTCAACT |
| | (SEQ ID NO: 105) | (SEQ ID NO: 106) |
| Stx17 | AAGTATCAGCGGTGCAGAATTT | CCTTGACAGGATCTATCATTCGC |
| | (SEQ ID NO: 107) | (SEQ ID NO: 108) |
| TGFβ | CTCCCGTGGCTTCTAGTGC | GCCTTAGTTTGGACAGGATCTG |
| | (SEQ ID NO: 109) | (SEQ ID NO: 110) |
| TLR4 | GCCTTTCAGGGAATTAAGCTCC | AGATCAACCGATGGACGTGTAA |
| | (SEQ ID NO: 111) | (SEQ ID NO: 112) |
| Tmem119 | TCTTCCGGCAGTACGTGATG | CGGCGCAGACTATGAACATGA |
| | (SEQ ID NO: 113) | (SEQ ID NO: 114) |
| TNFα | GACCCTCACACTCAGATCATCTTCT | CCTCCACTTGGTGGTTTGCT |
| | (SEQ ID NO: 115) | (SEQ ID NO: 116) |
| TRAIL | GGGAGTCCTCTCGGAAAGG | CCGGATAGCTGGTGTACTTGTA |
| | (SEQ ID NO: 117) | (SEQ ID NO: 118) |
| TREM2 | CTCCAGGAATCAAGAGACCTCC | CCGGGTCCAGTGAGGATCT |
| | (SEQ ID NO: 119) | (SEQ ID NO: 120) |
| TRPML1 | CTGACCCCCAATCCTGGGTAT | GGCCCGGAACTTGTCACAT |
| | (SEQ ID NO: 121) | (SEQ ID NO: 122) |
| VAMP8 | GGGAGTGCCGGAAATGACC | TGAAGTGTTCAGACGTGGCTT |
| | (SEQ ID NO: 123) | (SEQ ID NO: 124) |
| YKT6 | AGTCAACTGATTGTGGAACGC | TCTGGAAGGGTATTCGCTGTC |
| | (SEQ ID NO: 125) | (SEQ ID NO: 126) |

Primer sequences for ChIP analysis

| | | |
|---|---|---|
| LIPA Prepromoter | TCTGACAGAGACTGTTGCTCAACTT | AGCTGGGGCATTATTGGATGAA |
| (89255992-89256016) | (SEQ ID NO: 127) | (SEQ ID NO: 128) |
| LIPA Promoter | AAGCGCTTTAAATGGAGCCC | CTTCTGCGCCTGACAGAGAG |
| (89252180-89252199) | (SEQ ID NO: 129) | (SEQ ID NO: 130) |
| LIPA Exon 3 | TATTTGTGCCCGGTAGGCAG | TTCACGCTTGTGTTCCACCT |
| (89246089-89246108) | (SEQ ID NO: 131) | (SEQ ID NO: 132) |
| LIPA Exon 6 | AGTGTTAGGGCACACGGAAG | CCCACGCAAACAATACCACC |
| (89224681-89224700) | (SEQ ID NO: 133) | (SEQ ID NO: 134) |
| LIPA Exon 7 | TGAGGGTGACCTAACAACGC | CCTCCCTGAACAGAGGTTCG |
| (89224681-89224700) | (SEQ ID NO: 135) | (SEQ ID NO: 136) |
| LIPA Exon 8 | TCAATGCCACCTTAATGCTGTT | CCTGGAATGCCTACCTGGC |
| | (SEQ ID NO: 137) | (SEQ ID NO: 138) |
| LIPA Exon 10 | TCATGGCAGGCCATAGTTCT | GCGGGCTCCAAGAGTACATT |
| (89214060-89214079) | (SEQ ID NO: 139) | (SEQ ID NO: 140) |
| Human LIPA | AAGCGCTTTAAATGGAGCCC | CTTCTGCGCCTGACAGAGAG |
| | (SEQ ID NO: 141) | (SEQ ID NO: 142) |

Bacterial and virus strains

| | |
|---|---|
| AAV9.CD68.hM4di | VectorBuilder |
| PHP.eB-syn-scrambledRNA | VectorBuilder |
| PHP.eB-syn-shCON | VectorBuilder |
| (10¹² vg/mouse) | |
| PHP.eB-syn-WPRE-LAL | VectorBuilder |
| (10¹² vg/mouse) | |
| PHP.eB-syn-shLAL[1]-shLAL[2]- | VectorBuilder |
| shLAL[3] (10¹² vg/mouse) | |

Compounds

| | | |
|---|---|---|
| DA4-JC | MedChemExpress | HY-P3255 |
| Genistein | Sigma | G6649 |
| LAListat-2 | Tocris | 6099 |
| Lysotracker red | Thermo Fisher | L-7528 |
| Recombinant LAL/sebelipase | Creative Biomart | THP-0136 |
| Protein A Dynabeads | Thermo Fisher | 10008D |

Experimental models: Organisms/strains

| | | |
|---|---|---|
| Mouse: C57BL6/J WT | Jackson Laboratory | Stock No: 000664 |
| Mouse: 3xTg-AD | Jackson Laboratory | Stock No: 004807 |
| Mouse: CX3CR1.Cre^{ERT2} | Jackson Laboratory | Stock No: 021160 |
| Western Diet | Envigo | TD.88137 |

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A lysosomal acid lipase (LAL) positron emission tomography (PET) ligand according to: 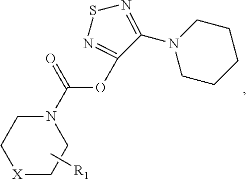
wherein X is C, CH, CH₂, CR₂, S, N, or O,
wherein R₁ can be substituted at one or more positions on the piperidyl ring and each R₁ is independently a C₁-C₅ alkyl, C₁-C₅ cyano, C₁-C₅ amide group, a linker (e.g., a PEG linker or an arene-containing linker), or a halogen, and comprises a radioisotope, and
wherein R₂ comprises a radiolabel.

2. The LAL PET ligand of claim 1, wherein the radioisotope is selected from fluorine-18, carbon-11, nitrogen-13 and oxygen-15.

3. The LAL PET ligand of claim 1, according to

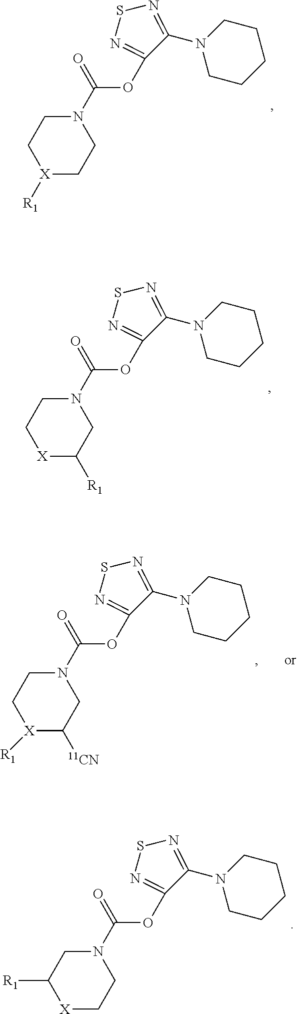

4. The LAL PET ligand of claim 1, selected from

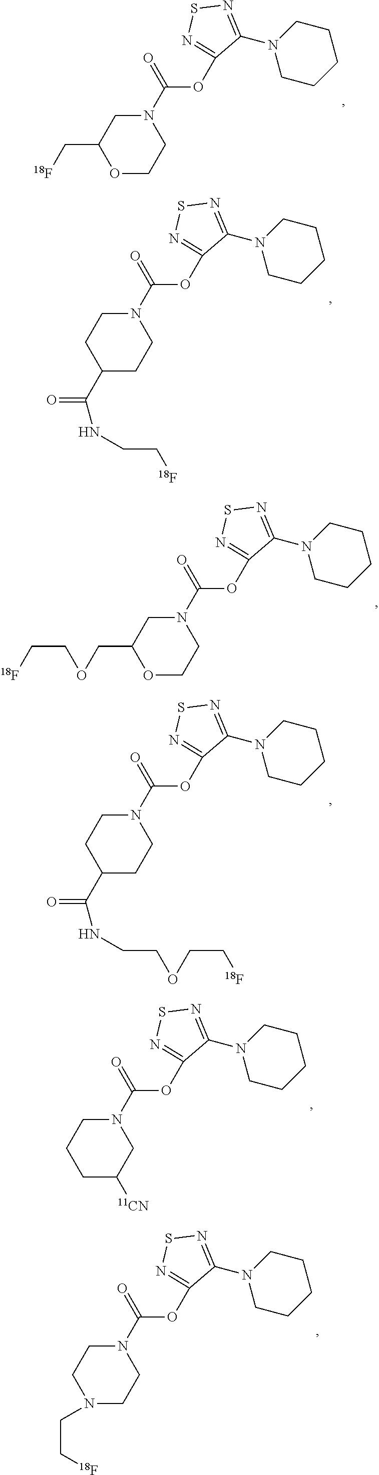

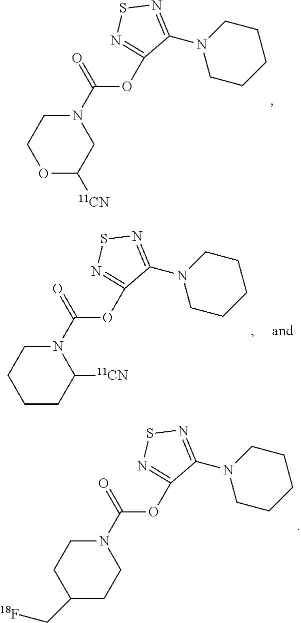

5. A composition comprising the ligand of claim 1.

6. A method of detecting the amount and/or activity of LAL in a target cell, the method comprising contacting the target cell with the LAL PET ligand of claim 1; and performing positron emission tomography on the cell.

7-9. (canceled)

10. A method of diagnosing or assessing the risk of developing Alzheimer's disease (AD) or Alzheimer's Disease Related Dementias (ADRD) in a subject, the method comprising:
a) measuring the amount and/or activity of lysosomal acid lipase (LAL) in the subject;
b) comparing the amount and/or activity of LAL measured in (a) with a threshold amount and/or activity of LAL; and
c) diagnosing AD or ADRD or identifying an increased risk of developing AD or ADRD based on the amount and/or activity of LAL measured in (a) being lower than the threshold amount and/or activity of LAL.

11. (canceled)

12. The method of claim 10, wherein the measuring the amount and/or activity of LAL in the subject comprises administering a LAL positron emission tomography (PET) ligand according to: 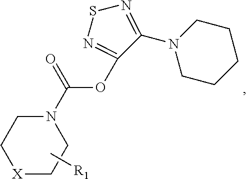
wherein X is C, CH, CH₂, CR₂, S, N, or O,
wherein R₁ can be substituted at one or more positions on the piperidyl ring and each R₁ is independently a C₁-C₅ alkyl, C₁-C₅ cyano, C₁-C₅ amide group, a linker (e.g., a PEG linker or an arene-containing linker, or a halogen, and comprises a radioisotope, and
wherein R₂ comprises a radiolabel and comprises a radioisotope; and
performing PET on the subject.

13-15. (canceled)

16. The method of claim 10, further comprising a step of administering a therapy to treat the subject based on the diagnosis or identification of the risk of developing AD/ADRD.

17-18. (canceled)

19. The method of claim 10, further comprising
(d) measuring the amount and/or activity of lysosomal lipid accumulation in the brain of the subject, and
(e) comparing the amount and/or activity of the lipid accumulation measured in (d) with a threshold amount of lipid accumulation, thereby diagnosing or identifying an increased risk of developing AD/ADRD based on the amount and/or activity of the lipid accumulation measured in (d) that is higher than the threshold amount and/or activity of the lipid accumulation.

20-21. (canceled)

22. The method of claim 19, wherein (d) is performed on cortex, hippocampus, parahippocampal regions, entorhinal regions, brainstem regions, or a combination thereof.

23-27. (canceled)

28. The method of claim 10, further comprising administering one or more standard AD/ADRD diagnostic tests.

29-32. (canceled)

33. The method of claim 16, wherein the therapy is selected from a group of enzyme replacement therapy, lipid-lowering drugs, dietary manipulation, or a combination thereof.

34. The method of claim 33, wherein the enzyme replacement therapy comprises administering LAL into the brain or central nervous system (CNS) of the subject.

35. The method of claim 33, wherein the enzyme replacement therapy comprises systemically administering LAL to the subject.

36-37. (canceled)

38. The method of claim 33, wherein the enzyme replacement therapy comprises administering a polynucleotide encoding LAL into the brain or CNS of the subject.

39. The method of claim 33, wherein the enzyme replacement therapy comprises systemically administering a polynucleotide encoding LAL to the subject.

40. The method of claim 38, wherein the polynucleotide is in a delivery vector.

41. The method of claim 40, wherein the delivery vector is a viral vector, optionally an adeno-associated virus (AAV) vector.

42. (canceled)

43. A method of treating a subject diagnosed with or at risk of developing AD/ADRD, the method comprising:
a) performing the method according to claim 10 to determine whether the subject has AD/ADRD or has an increased probability of developing AD/ADRD; and
b) administering a therapy to the subject based on the results of a).

44-53. (canceled)
